(19) United States
(12) Patent Application Publication
(10) Pub. No.: US 2007/0219212 A1
(43) Pub. Date: Sep. 20, 2007

(54) Pyranouidole Derivatives and the Use Thereof for the Treatment of Hepatitis C Virus Infection or Disease

(76) Inventors: Stephen M. Condon, Glenmoore, PA (US); Randy William Jackson, Glenmoore, PA (US); Matthew G. Laporte, Honey Brook, PA (US); Christopher J. Burns, Malvern, PA (US); Torsten Herbertz, Honey Brook, PA (US); Janet A. Gaboury, Blue Bell, PA (US)

Correspondence Address: Patrick J Hagan;Dann Dorfman Herrell and Skillman, 1601 Market Street, Suite 2400, Philadelphia, PA, 19103-2307, US

(21) Appl. No.: 10/589,453

(22) PCT Filed: Mar. 1, 2005
(86) PCT No.: PCT/US05/06645

§ 371 Date: May 1, 2007

Related U.S. Application Data
(60) Provisional application No. 60/549,019, filed on Mar. 1, 2004.

(51) Int. Cl. A61K 31/407; C07D 491/02

(52) U.S. Cl. 514/252.06; 514/411; 546/276.7; 548/364.4; 548/427; 549/396

(57) ABSTRACT

The invention is directed to novel pyranoindole derivatives and analogs as well as compositions containing the same and to the use thereof for the treatment, prevention or inhibition of viral infections and associated diseases caused by the Hepatitis C virus.

### BACKGROUND OF THE INVENTION

Hepatitis C is a common viral infection that can lead to chronic Hepatitis, cirrhosis, liver failure, and hepatocellular carcinoma. Infection with the Hepatitis C virus (HCV) leads to chronic Hepatitis in at least 85% of cases, is the leading reason for liver transplantation, and is responsible for at least 10,000 deaths annually in the United States (Hepatology, 1997, 26 (Suppl. 1), 2S-10S).

The Hepatitis C virus is a member of the Flaviviridae family, and the genome of HCV is a single-stranded linear RNA of positive sense (Hepatology, 1997, 26 (Suppl. 1), 11S-14S). HCV displays extensive genetic heterogeneity; at least 6 genotypes and more than 50 subtypes have been identified.

There is no effective vaccine to prevent HCV infection. The only therapy currently available is treatment with interferon-α (INF-α or combination therapy of INF-α with the nucleoside analog ribavirin (Antiviral Chemistry and Chemotherapy, 1997, 8, 281-301). However, only about 40% of treated patients develop a sustained response, so there is a need for more effective anti-HCV therapeutic agents.

The HCV genome contains a number of non-structural proteins: NS2, NS3, NS4A, NS4B, NS5A, and NS5B (J. General Virology, 2000, 81, 1631-1648). NS5B is an RNA-dependent RNA polymerase which is essential for viral replication, and therefore, the inhibition of NS5B is a suitable target for the development of therapeutic agents.

In the following U.S. patents, pyranoindole derivatives are disclosed and the compounds are stated to have antidepressant and antiulcer activity: U.S. Pat. No. 3,880,853 (Apr. 29, 1975), U.S. Pat. No. 4,118,394 (Oct. 3, 1978). In U.S. Pat. No. 4,179,503 (12/18/8) pyranoindoles are disclosed and stated to have diuretic activity. In the following U.S. patents, pyranoindole derivatives are disclosed and the compounds are stated to have antiinflammatory, analgesic, antibacterial, and antifungal activity: U.S. Pat. No. 3,843,681 (Oct. 22, 1974), U.S. Pat. No. 3,939,178 (Feb. 17, 1976), U.S. Pat. No. 3,974,179 (Aug. 10, 1976), U.S. Pat. No. 4,070,371 (Jan. 24, 1978), U.S. Pat. No. 4,076,831 (Feb. 28, 1978). In the following U.S. patents, pyranoindole derivatives are disclosed and the compounds are stated to have antiinflammatory and analgesic activity: U.S. Pat. No. 4,670,462 (Jun. 2, 1987), U.S. Pat. No. 4,686,213 (Aug. 11, 1987), U.S. Pat. No. 4,785,015 (Nov. 15, 1988), U.S. Pat. No. 4,810,699 (Mar. 7, 1989), U.S. Pat. No. 4,822,781 (Apr. 18, 1989), U.S. Pat. No. 4,960,902 (Oct. 2, 1990). In U.S. Pat. No. 5,776,967 (Jul. 7, 1998) and U.S. Pat. No. 5,830,911 (Nov. 3, 1998), pyranoindole derivatives are disclosed and the compounds are said to inhibit cyclooxegenase-2 and be useful for treating arthritic disorders, colorectal cancer, and Alzheimer's disease.

Also, in the following U.S. patents, processes for preparing pyranoindole derivatives are disclosed: U.S. Pat. No. 4,012,417 (Mar. 15, 1977), U.S. Pat. No. 4,036,842 (Jul. 19, 1977), U.S. Pat. No. 4,585,877 (Apr. 29, 1986), U.S. Pat. No. 4,822,893 (Apr. 18, 1989). Processes for the resolution of racemic pyranoindole derivatives are disclosed in the following U.S. patents: U.S. Pat. No. 4,501,899 (Feb. 26, 1985), U.S. Pat. No. 4,515,961 (May 7, 1985), U.S. Pat. No. 4,520,203 (May 28, 1985), U.S. Pat. No. 4,544,757 (Oct. 1, 1985).

Provisional Application No. 60/382,148 (filed on May 21, 2002) discloses methods of using pyranoindole compounds to treat infection with Hepatitis C. Provisional Application No. 60/382,154 (filed on May 21, 2002) discloses pyranoindole compositions useful for the treatment of Hepatitis C Infection or Disease.

### BRIEF SUMMARY OF THE INVENTION

This invention relates to pyranoindole derivatives, processes for their preparation and pharmaceutical compositions containing them, and to their use in the treatment of Hepatitis C viral infection.

In accordance with this invention there is provided a group of compounds represented by Formula I: 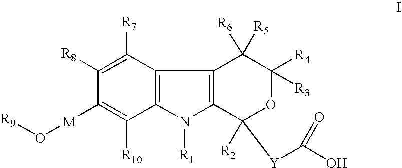
wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond, CH₂, or CH₂CH₂, with the proviso that when M is a bond, then R₉ is other than a hydroxyl, a straight chain alkyl of 1 to 8 carbon atoms, a branched alkyl of 3 to 12 carbons atoms, or an arylalkyl;
Y is a bond, CH₂, CH₂CH₂, aryl, or R₂ and Y together with the ring carbon atom to which they are attached may additionally form a spirocyclic cycloalkyl ring of 3 to 8 carbon atoms; or
a crystalline form or a pharmaceutically acceptable salt thereof.

Preferred compounds of the invention include the compounds of Formula I having the formula:

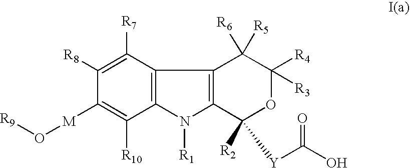

wherein R₁ through R₁₀ are as defined above.

A preferred embodiment of the invention include the compounds of Formula I wherein:

R₁-R₈, R₁₀, and Y are as defined above for compounds of Formula I;

R₉ is hydrogen, a straight chain alkyl of 1 to 8 carbon atoms, a branched alkyl of 3 to 12 carbon atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;

and M is CH₂ or CH₂CH₂; or a crystalline form or a pharmaceutically acceptable salt thereof.

Another preferred embodiment include the compounds of Formula I wherein:

R₁-R₈, R₁₀, and Y are as defined above for compounds of Formula I;

R₉ is a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, a substituted or unsubstituted heteroaryl of 7 to 12 carbon atoms, a substituted or unsubstituted heteroarylalkyl, a substituted or unsubstituted heterocyclic group, and a heterocycle-alkyl;

and M is a bond; or a crystalline form or a pharmaceutically acceptable salt thereof.

For purposes of this invention the term “alkyl” includes both straight and branched alkyl moieties, preferably of 1 to 8 carbon atoms. The term “alkenyl” refers to a radical aliphatic hydrocarbon containing at least one double bond and includes both straight and branched alkenyl-moieties of 2 to 7 carbon atoms. Such alkenyl moieties may exist in the E or Z configurations; the compounds of this invention include both configurations. The term “alkynyl” includes both straight chain and branched moieties containing 2 to 7 carbon atoms having at least one triple bond.

The term “cycloalkyl” refers to alicyclic hydrocarbon groups having 3 to 12 carbon atoms and includes but is not limited to: cyclopropyl, cyclobutyl, cyclopentyl, cyclohexyl, cycloheptyl, norbornyl, and adamantyl.

For purposes of this invention the term “aryl” is defined as an aromatic hydrocarbon moiety and may be substituted or unsubstituted. An aryl may be selected from but not limited to, the group: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. In one embodiment the substituted aryl may be optionally mono-, di-, tri- or tetra-substituted with substituents independently selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, amido, dialkylaminoalkyl, alkoxyiminoalkyl, hydroxyalkyl, cycloalkyl, alkylthio, —SO₃H, alkylsulfonyl, —SO₂NH₂, —SO₂NHalkyl, —SO₂N(alkyl)₂, alkylsulfonamido, alkenylsulfonamido, alkynylsulfonamido, —CO₂H, —CO₂NH₂, —CO₂NHalkyl, and —CO₂N(alkyl)₂. Preferred substituents for aryl include: alkyl, alkoxy, cycloalkyl, acyl, halogen, hydroxyalkyl, amino, alkylamino, dialkylamino, amido, alkylsulfonamido, alkylsulfonyl, —CO₂H, —CO₂NH₂, trifluoromethyl, trifluoromethoxy, arylalkyl, and alkylaryl.

For purposes of this invention the term “heteroaryl” is defined as an aromatic heterocyclic ring system (monocyclic or bicyclic), which may be substituted or unsubstituted, where the heteroaryl moieties are five or six membered rings containing 1 to 4 heteroatoms selected from the group consisting of S, N, and O, and include but is not limited to: (1) a monocyclic aromatic heterocycle such as furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-oxadiazole, 1,2,4-triazole, 1-methyl-1,2,4-triazole, 1,3,4-thiadiazole, 1,2,4-thiadiazole, 1H-tetrazole, and 1-methyltetrazole; (2) a bicyclic aromatic heterocycle where a phenyl, pyridine, pyrimidine or pyridizine ring is: (i) fused to a 6-membered aromatic (unsaturated) heterocyclic ring having one nitrogen atom; (ii) fused to a 5 or 6-membered aromatic (unsaturated) heterocyclic ring having two nitrogen atoms; (iii) fused to a 5-membered aromatic (unsaturated) heterocyclic ring having one nitrogen atom together with either one oxygen or one sulfur atom; or (iv) fused to a 5-membered aromatic (unsaturated) heterocyclic ring having one heteroatom selected from O, N or S. Bicyclic aromatic heterocycles include, but are not limited to, benzoxazole, benzothiazole, benzofuran, benzisoxazole, benzimidazole, N-methylbenzimidazole, azabenzimidazole, indazole, quinazoline, quinoline, and pyrrolidine. In one embodiment the substituted heteroaryl may be optionally mono-, di-, tri- or tetra-substituted with substituents independently selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, amido, dialkylaminoalkyl, alkoxyiminoalkyl, hydroxyalkyl, cycloalkyl, alkylthio, —SO₃H, alkylsulfonyl, —SO₂NH₂, —SO₂NHalkyl, —SO₂N(alkyl)₂, alkylsulfonamido, alkenylsulfonamido, alkynylsulfonamido, —CO₂H, —CO₂NH₂, —CO₂NHalkyl, and —CO₂N(alkyl)₂. Preferred substituents for heteroaryl include: alkyl, alkoxy, cycloalkyl, acyl, halogen, hydroxyalkyl, amino, alkylamino, dialkylamino, amido, alkylsulfonamido, alkylsulfonyl, —CO₂H, —CO₂NH₂, trifluoromethyl, trifluoromethoxy, arylalkyl, and alkylaryl.

For the purposes of this invention the term “alkoxy” is defined as C1-C12-alkyl-O—; the term “aryloxy” is defined as aryl-O—; the term “heteroaryloxy” is defined as heteroaryl-O—; wherein alkyl, aryl, and heteroaryl are as defined above.

For purposes of this invention the term “arylalkyl” is defined as aryl-C1-C6-alkyl-, and may be substituted or unsubstituted; arylalkyl moieties include benzyl, 1-phenylethyl, 2-phenylethyl, 3-phenylpropyl, 2-phenylpropyl and the like. In one embodiment the substituted arylalkyl may be optionally mono-, di-, tri- or tetra-substituted with substituents independently selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, amido, dialkylaminoalkyl, alkoxyiminoalkyl, hydroxyalkyl, cycloalkyl, alkylthio, —SO₃H, alkylsulfonyl, —SO₂NH₂, —SO₂NHalkyl, —SO₂N(alkyl)₂, alkylsulfonamido, —CO₂H, —CO₂NH₂, —CO₂NHalkyl, and —CO₂N(alkyl)₂. Preferred substituents for arylalkyl include: alkyl, alkoxy, cycloalkyl, acyl, halogen, hydroxyalkyl, amino, alkylamino, dialkylamino, amido, alkylsulfonamido, alkylsulfonyl, —CO₂H, —CO₂NH₂, trifluoromethyl, trifluoromethoxy, arylalkyl, and alkylaryl.

For purposes of this invention the term “heteroarylalkyl” is defined as heteroaryl-C1-C6-alkyl-, and may be substituted or unsubstituted. In one embodiment the substituted heteroarylalkyl may be optionally mono-, di-, tri- or tetra-substituted with substituents independently selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, amido, dialkylaminoalkyl, alkoxyiminoalkyl, hydroxyalkyl, cycloalkyl, alkylthio, —SO₃H, alkylsulfonyl, —SO₂NH₂, —SO₂NHalkyl, —SO₂N(alkyl)₂, alkylsulfonamido, morpholine, —CO₂H, —CO₂NH₂, —CO₂NHalkyl, and —CO₂N(alkyl)₂. Preferred substituents for heteroarylalkyl include: alkyl, alkoxy, cycloalkyl, acyl, halogen, hydroxyalkyl, amino, alkylamino, dialkylamino, amido, alkylsulfonamido, alkylsulfonyl, —CO₂H, —CO₂NH₂, morpholine, trifluoromethyl, trifluoromethoxy, arylalkyl, and alkylaryl.

For purposes of this invention the term “alkylaryl” is defined as C1-C6-alkyl-aryl-.

For purposes of this invention the term “alkylthio” is defined as C1-C6-alkyl-S—.

For purposes of this invention “alkoxyalkyl,” “cycloalkyl-alkyl,” “alkylthioalkyl,” “aryloxyalkyl,” and “heteroaryloxyalkyl” denote an alkyl group as defined above that is further substituted with an alkoxy, cycloalkyl, alkylthio, aryloxy, or heteroaryloxy group as defined above.

For purposes of this invention “arylalkoxy,” “alkoxyalkoxy” and “cycloalkylalkoxy” denote an alkoxy group as defined above that is further substituted with an aryl, alkoxy, alkylthio, cycloalkyl or heteroaryl group as defined above.

For purposes of this invention “arylthioalkyl” and “heteroarylthioalkyl” denote an alkyl group as defined above that is further substituted with an arylthio or heteroarylthio group as defined above.

For purposes of this invention “aryloxyalkylthio” is defined as aryloxy-C1-C8-alkyl-S—; “heteroaryloxyalkylthio” is defined as heteroaryloxy-C1-C8-alkyl-S—; where aryloxy, heteroaryloxy, and alkyl are defined above.

For purposes of this invention “alkoxyimino” is defined as alkoxy-N═C—; alkoxyiminoalkyl denotes an alkyl group as defined above that is further substituted with an alkoxyimino group.

For purposes of this invention “arylalkoxyalkyl,” “alkoxyalkoxyalkyl,” and “cycloalkylalkoxyalkyl” denotes an alkyl group as defined above that is further substituted with an arylalkoxy, alkoxyalkoxy, or cycloalkylalkoxy as defined above.

For purposes of this invention “phenylalkynyl” is an alkynyl group further substituted with a phenyl group.

In the most preferred embodiment of this invention a substituted methyl comprises a methyl substituent further substituted with, for example, a furanyl group. In another embodiment of this invention a furanyl substituent is further substituted with a methyl group.

In a preferred embodiment of this invention trifluoromethoxy is CF₃O—. In another embodiment of this invention trifluoromethylthio is CF₃S—.

In one embodiment of this invention trifluoroethoxy includes but is not limited to CF₃CH₂O—. In another embodiment of this invention trifluoroethylthio includes but is not limited to CF₃CH₂S—.

The terms “monoalkylamino” and “dialkylamino” refer to moieties with one or two alkyl groups wherein the alkyl chain is 1 to 8 carbons and the groups may be the same or different. The terms monoalkylaminoalkyl and dialkylaminoalkyl refer to monoalkylamino and dialkylamino moieties with one or two alkyl groups (the same or different) bonded to the nitrogen atom which is attached to an alkyl group of 1 to 8 carbon atoms. The terms “substituted monoalkylaminoalkyl” and “substituted dialkylaminoalkyl” refer to monoalkylaminoalkyl and dialkylaminoalkyl moieties that are further substituted with one or more substituents independently selected from the group consisting of aryl.

“Acyl” is a radical of the formula —(C═O)-alkyl or —(C═O)-perfluoroalkyl wherein the alkyl radical or perfluoroalkyl radical is 1 to 7 carbon atoms; preferred examples include but are not limited to, acetyl, propionyl, butyryl, trifluoroacetyl.

“Amido” is a radical of the formula —NH(C═O)-alkyl or —N(alkyl)(C═O)-alkyl, wherein the alkyl(s) radical are independently 1 to 7 carbon atoms.

For purposes of this invention the term “alkylsulfinyl” is defined as a R′SO— radical, where R′ is an alkyl radical of 1 to 8 carbon atoms. Alkylsulfonyl is a R′SO₂— radical, where R′ is an alkyl radical of 1 to 8 carbon atoms. An alkylsulfonylalkyl is an alkyl group of 1 to 8 carbons that is further substituted with an alkylsulfonyl group. Alkylsulfonamido, alkenylsulfonamido, alkynylsulfonamido are R′SO₂NR″— radicals, where R′ is an alkyl radical of 1 to 8 carbon atoms, an alkenyl radical of 2 to 8 carbon atoms, or an alkynyl radical of 2 to 8 carbon atoms, respectively and R″ is hydrogen or an alkyl radical of 1 to 8 carbon atoms.

For purposes of this invention the term “heterocyclic group” is defined as a heterocyclic ring system (monocyclic or bicyclic) containing at least one saturated or partially saturated heteroaryl group as is defined above, and which may be substituted or unsubstituted. Heterocyclic groups include, but are not limited to (1) a monocyclic saturated or partially saturated heteroaryl group of five or six members containing 1 to 4 heteroatoms selected from the group consisting of S, N, and O; and (2) a bicyclic heterocycle where a phenyl, pyridine, pyrimidine or pyridizine ring is fused to a 5 or 6-membered saturated or partially saturated heteroaryl group containing 1 to 4 heteroatoms selected from the group consisting of S, N, and O. Examples of heterocyclic groups include, but are not limited to, the moieties: azetidinyl, 1,4-dioxane, 1,3-dioxolane, hexahydroazepine, piperazine, piperidine, pyrrolidine, morpholine, thiomorpholine, dihydrobenzimidazole, dihydrobenzofuran, dihydrobenzothiene, dihydrobenzoxazole, dihydrofuran, dihydroimidazole, dihydroindole, dihydroisooxazole, dihydroisothiazole, dihydrooxadiazole, dihydrooxazole, dihydropyrrazine, dihydropyrazole, dihydropyridine, dihydropyrimidine, dihydropyrrole, dihydroquinoline, dihydrotetrazole, dihydrothiadiazole, dihydrothiazole, dihydrothien, dihydrotriazole, dihydroazetidine, dihydro-1,4-dioxane, tetrahydrofuran, tetrahydropyran, tetrahydrothien, tetrahydrothiopyran, tetrahydroquinoline, and tetrahydroisoquinoline. In one embodiment the substituted heteroaryl may be optionally mono-, di-, tri- or tetra-substituted with substituents independently selected from, but not limited to, the group consisting of carbonyl and alkylsulfonyl.

For purposes of this invention, the term “heterocycle-alkyl” denotes an alkyl group as defined above that is further substituted with a heterocyclic group as defined above.

For purposes of this invention, the term “BB7” denotes an RNA-dependent RNA polymerase hepatitis C virus protein sequence which is derived from HCV replicon. A discussion of BB7 and related technology can be found in Blight, K. et al. (2000) Science 290:1972-1974. BB7 can be licensed from Apath, LLC (893 North Warson Road, Saint Louis Mo. 63141, USA). BB7 is also referred to as Con1 HCV sequence and discussions of Con1 can be found in the following references: Lohmann, V. et al. (1999) Science 285:110-113; Pietschmann, T. et al. (2001) J. Virol. 73:1252-1264; Lohmann, V. et al. (2001) J. Virol. 75:1437-1449.

The compounds of this invention may contain an asymmetric carbon atom and some of the compounds of this invention may contain one or more asymmetric centers and may thus give rise to stereoisomers, such as enantiomers and diastereomers. The stereoisomers of the instant invention are named according to the Cahn-Ingold-Prelog System. While shown without respect to stereochemistry in Formula I, the present invention includes all the individual possible stereoisomers; as well as the racemic mixtures and other mixtures of R and S stereoisomers (scalemic mixtures which are mixtures of unequal amounts of enantiomers) and pharmaceutically acceptable salts thereof. It should be noted that stereoisomers of the invention having the same relative configuration at a chiral center may nevertheless have different R and S designations depending on the substitution at the indicated chiral center.

For compounds of this invention containing two chiral centers, four possible stereoisomers are possible; these four stereoisomers are classified as two racemic pairs of diastereomers. These compounds of the invention may be present as racemic diastereomers which would be designated following the convention described in the 1997 Chemical Abstracts Index Guide, Appendix IV (Columbus, Ohio) whereas the first cited chiral atom is designated R* and the next cited chiral atom is designated R* if it possesses the same chirality as the first cited stereocenter or S* if it possesses opposite chirality to the first cited stereocenter. Alternatively, these compounds of the invention may be present as non-racemic mixtures of two diastereomers owing to the existence of a predefined stereocenter. In these instances, the predefined stereocenter is assigned based on the Cahn-Ingold-Prelog System and the undefined stereocenter is designated R* to denote a mixture of both R and S stereoisomers at this center. Compounds of this invention which possess two chiral centers but which are present as single stereoisomers are described using the Cahn-Ingold-Prelog System.

Based on the chiral center at the C1 carbon position in Formula I, a preferred embodiment of the instant invention is the compound of Formula I(a) shown below: 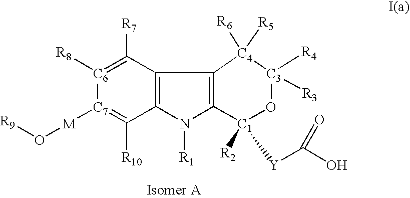
The configuration at C1 in Formula I(a) for purposes of this invention is also referred to as “Isomer A”, and the opposite configuration at C1 is herein defined as “Isomer B” and has the Formula I(b) shown below:

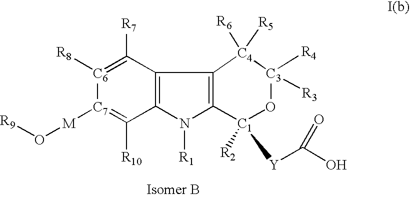

In one embodiment of this invention the compound of the invention is comprised of a ratio of Isomer A to Isomer B of greater than 1:1. In the most preferred embodiment the compound is comprised of 100% Isomer A. In further embodiments the compound is comprised of a ratio of Isomer A to Isomer B of at least about 9:1. In another embodiment the compound is comprised of a ratio of Isomer A to Isomer B of at least about 8:1. Additionally the compound is comprised of a ratio of Isomer A to Isomer B of at least about 7:1.

Another embodiment of this invention is where R₂ of Formula I is a sec-butyl group. In a preferred embodiment, the chiral carbon of the sec-butyl group has an S to R configuration ratio of 1:1. In further embodiments, the chiral carbon of the sec-butyl group has an S to R configuration ratio selected from the group consisting of at least 7:1, at least 8:1, and at least 9:1. In a most preferred embodiment of the invention, the chiral carbon of the sec-butyl group has 100% S configuration.

A preferred aspect of this invention, includes the compounds of Formula I(c):

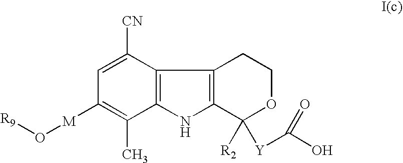

wherein R₂, R₉, M, and Y are as defined for compounds of Formula 1, above.

In further embodiments of the compound of this invention, the compound may be selected from any of the compounds described, supra.

The present invention provides a pharmaceutical composition comprising a compound of Formula I:

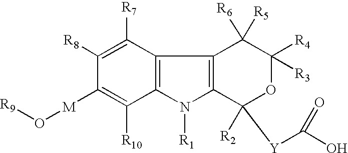

wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl, a cycloalkylalkoxyalkyl, hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond, CH₂, or CH₂CH₂, with the proviso that when M is a bond, then R₉ is other than a hydroxyl, a straight chain alkyl of 1 to 8 carbon atoms, a branched alkyl of 3 to 12 carbons atoms, or an arylalkyl;
Y is a bond, CH₂, CH₂CH₂, aryl, or R₂ and Y together with the ring carbon atom to which they are attached may additionally form a spirocyclic cycloalkyl ring of 3 to 8 carbon atoms; or a crystalline form or a pharmaceutically acceptable salt thereof; and a pharmaceutically acceptable carrier medium.

The present invention also provides a pharmaceutical composition comprising a compound of Formula I(c):

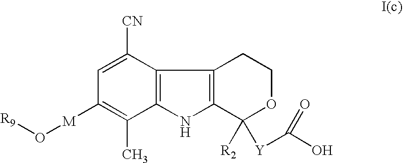

wherein R₂, R₉, M, and Y are as defined for compounds of Formula 1, and a pharmaceutically acceptable carrier medium.

Pharmaceutically acceptable salts of the compounds of Formula I having acidic moieties at R₃, R₄, R₅, R₆, R₇, R₈, R₉, or R₁₀ may be formed from organic and inorganic bases. For example alkali metal salts: sodium, lithium, or potassium and N-tetraalkylammonium salts such as N-tetrabutylammonium salts. Similarly, when a compound of this invention contains a basic moiety at R₃, R₄, R₅, R₆, R₇, R₈, R₉, or R₁₀, salts can be formed from organic and inorganic acids. For example salts can be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids.

In one embodiment, the present invention provides for a method of inhibiting the Hepatitis C RNA-dependent RNA polymerase NS5B. The method comprises contacting a cell with an amount of a compound effective to decrease or prevent NS5B function. The cell may be a mammalian cell and more specifically a human cell. The cell may also be a bacterial cell such as for example E coli. The cell may include but is not limited to, a neuronal cell, an endothelial cell, a glial cell, a microglial cell, a smooth muscle cell, a somatic cell, a bone marrow cell, a liver cell, an intestinal cell, a germ cell, a myocyte, a mononuclear phagocyte, an endothelial cell, a tumor cell, a lymphocyte cell, a mesangial cell, a retinal epithelial cell, a retinal vascular cell, a ganglion cell or a stem cell. The cell may be a normal cell, an activated cell, a neoplastic cell, a diseased cell, or an infected cell.

In another embodiment, the present invention provides a method for the treatment or prevention of Hepatitis C infection in a mammal. The present invention accordingly provides to a mammal, a pharmaceutical composition that comprises a compound of this invention in combination or association with a pharmaceutically acceptable carrier. The compound of this invention may be administered as the sole therapeutic agent or in combination with other therapeutically effective compounds or therapies for the treatment or prevention of Hepatitis C viral infection in a mammal.

The compounds are preferably provided orally or subcutaneously. The compounds may be provided by intralesional, intraperitoneal, intramuscular or intravenous injection; infusion; liposome-mediated delivery; topical, nasal, anal, vaginal, sublingual, uretheral, transdermal, intrathecal, ocular or otic delivery. In order to obtain consistency in providing the compound of this invention it is preferred that a compound of the invention is in the form of a unit dose. Suitable unit dose forms include tablets, capsules and powders in sachets or vials. Such unit dose forms may contain from 0.1 to 100 mg of a compound of the invention and preferably from 2 to 50 mg. Still further preferred unit dosage forms contain 5 to 25 mg of a compound of the present invention. The compounds of the present invention can be administered orally at a dose range of about 0.01 to 100 mg/kg or preferably at a dose range of 0.1 to 10 mg/kg. Such compounds may be administered from 1 to 6 times a day, more usually from 1 to 4 times a day. The effective amount will be known to one of skill in the art; it will also be dependent upon the form of the compound. One of skill in the art could routinely perform empirical activity tests to determine the bioactivity of the compound in bioassays and thus determine the appropriate dosage to administer.

The compounds of the invention may be formulated with conventional excipients, such as a filler, a disintegrating agent, a binder, a lubricant, a flavoring agent, a color additive, or a carrier. The carrier may be for example a diluent, an aerosol, a topical carrier, an aqueous solution, a nonaqueous solution or a solid carrier. The carrier may be a polymer or a toothpaste. A pharmaceutically acceptable carrier medium in this invention encompasses any of the standard pharmaceutically accepted carriers, such as phosphate buffered saline solution, acetate buffered saline solution, water, emulsions such as an oil/water emulsion or a triglyceride emulsion, various types of wetting agents, tablets, coated tablets and capsules.

When provided orally or topically, such compounds would be provided to a subject by delivery in different carriers. Typically, such carriers contain excipients such as starch, milk, sugar, certain types of clay, gelatin, stearic acid, talc, vegetable fats or oils, gums, or glycols. The specific carrier would need to be selected based upon the desired method of delivery, for example, phosphate buffered saline (PBS) could be used for intravenous or systemic delivery and vegetable fats, creams, salves, ointments or gels may be used for topical delivery.

The compounds of the present invention may be delivered together with suitable diluents, preservatives, solubilizers, emulsifiers, adjuvants and/or carriers useful in treatment or prevention of Hepatitis C viral infection. Such compositions are liquids or lyophilized or otherwise dried formulations and include diluents of various buffer content (for example, Tris-HCl, acetate, phosphate), pH and ionic strength, additives such as albumins or gelatin to prevent absorption to surfaces, detergents (for example, TWEEN 20, TWEEN 80, PLURONIC F68, bile acid salts), solubilizing agents (for example, glycerol, polyethylene glycerol), anti-oxidants (for example ascorbic acid, sodium metabisulfate), preservatives (for example, thimerosal, benzyl alcohol, parabens), bulking substances or tonicity modifiers (for example, lactose, mannitol), covalent attachment of polymers such as polyethylene glycol, complexation with metal ions, or incorporation of the compound into or onto particulate preparations of hydrogels or liposomes, micro-emulsions, micelles, unilamellar or multilamellar vesicles, erythrocyte ghosts, or spheroplasts. Such compositions will influence the physical state, solubility, stability, rate of in vivo release, and rate of in vivo clearance of the compound or composition. The choice of compositions will depend on the physical and chemical properties of the compound capable of treating or preventing a Hepatitis C viral infection.

The compounds of the present invention may be delivered locally via a capsule that allows a sustained release of the compound over a period of time. Controlled or sustained release compositions include formulation in lipophilic depots (for example, fatty acids, waxes, oils).

The present invention further provides a compound of the invention for use as an active therapeutic substance for preventing Hepatitis C infection. Compounds of Formula I are of particular use for the treatment of infection with Hepatitis C virus.

The present invention further provides a method of treating Hepatitis C infection in humans, which comprises administering to the infected individual an effective amount of a compound or a pharmaceutical composition of the invention.

The present invention further provides controlled-release therapeutic dosage forms for the pharmaceutical composition in which the composition is incorporated into a delivery system. The dosage form controls release of the pharmaceutical composition in such a manner that an effective concentration of the composition in the blood can be maintained over an extended period of time, but also the release of the composition should be such that the concentration in the blood remains relatively constant over the extended period of time to improve therapeutic results and/or minimize side effects. Additionally, a controlled release system would affect minimal peak to trough fluctuations in blood plasma levels of the pharmaceutical composition.

The present invention provides a method of treating or preventing a Hepatitis C viral infection in a mammal comprising providing the mammal with an effective amount of at least one pharmaceutical composition, wherein the at least one pharmaceutical composition includes a compound of a formula: 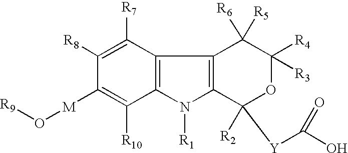
wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond, CH₂, or CH₂CH₂, with the proviso that when M is a bond, then R₉ is other than a hydroxyl, a straight chain alkyl of 1 to 8 carbon atoms, a branched alkyl of 3 to 12 carbons atoms, or an arylalkyl;
Y is a bond, CH₂, CH₂CH₂, aryl, or R₂ and Y together with the ring carbon atom to which they are attached may additionally form a spirocyclic cycloalkyl ring of 3 to 8 carbon atoms; or
a crystalline form or a pharmaceutically acceptable salt thereof.

The present invention also provides a method of treating or preventing a Hepatitis C viral infection in a mammal comprising providing the mammal with an effective amount of at least one pharmaceutical composition, wherein the at least one pharmaceutical composition includes a compound of Formula 1(c):

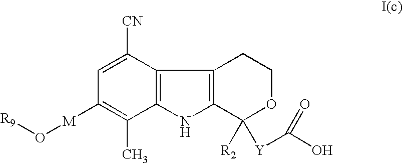

wherein R₂, R₉, M, and Y are as defined for compounds of Formula 1, above.

The method of the present invention further comprises providing the mammal with an effective amount of at least one biologically active agent.

In an embodiment of the method of the present invention, the at least one biologically active agent is provided prior to the at least one pharmaceutical composition, concurrently with the at least one pharmaceutical composition or after the at least one pharmaceutical composition. In a further embodiment of the method of the present invention, the compound is a crystalline form or a pharmaceutically acceptable salt thereof.

In a further embodiment of the method of the present invention, the at least one biologically active agent is selected from the group consisting of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds. In a further embodiment the at least one biologically active agent is a pegylated interferon. In a yet further embodiment the pegylated interferon is a pegylated interferon-alpha.

The compounds of the present invention or precursors thereof and their isomers and pharmaceutically acceptable salts thereof are also useful in treating and preventing viral infections, in particular hepatitis C infection, and diseases in living hosts when used in combination with each other (i.e. pharmaceutical compositions comprising the compounds are administered concurrently with each or sequentially, in either order). The combination of compounds provided herein may further be provided to a subject in respective pharmaceutical compositions, concurrently with or sequentially to other biologically active agents, including but not limited to the group consisting of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds. The present invention further provides combination therapy with one or more pyranoindole derivatives, i.e., at least two pharmaceutical compositions, each comprising a different compound of the present invention, are provided to a subject in need thereof either concurrently with each other or sequentially, and such therapy may further comprise providing concurrently or sequentially other medicinal agents or potentiators, such as acyclovir, famicyclovir, valgancyclovir and related compounds, ribavirin and related compounds, amantadine and related compounds, various interferons such as, for example, interferon-alpha, interferon-beta, interferon-gamma and the like, as well as alternative forms of interferons such as pegylated interferons. Additionally, combinations of, for example ribavirin and interferon, may be administered as an additional combination for a multiple combination therapy with at least one of the compounds of the present invention.

The combination therapy with any of the above-described biologically active agents may also be sequential, that is the treatment with a first pharmaceutical composition comprising a compound of the invention followed by treatment with a second pharmaceutical composition comprising a second compound of the invention, wherein the second compound is different than the first compound; alternatively, treatment may be with both two or more pharmaceutical compositions, wherein each pharmaceutical composition comprises a different compound of the invention, at the same time. The sequential therapy can be within a reasonable time after the completion of the first therapy with the pharmaceutical composition. Treatment with the respective pharmaceutical compositions, each comprising a different compound of the present invention, at the same time may be provided in the same daily dose or in separate doses. Combination therapy may also be provided wherein a pharmaceutical composition comprising at least one compound of the present invention is administered in a composition further comprising at least one biologically active agent, i.e. in a single dose. The dosages for both concurrent and sequential combination therapy (for combined pharmaceutical compositions comprising at least two compounds of the invention or compositions comprising at least one compound of the invention and at least one biologically active agent), will depend on absorption, distribution, metabolism and excretion rates of the components of the pharmaceutical composition as well as other factors known to one of skill in the art. Dosage values of the pharmaceutical composition will also vary with the severity of the condition to be alleviated. It is to be further understood that for any particular subject, specific dosage regimens and schedules may be adjusted over time according to the individual's need and the professional judgment of the person administering or supervising the administration of the pharmaceutical compositions.

In a further embodiment, the compounds of the invention may be used for the treatment of HCV in humans in combination therapy mode with other inhibitors of the HCV polymerase.

In yet a further embodiment, the compounds of the present invention may be used for the treatment of HCV in humans in combination therapy mode with other inhibitors of the HCV life cycle such as, for example, inhibitors of HCV cell attachment or virus entry, HCV translation, HCV RNA transcription or replication, HCV maturation, assembly or virus release, or inhibitors of HCV enzyme activities such as the HCV nucleotidyl transferase, helicase, protease or polymerase.

It is intended that combination therapies of the pharmaceutical compositions include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

The term “interferon-alpha” as used herein means the family of highly homologous species-specific proteins that inhibit viral replication and cellular proliferation and modulate immune response. Typical suitable interferon-alphas include, but are not limited to, recombinant interferon alpha-2b such as INTRON-A INTERFERON available from Schering Corporation, Kenilworth, N.J., recombinant interferon alpha-2a such as Roferon interferon available from Hofman-La Roche, Nutley, N.J., a recombinant interferon alpha-2C, such as BEROFOR ALPHA 2 INTERFERON available from Boehringer Ingelheim Pharmaceutical, Inc., Ridgefield, Conn., interferon alpha-n1, a purified blend of natural alpha interferons such as SUMIFERON available from Sumitomo, Japan or as Wellferon interferon alpha-n1 (INS) available from Glaxo-Wellcome Ltd., London, Great Britain, or a consensus alpha interferon such as those described in U.S. Pat. Nos. 4,897,471 and 4,695,623 (the contents of which are hereby incorporated by reference in their entireties, specifically examples 7, 8 or 9 thereof) and the specific product available from Amgen, Inc., Newbury Park, Calif., or interferon alpha-n3a mixture of natural interferons made by Interferon Sciences and available from the Purdue Frederick Co., Norwalk, Conn., under the ALFERON trademark. The use of interferon alpha-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,503,901.

The term “pegylated interferon” as used herein means polyethylene glycol modified conjugates of interferon, preferably interferon alpha-2a and alpha-2b. The preferred polyethylene-glycol-interferon alpha-2b conjugate is PEG.sub.12000-interferon alpha 2b. The phrase “PEG.sub.12000-IFN alpha” as used herein means conjugates such as are prepared according to the methods of International Application No. WO 95/13090 and containing urethane linkages between the interferon alpha-2a or alpha-2b amino groups and polyethylene glycol having an average molecular weight of 12000.

### DETAILED DESCRIPTION OF THE INVENTION

The compounds of the present invention can be readily prepared according to the following reaction schemes or modification thereof or otherwise using known chemistry procedures. In the following reaction schemes R₁, R₂, R₃, R₄, R₅, R₆, R₇, R₈, R₉, or R₁₀, R₁₁, R₁₂ and Y are selected from the groups defined above.

Preferred compounds of the present invention can be synthesized as described in the schemes below (Scheme 1 to 6).

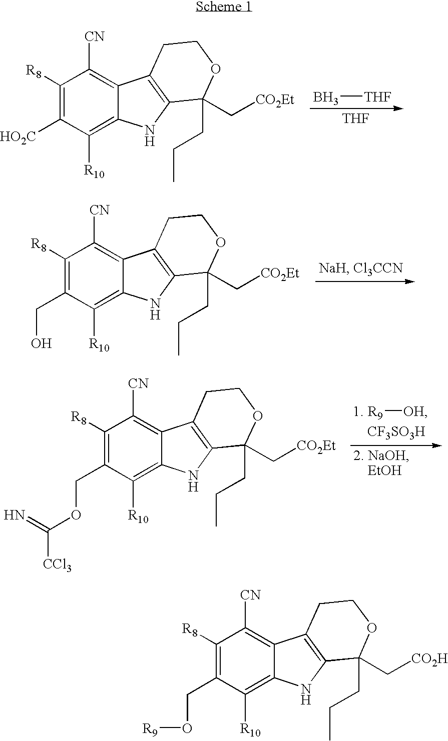

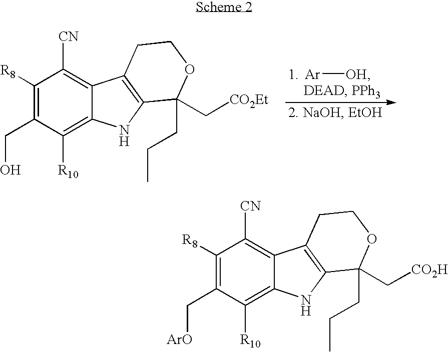

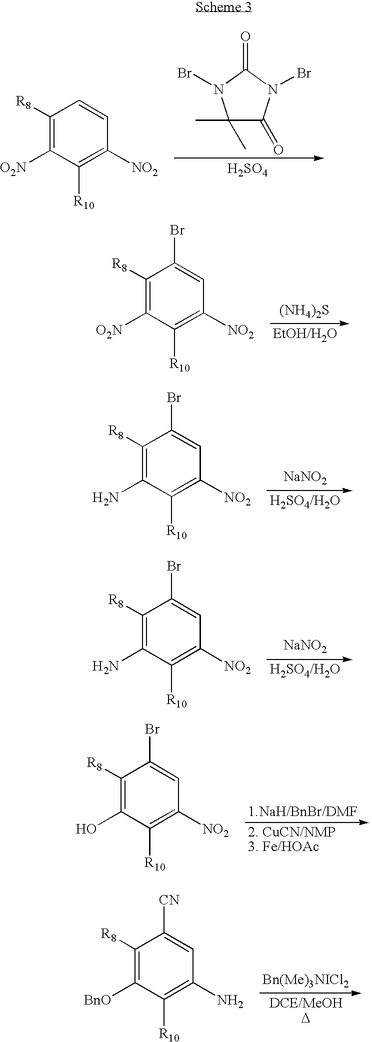

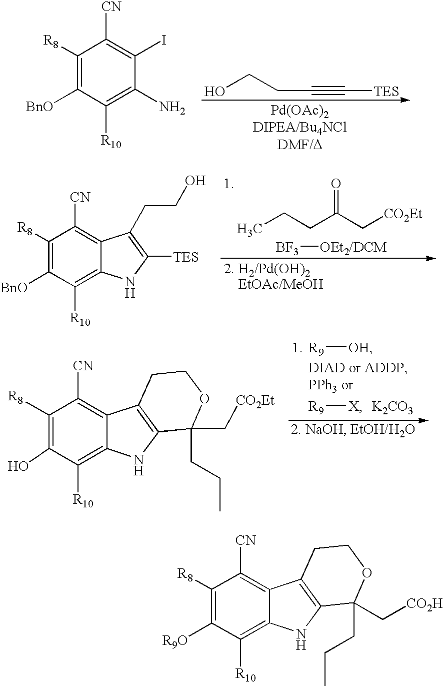

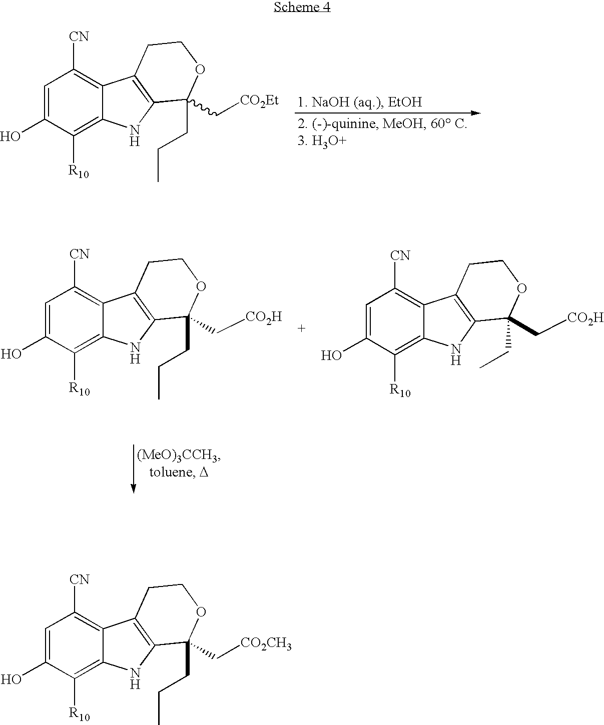

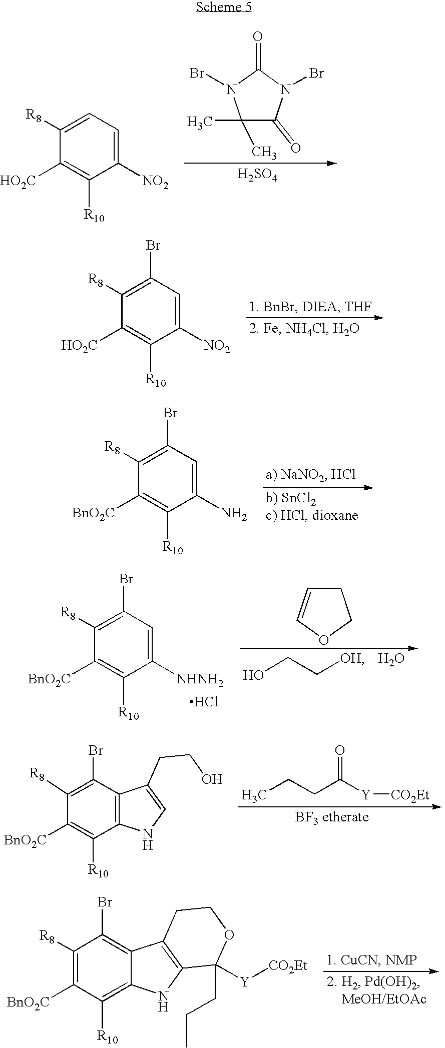

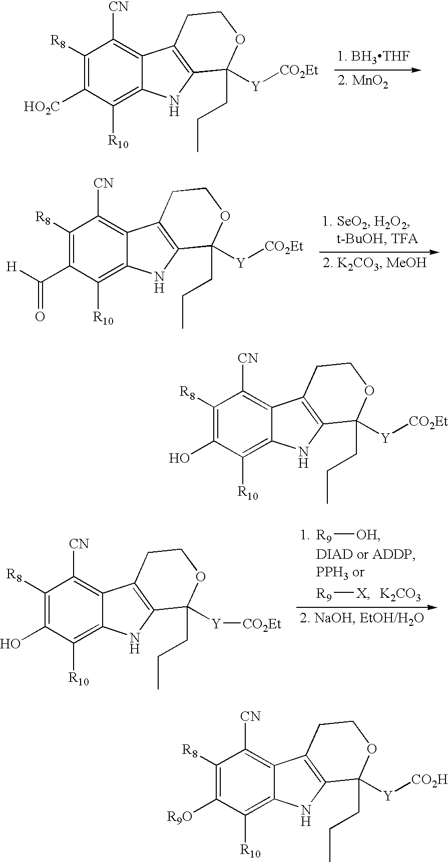

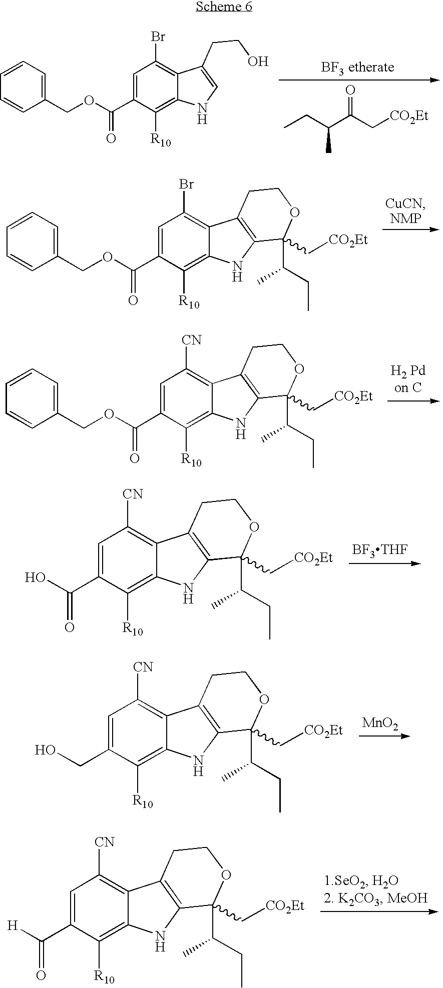

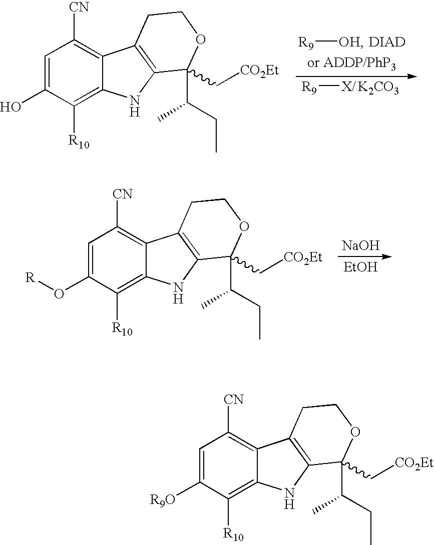

The ability of the compounds of the present invention to inhibit Hepatitis C Polymerase was established by the following experimental procedure:

NS5B from the BK strain (genotype 1b) is expressed in E coli as a protein in which the 21 C-terminal amino acids are replaced with a short linker and a hexahistidine tag (GSHHHHHH). The purified protein is mixed with radioactive nucleotides and allowed to replicate a heteropolymeric RNA substrate, primed by an endogenous short hairpin, resulting in an approximately 760 nt product. The radioactive product is captured on a filter and quantitated after removal of the unincorporated nucleotides.

Reagents:

10 mM uridine 5′-triphosphate (UTP) (Promega #p116B)

10 mM adenine 5′-triphosphate (ATP) (Promega #p113B)

10 mM cytidine 5′-triphosphate (CTP) (Promega #p114B)

10 mM guanine 5′-triphosphate (GTP) (Promega #p115B)

Bovine Serum Albumin (BSA) 10 mg/ml NEB (100× at 10 mg/ml) #007-BSA

RNasein (Promega #N251X) 40 U/μl

A-[33P]-GTP (NEN-easytides NEG/606H 3000 Ci/mmol, 370 MBq/ml, 10 mCi/ml)

Falcon polypropylene 96 well plates (Becton Dickinson #351190)

Millipore Multiscreen assay system-96 well-filtration plate #MADE NOB 50

Optiphase Supermix (Wallac) formulated by Fisher

Millipore Multiscreen liner for use in microbeta 1450-106 cassette [(Wallac) Perkin Elmer #1450-433]

1 M (N-[2-hydroxyethyl]piperazine-N′-[2-ethanesulfonic acid]) (HEPES), pH 7.3

Amersham Pharmacia Biotec (US16924-500 ml)

1 M MgCl₂ (SIGMA #M1028)

Dithiothreitol (DTT) (solid) (SIGMA #D9779)

RNase free water (GIBCO-BRL #10977-023)

Dimethyl sulfoxide (Aldrich #27685-5)

Basilen Blue (Sigma, B5520)

0.5M ethylenediaminetetraacetic acid (EDTA), pH 8 (GIBCO-BRL #15575-020)

Dibasic sodium phosphate (7-hydrate) (Na₂HPO₄.7H₂O; Baker#3824-07)

Phosphoric acid (Baker, #0262.02)

Further Reagent Preparation:

0.5M Na Phosphate buffer. Per liter, weigh 134 gr Na₂HPO₄.7H₂O, add water to 900 ml. Adjust pH to 7.0 with phosphoric acid. Top off with water to 1 L.
Dilute nucleotides 1:1000 to 10 μM (GTP and CTP) or 1:100 to 100 μM (ATP and UTP) into RNase free water.
   Procedure:
(1) Compounds 10 μl at 10 μg/ml in 15% dimethylsulfoxide (DMSO)
   When starting from 100 μg/ml compound stock in 1% DMSO:
   Dispense 5 μl 30% DMSO per well
   Dispense 5 μl compound (100 μg/ml) per well.
   When starting from 50 μg/ml compound stock in 15% DMSO:
   Add 10 μl compound per well.

(2) Enzyme Mix:

| | Final Conc (in 50 μl | | Per 20 μl mix (1 | | |
|---|---|---|---|---|---|
| Stock | assay volume) | | reaction) | Per 600 reactions | |
| Diethyl Pyrocarbonate (DEPC) | | | 17.06 μl | 10236 | μl |
| water | | | | | |
| 1 M HEPES, pH 7.5 | 20 | mM | 0.5 μl | 300 | μl |
| 1 M MgCl₂ | 5 | mM | 0.25 μl | 150 | μl |
| 100 mM DTT | 1 | mM | 0.5 μl | 300 | μl |
| 100 μM UTP | 0.5 | μM | 0.25 μl | 150 | μl |
| 100 μM ATP | 1 | μM | 0.5 μl | 300 | μl |
| 10 μM CTP | 0.08 | μM | 0.4 μl | 240 | μl |
| 10 μM GTP | 0.025 | μM | 0.125 μl | 75 | μl |
| BSA, 10 mg/ml | 0.05 | mg/ml | 0.25 μl | 150 | μl |
| HCV RdRp NS5B d21BK (500 | 24 | nM | 0.16 μl | 96 | μl |
| μg/ml or ˜7.5 μM) | | | | | |

| | | | |
|---|---|---|---|
| Total: | 20 μl | 12 | ml |

Add 20 μl enzyme mix into each well of the assay plate. Incubate compound and enzyme at room temperature for 15 minutes
(3) Template mix—prepare ahead

Spin down a tube of RNA (5 μg/tube stored in 75% ethanol and 0.3 M sodium acetate) in a microcentrifuge for 20 minutes at 4° C. One tube is enough for 1-1.5 plates. Remove as much ethanol from the tube as possible by inverting the tube. Be gentle, pellet RNA may not adhere to the tube. Vacuum dry the RNA. Resuspend the RNA by adding 1 ml of DEPC water, close the cap of the tube tightly. To dissolve RNA, incubate RNA solution on ice for ˜60 minutes and gently vortex. Spin briefly to ensure all RNA solution is down to the bottom of the tube before opening cap. Gently transfer RNA solution into a 5 ml or larger tube. Add another 3 ml of DEPC water (total 4 ml of volume).

Add the following volumes of reagents

| | Final | | Per 20 μl mix | Per 600 |
|---|---|---|---|---|
| Stock | concentration | | (1 reaction) | reactions |
| RNAse-free water | | | 2.98 μl | 1788 μl |
| HEPES, 1M | 20 | mM | 0.5 μl | 300 μl |
| RNase Inhibitor (40 U/μl) | 0.4 | μ/μl | 0.5 μl | 300 μl |
| 33P-GTP 3000 Ci/mmol, | 0.025 | μM | 0.0125 μl | 7.5 μl |
| 10 μCi/μl (3.3 μM) | | | | |
| POF RNA template | 3 | nM | 16 μl | 9600 μl |

Add 20 μl template mix per reaction (i.e. 20 ng of pOF per reaction or ˜3 nM)
(4) Incubate reaction at room temperature (22-25° C.) for 2 hours.
(5) Stop reaction by adding 50 μl of 170 mM EDTA.

Final concentration of EDTA is 85 mM.
(6) Prewet filters of Millipore multiscreen assay plate by adding 200 μl of 0.5 M sodium phosphate buffer, pH 7.0 into each well. Let stand at room temperature for 2-3 minutes.
(7) Place the multiscreen filter plate onto a Millipore Manifold and turn on vacuum to allow buffer to flow through. Turn off vacuum. Transfer 80 μl of the reaction product into each well of the filter plate. Let stand for 2-3 minutes. Turn on vacuum to filter reaction product.
(8) Turn off vacuum. Add 200 μl of 0.5 M sodium phosphate buffer, pH 7.0 into each well to wash filter. Turn on vacuum.

Repeat step (8) three more times.
(9) Remove polypropylene bottom. Spot dry filter at the bottom with paper towel. Air dry filter plate on a bench for 1 hour. Add 40 μl Super Mix scintillant. Seal top of the plate with a tape. Place plate into a Packard carrier or micro-beta carrier.
(10) Count plate using a Packard Topcount or micro-beta counter. Count (for example using Program 10) for ³³P in Top count or ³³P program in micro-beta.

See, Ferrari et al. 1999. J. Virology 73:1649-1654: “Characterization of soluble Hepatitis C virus RNA-dependent RNA polymerase expressed in E. coli and Takamizawa et al. 1991” and J. Virology 65:1105-1113: “Structure and characterization of the Hepatitis C virus genome isolated from human carriers,” both references are hereby incorporated by reference.

The compounds of the present invention inhibited Hepatitis C polymerase as summarized in Table 1:

TABLE 1

| | BK | BB7 |
|---|---|---|
| | IC₅₀ | IC₅₀ |
| | A = <0.5 μM, | A = <0.5 μM, |
| | B = 0.5 to <5.0 μM, | B = 0.5 to <5.0 μM, |
| Example | C = ≧5 μM | C = ≧5 μM |
| 1 | A | A |
| 2 | B | A |
| 3 | B | A |
| 4 | B | A |
| 5 | B | A |
| 6 | B | A |
| 7 | B | B |
| 8 | B | A |
| 9 | B | B |
| 10 | B | B |
| 11 | B | B |
| 12 | B | B |
| 13 | B | B |
| 14 | B | B |
| 15 | A | A |
| 16 | B | A |
| 17 | B | C |
| 18 | B | A |
| 19 | B | B |
| 20 | B | B |
| 21 | A | A |
| 22 | A | A |
| 23 | A | A |
| 24 | A | A |
| 25 | A | A |
| 26 | A | A |
| 27 | A | A |
| 28 | A | A |
| 29 | A | A |
| 30 | A | A |
| 31 | B | B |
| 32 | A | A |
| 33 | A | A |
| 34 | A | A |
| 35 | A | A |
| 36 | A | A |
| 37 | A | A |
| 38 | A | A |
| 39 | A | A |
| 40 | A | A |
| 41 | A | A |
| 42 | A | B |
| 43 | A | A |
| 44 | A | A |
| 45 | A | A |
| 46 | A | A |
| 47 | C | B |
| 48 | A | A |
| 49 | A | A |
| 50 | A | A |
| 51 | A | A |
| 52 | A | A |
| 53 | A | A |
| 54 | A | A |
| 55 | A | A |
| 56 | A | A |
| 57 | C | B |
| 58 | B | B |
| 59 | A | A |
| 60 | A | A |
| 61 | A | A |
| 62 | A | A |
| 63 | A | A |
| 64 | A | A |
| 65 | A | A |
| 66 | A | A |
| 67 | A | A |
| 68 | A | A |
| 69 | A | A |
| 70 | A | A |
| 71 | A | A |
| 72 | A | A |
| 73 | A | A |
| 74 | A | A |
| 75 | A | A |
| 76 | A | A |
| 77 | A | A |
| 78 | A | A |
| 79 | A | A |
| 80 | A | A |
| 81 | A | A |
| 82 | A | A |
| 83 | A | A |
| 84 | A | A |
| 85 | A | A |
| 86 | A | A |
| 87 | A | A |
| 88 | A | A |
| 89 | A | A |
| 90 | A | A |
| 91 | A | A |
| 92 | A | A |
| 93 | A | A |
| 94 | A | A |
| 95 | A | A |
| 96 | A | A |
| 97 | A | A |
| 98 | B | A |
| 99 | A | A |
| 100 | A | A |
| 101 | A | A |
| 102 | A | A |
| 103 | A | A |
| 104 | A | A |
| 105 | A | A |
| 106 | A | A |
| 107 | A | A |
| 108 | A | A |
| 109 | A | A |
| 110 | A | A |
| 111 | A | A |
| 112 | A | A |
| 113 | A | A |
| 114 | A | A |
| 115 | A | A |
| 116 | A | A |
| 117 | A | A |
| 118 | A | A |
| 119 | A | A |
| 120 | A | A |
| 121 | A | A |
| 122 | A | A |
| 123 | A | A |
| 124 | A | A |
| 125 | A | A |
| 126 | A | A |
| 127 | A | A |
| 128 | A | A |
| 129 | A | A |
| 130 | A | A |
| 131 | — | — |
| 132 | A | A |
| 133 | A | A |
| 134 | A | A |
| 135 | A | A |
| 136 | A | A |
| 137 | A | A |
| 138 | A | A |
| 139 | A | A |
| 140 | A | A |
| 141 | A | A |
| 142 | A | A |
| 143 | A | A |

the compounds of the present invention to inhibit Hepatitis C virus replicon constitutively expressed in a human liver cell line was established by the following experimental procedure:

Clone A cells (licensed from Apath, LLC) are derived from Huh-7 cells (human hepatoma cell line) and constitutively express the HCV replication proteins with concomitant amplification the HCV replicon (1b) genome. Cells are maintained and passaged in DMEM/10% FCS/1 mg/ml G418 (Geneticin from Gibco #11811-023; other media components as described below in “elisa media”). Care should be taken to maintain cell monolayers at a subconfluent state by 1:3 or 1:4 passages every 3-4 days. The replicon is extremely sensitive to the cellular metabolism/proliferation state and replicon copy number will rapidly decline in confluent monolayers (resting cells). Under ideal conditions each cell has, on average, 1000 copies of the HCV replicon genome.

Reagents:

Elisa Media:

Dulbecco's Modified Eagle Media (DMEM) (Gibco #12430-047)

2% Fetal Calf Serum (FCS) (HyClone #SH30070.03)

1× pen/strep (Gibco #15140-122)

1× Non-essential amino acids (NEAA) (Gibco #11140-050)

no G418

Glutaraldehyde (Fisher #02957-4)

TWEEN-20, 10% (Roche #1332465)

TRITON X-100 (Sigma #T-8787)

Superblock in Phosphate Buffered Saline (PBS) (Pierce #37515)

NS5a monoclonal antibody (Virostat #1873)

Goat antimouse-BRP monoclonal antibody (BioRad #172-1011)

3,3′,5,5′ tetramethylbenzidine (TMB) substrate (Sigma #T-0440)

Compound Dilution/Cell Plating:

Drug Plate Preparation (Mother Plate)

10 μl of compounds (in DMSO) are added to column 3 of the mother plate. 5 μl of DMSO are added to the remaining columns. Mother plates are set aside until ready for serial dilution to be performed.

Control Drugs

Drug and Cell Addition:

The process for each plate involves:

Prepare cell plates (daughter plates) by adding 52 μl of Elisa media to each well.

In Mother plates, serially transfer 50 μl/well from column 3 through column 12.

Transfer 8 μl from mother plate to daughter plates (all 96 wells).

Place daughter plates in incubator until cells are prepared.

Harvest Clone A cells and plate directly into daughter plates at 0.7×10⁵ cells/ml, 100 μl/well.

All plates are incubated at 37° C. in 5% CO₂ for 3 days.

Elisa Assay:

Remove media from 96-well plates (cells should be ca 80% confluent) by flicking into sink.

Add 130 μl/well 1×PBS+0.05% glutaraldehyde.

Incubate 37° C. for 1 hour.

Remove by flicking into sink.

Wash 3× with 300 μl/well PBS, shaking 5 min each wash. Remove by flicking into sink.

Add 130 μl/well PBS+0.05% TWEEN-20+0.1% TRITON X-100.

Incubate 37° C. for 10 minutes.

Remove by flicking into sink.

Add 300 μl/well Superblock in PBS.

Incubate 37° C. for 1 hour.

Remove by flicking into sink.

Wash 3× with 300 μl/well PBS, shaking 5 minutes each wash. Remove by flicking into sink.

During last wash, make a 1:100 dilution of NS5a Monoclonal-antibody (Mab) in Superblock+0.02% TWEEN-20.

After last wash, add 50 μl/well diluted Mab.

Incubate 37° C. for 1 hour.

Remove by flicking into sink.

Wash 3× with 300 μl/well PBS+0.02% TWEEN-20, shaking 5 minutes each wash.

Remove by flicking into sink.

During last wash, make a 1:500 dilution of goat antimouse-BRP Mab in Superblock+0.02% TWEEN-20.

After last wash, add 50 μl/well diluted Mab.

Incubate 37° C. for 1 hour.

Remove by flicking into sink.

Wash 5× with 300 μl/well PBS+0.02% TWEEN-20, shaking 5 minutes each wash. Remove by flicking into sink.

Wash 3× with 300 μl/well PBS, shaking 5 minutes each wash. Remove by flicking into sink.

After last wash, add 130 μl/well room temperature TMB substrate.

Incubate until blue color develops.

Add 130 μl/well 1N HCl to stop reaction (color turns from blue to yellow).

Read plates with optical density (O.D.) 450 filter.

ANALYSIS OF RESULTS: IC₅₀ (ELM); IC₅₀ (μg/ml); % Inhibition

REFERENCE COMPOUNDS: Interferon-a₂; 4-30 U/ml IC50

The following non-limiting specific examples are included to illustrate the synthetic procedures used for preparing compounds of the Formula I. In these examples, all chemicals and intermediates are either commercially available or can be prepared by standard procedures found in the literature or are known to those skilled in the art of organic synthesis.

### EXAMPLE 1

### 5-Bromo-1-ethoxycarbonylmethyl-8-methyl-1-propyl-1,3,4,9-tetrahydro-pyrano[3,4-b]indole-7-carboxylic acid benzyl ester

A 1000 mL round-bottom flask (RBF) was charged with of 4-bromo-3-(2-hydroxy-ethyl)-7-methyl-1H-indole-6-carboxylic acid benzyl ester (12.03 g, 0.031 mol), ethyl butyrylacetate (5.45 mL, 0.034 mol), and 500 mL dichloromethane (DCM). This mixture was cooled to 0° C. with stirring. Over approximately 5 minutes boron trifluoride diethyl etherate (BF₃Et₂O) (9.82 mL, 0.078 mol) was added to the stirring mixture. The reaction was allowed to warm to ambient temperature and was stirred for 1 hour. The reaction was then diluted with ethyl acetate (EtOAc), washed saturated NaHCO₃ (2×) and saturated NaCl (1×), dried (MgSO₄), and concentrated. The resulting yellow oil was purified by flash chromatography on SiO₂ eluting with a 10% EtOAc/hexanes to 20% EtOAc/hexanes gradient. Trituration in hexanes yielded 13.71 g (84.0%) of white powder. ¹H NMR (CDCl₃) δ 9.81 (br s, 1H), 7.89 (s, 1H), 7.40 (m, 5H), 5.35 (s, 2H), 4.21 (m, 2H), 3.95 (m, 2H), 3.15 (t, J=4.7 Hz, 2H), 3.00 (m, J=22.3 Hz, 2H), 2.73 (s, 3H), 2.00 (m, 2H), 1.29 (m, 4H), 0.88 (t, J=7.6 Hz, 3H).

### 5-Cyano-1-ethoxycarbonylmethyl-8-methyl-1-propyl-1,3,4,9-tetrahydro-pyrano[3,4-b]indole-7-carboxylic acid benzyl ester

A 500 mL RBF was charged with 5-bromo-1-ethoxycarbonylmethyl-8-methyl-1-propyl-1,3,4,9-tetrahydro-pyrano[3,4-b]indole-7-carboxylic acid benzyl ester (12.00 g, 23 mmol), 100 mL 1-methyl-2-pyrrolidinone (NMP), and CuCN (20.34 g, 0.227 mol). The reaction was heated to 190° C. with vigorous stirring for 45 minutes. The reaction was then cooled to ambient temperature and diluted with EtOAc. Water (300 mL) was added to the mixture, followed by the addition of a 1:1 mixture of Celite/silica gel. This suspension was stirred for several minutes, and then filtered through a pad of Celite. The Celite washed with EtOAc, and the filtrate washed H₂O (5×) and saturated NaCl (1×), dried over MgSO₄, and concentrated. Upon trituration with hexanes, 8.82 g (81.8%) of light tan solid was obtained. ¹H NMR (CDCl₃) δ 10.11 (br s, 1H), 8.12 (s, 1H), 7.40 (m, 5H), 5.37 (s, 2H), 4.22 (m, 2H), 3.95 (m, 2H), 3.00 (m, 4H), 2.83 (s, 3H), 2.00 (m, 2H), 1.30 (m, 4H), 0.89 (t, J=7.6 Hz, 3H).

### 5-Cyano-1-ethoxycarbonylmethyl-8-methyl-1-propyl-1,3,4,9-tetrahydro-pyrano[3,4-b]indole-7-carboxylic acid

A 500 mL Parr flask was charged with 5-cyano-1-ethoxycarbonylmethyl-8-methyl-1-propyl-1,3,4,9-tetrahydro-pyrano[3,4-b]indole-7-carboxylic acid benzyl ester (14.24 g, 0.030 mol), 180 mL of 1:1 MeOH/EtOAc, and Pd(OH)₂ (20% on C, 3.02 g). The flask was shaken on a Parr shaker at 5 psi above ambient pressure for 30 minutes. The reaction mixture was filtered through a pad of Celite, washed with methanol and concentrated. Trituration with hexanes yielded 11.1 g (96.4%) of off-white powder. ¹H NMR (CDCl₃) δ 10.21 (br s, 1H), 8.22 (s, 1H), 4.22 (m, 2H), 4.00 (m, 2H), 3.09 (m, 4H), 2.89 (s, 3H), 2.00 (m, 2H), 1.30 (m, 4H), 0.90 (t, J=7.6 Hz, 3H).

### (5-Cyano-7-hydroxymethyl-8-methyl-1-propyl-1,3,4,9-tetrahydro-pyrano[3,4-b]indol-1-yl)-acetic acid ethyl ester

To a solution of 5-cyano-1-ethoxycarbonylmethyl-8-methyl-1-propyl-1,3,4,9-tetrahydro-pyrano[3,4-b]indole-7-carboxylic acid (11.1 g, 29.0 mmol), which can be prepared according to the previous step, in tetrahydrofuran (THF) (150 mL) at 0° C. was added BH₃.THF (1.0M in THF, 72.2 mL, 72.2 mmol). The cooling bath was removed, and the solution was stirred for 1.5 hours. The reaction was quenched with 3% HCl, diluted with EtOAc, and the layers were separated. The organic layer washed with saturated NaHCO₃, H₂O, and brine, dried (MgSO₄) and concentrated in vacuo. Purification via flash chromatography on SiO₂ using 20% ethyl acetate/dichloromethane (EtOAc/DCM) as eluent afforded 8.28 g (77%) of pale green foam. ESI-MS m/z 369 (M−H)−. ¹H NMR (CDCl₃) δ 9.72 (bs, 1H), 7.43 (s, 1H), 4.80 (d, J=5.5 Hz, 2H), 4.25 (m, 2H), 4.15 (m, 1H), 3.93 (m, 1H), 3.02 (m, 3H), 2.92 (d, J=17.0 Hz, 1H), 2.57 (s, 3H), 2.00 (m, 2H), 1.30 (m, 5H), 0.88 (t, J=7.6 Hz, 3H).

### EXAMPLE 2

### (5-Cyano-7-methoxymethyl-8-methyl-1-propyl-1,3,4,9-tetrahydro-pyrano[3,4-b]indol-1-yl)-acetic acid

### [5-Cyano-8-methyl-1-propyl-7-(2,2,2-trichloro-acetimidoyloxymethyl)-1,3,4,9-tetrahydro-pyrano[3,4-b]indol-1-yl]-acetic acid ethyl ester

To a solution of (5-cyano-7-hydroxymethyl-8-methyl-1-propyl-1,3,4,9-tetrahydro-pyrano[3,4-b]indol-1-yl)-acetic acid ethyl ester (0.75 g, 2.0 mmol) in DCM (12 mL) was added sodium hydride (9.7 mg, 4.0 mmol). After 10 minutes, trichloroacetonitrile (608 μL, 6.0 mmol) was added. The reaction was stirred for 4 hours, at which point additional trichloroacetonitrile (200 mL) was added. After an additional 45 minutes of stirring, sodium hydride (4 mg) was added. The reaction was placed in a freezer at 0° C. overnight. After warming to room temperature the next morning, an additional aliquot of sodium hydride (9.7 mg) was added. The reaction was stirred for 1.5 hours, quenched with H₂O and diluted with EtOAc. The layers were separated, and the organic layer washed with brine, dried (MgSO₄) and concentrated to afford 1.14 g (109%) of an off-white solid which was used without further purification. ¹H NMR (CDCl₃) δ 9.84 (s, 1H), 8.43 (s, 1H), 7.53 (s, 1H), 5.44 (s, 2H), 4.22 (m, 2H), 4.090 (m, 1H), 3.95 (m, 1H), 3.07 (m, 3H), 2.96 (d, J=15.2 Hz, 1H), 2.61 (s, 3H), 2.06 (m, 1H), 1.95 (m, 1H), 1.42 (m, 1H), 1.12 (m, 4H), 0.91 (t, J=7.3 Hz, 3H).

### (5-Cyano-7-methoxymethyl-8-methyl-1-propyl-1,3,4,9-tetrahydro-pyrano[3,4-b]indol-1-yl)-acetic acid

To a solution of crude [5-cyano-8-methyl-1-propyl-7-(2,2,2-trichloro-acetimidoyloxymethyl)-1,3,4,9-tetrahydro-pyrano[3,4-b]indol-1-yl]-acetic acid ethyl ester (45.7 mg, 0.089 mmol) in DCM/cyclohexanol (0.5 mL/0.5 mL) at 0° C. was added methanol (14 μL, 0.35 mmol) followed by trifluoromethanesulfonic acid (2 mL, 0.022 mmol). The cold bath was removed, and the solution stirred at ambient temperature for 20 minutes. The reaction was quenched by the addition of saturated NaHCO₃ and extracted with EtOAc. The organic layer was washed with H₂O and brine, dried (Na₂SO₄) and concentrated. The crude material was chromatographed on SiO₂ with 0.5% diethyl ether in DCM to 4% diethyl ether/DCM gradient to afford 13.3 mg (53%) of a colorless oil. ¹H NMR (CDCl₃) δ 9.67 (s, 1H), 7.40 (s, 1H), 4.55 (s, 2H), 4.21 (m, 2H), 4.05 (m, 1H), 3.91 (m, 1H), 3.34 (s, 3H), 3.06 (m, 3H), 2.91 (d, J=16.4 Hz, 1H), 2.53 (s, 3H), 2.11 (m, 1H), 1.95 (m, 1H), 1.29 (m, 5H), 0.88 (t, J=7.3 Hz, 3H).

The ethyl ester was saponified using 10% NaOH (aq., 150 μL) in methanol (2 mL) overnight to afford 8.8 mg (71%) of the carboxylic acid. ESI-MS m/z 355 (M−H)−; ¹H NMR (CD₃OD) δ 10.71 (s, 1H), 7.38 (s, 1H), 4.58 (s, 2H), 4.03 (m, 2H), 3.39 (s, 3H), 3.03 (d, J=14.1 Hz, 1H), 2.98 (m, 2H), 2.85 (d, J=14.1 Hz, 1H), 2.56 (s, 3H), 2.04 (m, 2H), 1.42 (m, 1H), 1.01 (m, 1H), 0.87 (t, J=7.3 Hz, 3H).

Examples 3-19 were synthesized following the above mentioned procedure for example 1 using the intermediate [5-cyano-8-methyl-1-propyl-7-(2,2,2-trichloro-acetimidoyloxymethyl)-1,3,4,9-tetrahydro-pyrano[3,4-b]indol-1-yl]-acetic acid ethyl ester and coupling it with the following alcohols: ethanol, 1-propanol, 2-propanol, cyclobutanol, cyclohexanol, cyclopropanemethanol, cyclobutanemethanol, cyclopentanemethanol, 2-butyn-1-ol, tetrahydro-4H-pyran-4-ol, (S)-3-hydroxytetrahydrofuran, (R)-3-hydroxytetrahydrofuran, benzyl alcohol, piperonyl alcohol, 2,4-dimethylbenzyl alcohol, 3-thiophenemethanol, and 2,4-dimethylthiazole-5-methanol. The resulting esters were hydrolyzed using 10% NaOH (aq) in ethanol (EtOH).

### EXAMPLE 20

### (5-Cyano-8-methyl-7-phenoxymethyl-1-propyl-1,3,4,9-tetrahydro-pyrano[3,4-b]indol-1-yl)-acetic acid

To a solution of (5-cyano-7-hydroxymethyl-8-methyl-1-propyl-1,3,4,9-tetrahydro-pyrano[3,4-b]indol-1-yl)-acetic acid ethyl ester (42 mg, 0.11 mmol) in THF (1.1 mL) was added triphenylphosphine (42 mg, 0.16 mmol) and phenol (15.3 mg, 0.16 mmol). Diethylazodicarboxylate (DEAD, 26 μL, 0.16 mmol) was added via syringe, and the reaction was stirred for 1.5 hours, at which point additional portions of DEAD and triphenylphosphine were added. The reaction was stirred for an additional 1.5 hours. EtOAc and 10% NaOH were added, and the layers were separated. The organic layer washed 10% NaOH (2×), 3% HCl (1×), saturated NaHCO₃ (1×), and brine; dried (Na₂SO₄) and concentrated. Flash chromatography of the crude product on SiO₂ with 5% EtOAC/hexane to 15% EtOAc/hexane gradient afforded 16.3 mg (32%) of a colorless solid. ¹H NMR (CDCl₃) δ 9.76 (s, 1H), 7.50 (s, 1H), 7.3 (m, 3H), 6.98 (m, 2H), 5.13 (s, 2H), 4.26 (m, 2H), 4.12 (m, 1H), 3.95 (m, 1H), 3.08 (m, 2H), 3.01 (d, J=17.0 Hz, 1H), 2.92 (d, J=17.0 Hz, 1H), 2.56 (s, 3H), 2.04 (m, 1H), 1.97 (m, 1H), 1.3 (m, 5H), 0.89 (t, J=7.3 Hz, 3H).

The ethyl ester was saponified using 10% NaOH (aq., 150 μL) in ethanol (1.5 mL) overnight to afford 15.7 mg (99%) of the carboxylic acid. ESI-MS m/z 417 (M−H)−; ¹H NMR (d₆-DMSO) δ 11.16 (br s, 1H), 7.58 (s, 1H), 7.32 (m, 2H), 7.05 (m, 2H), 6.96 (t, J=7.3 Hz, 1H), 5.20 (s, 2H), 3.98 (m, 2H), 2.98 (d, J=13.5 Hz, 1H), 2.88 (m, 2H), 2.75 (d, J=13.5 Hz, 1H), 2.57 (s, 3H), 2.00 (m, 2H), 1.25 (m, 1H), 0.80 (m, 4H).

Example 21 was prepared using the above mentioned procedure for Example 20 using the intermediate (5-cyano-7-hydroxymethyl-8-methyl-1-propyl-1,3,4,9-tetrahydro-pyrano[3,4-b]indol-1-yl)-acetic acid ethyl ester and coupling it with 3-fluorophenol. The resulting ester was hydrolyzed using 10% NaOH (aq) in EtOH.

### EXAMPLE 22

### (5-Cyano-7-cyclopropylmethoxy-8-methyl-1-propyl-1,3,4,9-tetrahydro-pyrano[3,4-b]indol-1-yl)-acetic acid

### 5-Bromo-2-methyl-1,3-dinitro-benzene

2,6-Dinitrotoluene (1.0 g, 5.5 mmol) was suspended in 4 mL con. H₂SO₄. 1,3-Dibromo-5,5-dimethylhydantoin (0.86 g, 3 mmol) was added portionwise to the mixture over 10 minutes. After a slight exotherm, the mixture went into solution briefly, and then a precipitate formed. The mixture was stirred at room temperature for 1 hour, and then the solid was filtered off. The pale yellow solid was dried on a vacuum pump to give 1.33 g (92%) of the product. Purity 94% (GC); MS 260, 262 m/z (Br pattern); ¹H NMR (CDCl₃) δ 8.13 (s, 2H), 2.52 (s, 3H).

### 5-Bromo-2-methyl-3-nitro-phenylamine

5-Bromo-2-methyl-1,3-dinitro-benzene (1.0 g, 3.8 mmol) was mixed with EtOH (23 mL) and pyridine (1.56 mL, 19.0 mmol) and heated to reflux. A 20% solution of ammonium sulfide (3.89 g) in water was further diluted with 4 mL water then added via an addition funnel to the refluxing mixture over 1 hour. After the addition was complete, the reflux was continued for 2 hours. The reaction was cooled to room temperature, then poured onto a 1:1 mixture of water and ice (200 mL). The bright yellow precipitated solid was filtered off and dried on a vacuum pump to give 0.73 g (83%) of the product. Purity 100% (GC); MS 230, 232 m/z (Br pattern); ¹H NMR (CDCl₃). δ 7.29 (d, J=1.7 Hz, 1H), 6.99 (d, J=1.7 Hz, 1H), 3.97 (bs, 2H), 2.18 (s, 3H).

### 5-Bromo-2-methyl-3-nitro-phenol

5-Bromo-2-methyl-3-nitro-phenylamine (5.0 g, 21.5 mmol) was suspended in a solution of conc. H₂SO₄ (6.4 mL) and water (21.2 mL). The mixture was cooled with an ice bath to ˜0° C., then a solution of sodium nitrite (1.66 g, 24.0 mmol) in water (6.4 mL) was added via an addition funnel at a rate to maintain the temperature below 10° C. This mixture was stirred at this temperature for 1 hour. The mixture was transferred via a plastic canula to a solution of conc. H₂SO₄ (21.2 mL) and water (14.9 mL) heated at 130-150° C. The internal temperature of the mixture dropped to ˜80° C. during the addition. The mixture was heated until the internal temperature returned to at least 110° C. The mixture was cooled slightly then poured on ice (200 mL), followed by an extraction with t-butylmethyl ether (3×100 mL). The organic layer was dried over MgSO₄, filtered, and solvent removed under reduced pressure to give 3.61 g (72%) of the product as a dark orange-red solid. Purity 98.5% (GC); MS 231, 233 m/z (Br pattern); ¹H NMR (CDCl₃) δ 10.82 (s, 1H), 7.53 (d, J=1.7 Hz, 1H), 7.25 (d, J=1.7 Hz, 1H), 2.17 (s, 3H).

### 1-Benzyloxy-5-bromo-2-methyl-3-nitro-benzene

To a slurry of NaH (767 mg, 32 mmol) in DMF (10 mL) was added a solution of 5-bromo-2-methyl-3-nitro-phenol (6.4 g, 27 mmol) in DMF (10 mL) at 0° C. After 15 minutes, the solution was treated with benzyl bromide (3.4 mL, 28 mmol) dropwise. The reaction mixture was warmed to room temperature. After 16 hours, the solution was carefully diluted with brine and extracted with diethyl ether (3×). The combined organics were dried over Na₂SO₄, filtered and concentrated. The crude material was purified by flash chromatography (SiO₂, 6/1 hexanes/EtOAc) to give 5.4 g (62%) of a tan-orange solid. GCMS M⁺ 321 m/z. ¹H NMR (CDCl₃) δ 7.57 (s, 1H), 7.42-7.36 (m, 6H), 5.10 (s, 2H), 2.35 (s, 3H).

### 3-Benzyloxy-4-methyl-5-nitro-benzonitrile

A solution of 1-benzyloxy-5-bromo-2-methyl-3-nitro-benzene (11 g, 34 mmol) in NMP (40 mL) was treated with CuCN (18 g, 202 mmol) at room temperature. This solution was heated at 180° C. for 1 hour. The solution was then cooled to room temperature, diluted with H₂O and EtOAc and filtered through a Celite/silica gel pad. The filter cake was thoroughly rinsed with EtOAc. The filtrate was extracted with EtOAc and brine (2×), dried over Na₂SO₄, filtered and concentrated. The residual oil was diluted with diethyl ether and washed with brine, dried over Na₂SO₄, filtered and concentrated. Purification of the residual oil by flash chromatography (SiO₂, 5/1 hexanes/EtOAc) followed by trituration with Et₂O/hexanes provided 3.9 g (43%) of the benzonitrile as an off-white solid in addition to 3 g (37%) of the cyano-aniline as a light tan solid. GCMS M⁺ 268 m/z. ¹H NMR (CDCl₃) δ 7.69 (s, 1H), 7.43-7.39 (m, 6H), 5.17 (s, 2H), 2.47 (s, 3H).

### 3-Amino-5-benzyloxy-4-methyl-benzonitrile

A solution of 3-benzyloxy-4-methyl-5-nitro-benzonitrile (3.9 g, 14.5 mmol) in EtOH (40 mL) was treated with Fe powder (2.5 g) followed by acetic acid (4.0 mL) at room temperature. The solution was then heated at reflux for 2 hours. The solution was cooled to room temperature, filtered through a pad of Celite/silica gel and rinsed with EtOAc. The filtrate was concentrated in vacuo, extracted with EtOAc and saturated NaHCO₃ (2×), washed with brine, dried over Na₂SO₄, filtered and concentrated to give 3.3 g (96%) of a light tan solid. GCMS M⁺ 238 m/z. ¹H NMR (CDCl₃) δ 7.43-7.32 (m, 6H), 6.62 (s, 1H), 5.04 (s, 2H), 3.80 (bs, 2H), 2.11 (s, 3H).

### 3-Amino-5-benzyloxy-2-iodo-4-methyl-benzonitrile

A solution of 3-amino-5-benzyloxy-4-methyl-benzonitrile (4.2 g, 22 mmol) in dichloroethane (40 mL) and methanol (20 mL) was treated with CaCO₃ (9 g, 90 mmol) followed by benzyltrimethylammonium dichloroiodate (9.2 g, 26 mmol) at room temperature. This solution was heated at reflux for 7 hours. The reaction was then cooled to room temperature, filtered through a Celite/silica gel pad and thoroughly rinsed with EtOAc. The filtrate was extracted (2×) with 10% NaHSO₃, brine, dried over Na₂SO₄, filtered and concentrated. The residue was purified by flash chromatography (SiO₂, 4/1 hexanes/EtOAc) followed by trituration with diethyl ether/hexanes to give 5.9 g (86%) of a light tan solid. GCMS M⁺ 364 m/z. ¹H NMR (CDCl₃) δ 7.41-7.35 (m, 5H), 6.71 (s, 1H), 5.04 (s, 2H), 4.36 (bs, 2H), 2.20 (s, 3H).

### 6-Benzyloxy-3-(2-hydroxy-ethyl)-7-methyl-2-triethylsilanyl-1H-indole-4-carbonitrile

A solution of 4-triethylsilanyl-but-3-yn-1-ol (913 mg, 4.9 mmol) in DMF (2 mL) was treated with 3-amino-5-benzyloxy-2-iodo-4-methyl-benzonitrile (497 mg, 1.4 mmol) in DMF (2 mL) followed by tetrabutylammonium chloride (401 mg, 1.4 mmol), triphenylphosphine (115 mg, 0.43 mmol), palladium acetate (90 mg, 0.40 mmol), and diisopropylethylamine (1.0 mL, 5.7 mmol) at room temperature. This solution was heated at 85° C. for 3.5 hours. After cooling the reaction to room temperature, the reaction mixture was filtered through a Celite/silica gel pad. The filter pad was thoroughly washed with EtOAc. The filtrate was extracted with brine (2×), dried over Na₂SO₄, filtered, and concentrated. The residue was diluted with diethyl ether and washed with brine (2×), dried over Na₂SO₄, filtered, and concentrated. The crude oil was purified by flash chromatography (SiO₂, 4/1-1/1 hexanes/EtOAc) to give 422 mg (72%) of an orange oil that solidified upon standing. LCMS (M−H) 419 m/z. ¹H NMR (CDCl₃) δ 7.99 (s, 1H), 7.47-7.34 (m, 5H), 7.17 (s, 1H), 5.12 (s, 2H), 3.95-3.91 (m, 2H), 3.33-3.28 (m, 2H), 2.47(s, 3H), 2.12-2.09 (m, 1H), 1.05-0.92 (m, 15H).

### (±)-(7-Benzyloxy-5-cyano-8-methyl-1-propyl-1,3,4,9-tetrahydro-pyrano[3,4,-b]indol-1-yl)-acetic acid ethyl ester

A solution of 6-benzyloxy-3-(2-hydroxy-ethyl)-7-methyl-2-triethylsilanyl-1H-indole-4-carbonitrile (242 mg, 0.58 mmol) in DCM (4 mL) was treated with BF₃—OEt₂ (0.08 mL, 0.64 mmol) at room temperature. After 0.5 hours, additional BF₃—OEt₂ (0.10 mL) was added. After 0.5 hours, ethyl butyrlacetate (0.12 mL, 0.75 mmol) was added. After 3 hours, the reaction was diluted with brine, extracted with EtOAc (3×), dried over Na₂SO₄, filtered and concentrated. The residue was triturated with diethyl ether/hexanes to give 130 mg (50%) of the pyran as an off-white solid. LCMS (M−H) 445 m/z. ¹H NMR (CDCl₃) δ 9.44 (s, 1H), 7.45-7.33 (m, 5H), 7.10 (s, 1H), 5.11 (s, 2H), 4.29-4.11 (m, 2H), 4.08-4.02 (m, 1H), 3.95-3.79 (m, 1H), 3.06-3.02 (m, 2H), 2.96 (q, J_{AB}=18 Hz, 2H), 2.43 (s, 3H), 2.12-1.83 (m, 2H), 1.42-1.33 (m, 1H), 1.29 (t, J=6.9 Hz, 3H), 1.23-1.14 (m, 1H), 0.88 (t, J=7.2 Hz, 3H).

### (±)-(5-Cyano-7-hydroxy-8-methyl-1-propyl-1,3,4,9-tetrahydro-pyrano[3,4-b]indol-1-yl)-acetic acid ethyl ester

A solution of (±)-(7-benzyloxy-5-cyano-8-methyl-1-propyl-1,3,4,9-tetrahydro-pyrano[3,4,-b]indol-1-yl)-acetic acid ethyl ester (143 mg, 0.32 mmol) in EtOAc (10 mL) and MeOH (10 mL) was treated with Pd(OH)₂ (20 mg) and subjected to H₂ using Parr shaker. After 1 hour, the solution was filtered through a Celite plug. The filter pad was thoroughly rinsed with EtOAc. The filtrate was concentrated, and the residue was then triturated with diethyl ether/hexanes to give the phenol as an off-white solid (69 mg, 61%). ¹H NMR (CDCl₃) δ 9.45 (s, 1H), 6.94 (s, 1H), 4.65 (s, 1H), 4.26-4.11 (m, 2H), 4.07-4.00 (m, 1H), 3.94-3.86 (m, 1H), 3.04-3.01 (m, 2H), 2.96 (q, J_{AB}=17 Hz, 2H), 2.41 (s, 3H), 2.12-2.01 (m, 1H), 1.96-1.86 (m, 1H), 1.43-1.33 (m, 1H), 1.29 (t, J=7.5 Hz, 3H), 1.23-1.17 (m, 1H), 0.88 (t, J=7.5 Hz, 3H).

### (5-Cyano-7-cyclopropylmethoxy-8-methyl-1-propyl-1,3,4,9-tetrahydro-pyrano[3,4-b]indol-1-yl)-acetic acid

To a solution of (5-cyano-7-hydroxy-8-methyl-1-propyl-1,3,4,9-tetrahydro-pyrano[3,4-b]indol-1-yl)-acetic acid ethyl ester (36.8 mg, 0.10 mmol) in THF (2 mL) was added triphenyl phosphine (52 mg, 0.20 mmol) and cyclopropanemethanol (16 μL, 0.2 mmol). Diisopropylazodicarboxylate (DIAD, 39 μL, 0.2 mmol) was added. The reaction was stirred for 20 minutes, then quenched with H₂O and diluted with EtOAc. The organic layer washed with H₂O (1×) and brine, dried (Na₂SO₄) and concentrated. Flash chromatography on SiO₂ with 8% EtOAc/hexane afforded 29.5 mg (70%) of the product as a white solid. ¹H NMR (CDCl₃) δ 9.43 (br s, 1H), 7.03 (s, 1H), 4.21 (m, 2H), 4.07 (m, 1 h), 3.9 (m, 3H), 3.02 (m, 3H), 2.90 (d, J=16.4 Hz), 1H), 2.43 (s, 3H), 2.03 (m, 1H), 1.96 (m, 1H), 1.3 (m, 5H), 0.88 (t, J=7.3 Hz), 0.64 (m, 2H), 0.37 (m, 2H).

To a solution of the ethyl ester (29.4 mg, 0.072 mmol) in EtOH (2 mL) and THF (0.15 mL) was added 10% NaOH (aq., 0.2 mL). After stirring for 17.25 hours, the solution was acidified with 3% HCl and extracted with EtOAc. The organic layer washed with H₂O and brine, dried (Na₂SO₄) and concentrated to afford 26.9 mg (98%) of the title compound as a white solid. ESI-MS m/z 383 (MH)+. ¹H NMR (CD₃OD) δ 7.07 (s, 1H), 4.05 (m, 2H), 3.87 (d, J=6.4 Hz), 2.99 (m, 3H), 2.83 (d, J=14.1 Hz, 1H), 2.43 (s, 3H), 2.02 (m, 2H), 1.40 (m, 2H), 1.02 (m, 1H), 0.86 (t, J=7.3 Hz, 3H), 0.61 (m, 2H), 0.36 (m, 2H).

### EXAMPLE 23

### (R)-[5-Cyano-8-methyl-7-(5-methyl-isoxazol-3-ylmethoxy)-1-propyl-1,3,4,9-tetrahydro-pyrano[3,4-b]indol-1-yl]-acetic acid

### (R)-(5-Cyano-7-hydroxy-8-methyl-1-propyl-1,3,4,9-tetrahydro-pyrano[3,4-b]indol-1-yl)-acetic acid ethyl ester

Preparative chiral HPLC using CHIRALPAK AS (250×20 mm) and 20% 2-propanol in heptane as eluant gave the (R) and (S) enantiomers of (5-cyano-7-hydroxy-8-methyl-1-propyl-1,3,4,9-tetrahydro-pyrano[3,4-b]indol-1-yl)-acetic acid ethyl ester as white solids. Analytical chiral HPLC HP 100 with CHIRALPAK AS, 250×4.6 mm, 2-propanol/heptane (20/80), 1.0 mL/min, 235 nm UV detection; tR=5.70 min (R enantiomer), 12.74 min (S enantiomer).

### (R)-[5-Cyano-8-methyl-7-(5-methyl-isoxazol-3-ylmethoxy)-1-propyl-1,3,4,9-tetrahydro-pyrano[3,4-b]indol-1-yl]-acetic acid

To a solution of (R)-(5-cyano-7-hydroxy-8-methyl-1-propyl-1,3,4,9-tetrahydro-pyrano[3,4-b]indol-1-yl)-acetic acid ethyl ester (0.17 g, 0.47 mmol) in dry DMF (3 mL) was added milled potassium carbonate (71 mg, 0.52 mmol). This was stirred for 15 minutes, at which point 3-bromomethyl-5-methyl-isoxazole (91 mg, 0.52 mmol) was added. The reaction was stirred for 16 hours at ambient temperature, diluted with H₂O and extracted with EtOAc. The organic layer washed several times with H₂O and once with brine, dried (MgSO4), and concentrated. The residue was flash chromatographed on SiO₂ using 20% EtOAc in hexanes resulting in 185 mg (88% yield) of the product as an oil which crystallized upon standing.

To a solution of (R)-[5-cyano-8-methyl-7-(5-methyl-isoxazol-3-ylmethoxy)-1-propyl-1,3,4,9-tetrahydro-pyrano[3,4-b]indol-1-yl]-acetic acid ethyl ester (185 mg, 0.40 mmol) in ethanol (10 mL) was added 2M sodium hydroxide (0.4 mL). After stirring for 36 hours, the reaction was concentrated in vacuo. The residue was taken up in water and extracted with EtOAc. The aqueous layer was acidified and re-extracted with EtOAc. The combined organic layers were dried (MgSO₄) and concentrated to afford 150 mg (92%) of the title compound. ESI-MS m/z 424 (MH)+. ¹H NMR (CD₃OD) δ 7.20 (s, 1H), 6.25 (s, 1H), 5.15 (s, 2H), 3.0 (m, 3H), 2.83 (d, J=14.1 Hz, 1H), 2.43 (s, 3H), 2.42 (s, 3H), 2.04 (m, 2H), 1.40 (m, 1H), 1.01 (m, 1H), 0.86 (t, J=7.3 Hz, 3H).

### EXAMPLE 24

### [5-Cyano-8-methyl-1-propyl-7-(pyridin-4-ylmethoxy)-1,3,4,9-tetrahydro-pyrano[3,4-b]indol-1-yl]-acetic acid

To a solution of (R)-(5-cyano-7-hydroxy-8-methyl-1-propyl-1,3,4,9-tetrahydro-pyrano[3,4-b]indol-1-yl)-acetic acid ethyl ester (140 mg, 0.39 mmol) in THF (6 mL) was added 4-pyridylcarbinol (87 mg, 0.80 mmol) and triphenylphosphine (207 mg, 0.80 mmol). After cooling to 0° C., DIAD (157 μL, 0.80 mmol) was added. The cold bath was removed, and the solution was stirred at ambient temperature for 2.5 hours. The reaction was quenched with water and extracted with EtOAc. The organic layer washed with brine, dried (Na₂SO₄) and concentrated. The residue was dissolved in diethyl ether, and 4N HCl in dioxane (ca. 1 mL) was added. The precipitated solids were collected by filtration, washed with ether, and partitioned between EtOAc and 5% NaHCO₃. The aqueous layer was extracted with EtOAc. The combined organic layers were washed with H₂O and brine, dried (Na₂SO₄) and concentrated to afford 95.9 mg (54%) of a pale yellow solid.

To a solution of the ethyl ester (95 mg, 0.21 mmol) in EtOH (5 mL) was added 10% NaOH (aq., 0.5 mL). After stirring for 18 hours, H₂O was added and the pH brought to pH 6-7 with 3% HCl. The mixture was extracted with EtOAc (3×). The combined organic layers were washed with brine, dried (Na₂SO₄) and concentrated to afford 67.3 mg (76%) of the title compound as a pale yellow solid. ESI-MS m/z 420 (MH)+. ¹H NMR (d₆-DMSO) δ 10.97 (s, 1H), 8.60 (m, 2H), 7.47 (m, 2H), 7.28 (s, 1H), 5.25 (s, 2H), 3.93 (m, 2H), 2.94 (d, J=14.1 Hz, 1H), 2.83 (m, 2H), 2.72 (d, J=14.1 Hz, 1H), 2.47 (s, 3H), 1.99 (m, 2H), 1.20 (m, 2H), 0.80 (m, 3H).

### EXAMPLE 25

### (R)-[5-Cyano-7-(1,5-dimethyl-1H-pyrazol-3-ylmethoxy)-8-methyl-1-propyl-1,3,4,9-tetrahydro-pyrano[3,4-b]indol-1-yl]-acetic acid

### (±)-(5-Cyano-7-hydroxy-8-methyl-1-propyl-1,3,4,9-tetrahydro-pyrano[3,4-b]indol-1-yl)-acetic acid

To a solution of (±)-(5-cyano-7-hydroxy-8-methyl-1-propyl-1,3,4,9-tetrahydro-pyrano[3,4-b]indol-1-yl)-acetic acid ethyl ester (3.0 g, 8.43 mmol) in ethanol (120 mL) was added 2M sodium hydroxide (10 mL). After stirring for 20 hours, the reaction mixture was concentrated, and the resulting residue was taken up in water. The aqueous solution was acidified, and the solids collected by filtration and washed with water. The wet solids were dried overnight in a vacuum oven at 40° C. to afford 2.7 g (96% yield) of the product. ESI-MS m/z 329 (MH)+. ¹H NMR (d₆-DMSO) δ 11.96 (s, 1H), 10.71 (s, 1H), 9.35 (s, 1H), 6.93 (s, 1H), 3.95 (m, 2H), 2.93 (d, J=13.5 Hz, 1H), 2.80 (m, 2H), 2.71 (d, J=13.5 Hz, 1H), 2.33 (s, 3H), 1.96 (m, 2H), 1.28 (m, 1H), 0.80 (m, 4H).

### (R)-(5-Cyano-7-hydroxy-8-methyl-1-propyl-1,3,4,9-tetrahydro-pyrano[3,4-b]indol-1-yl)-acetic acid methyl ester

To a solution of (+/−)(5-cyano-7-hydroxy-8-methyl-1-propyl-1,3,4,9-tetrahydro-pyrano[3,4-b]indol-1-yl)-acetic acid (2.7 g, 8.22 mmol) in methanol (50 mL) at 60° C. was added a solution of (−) quinine (2.9 g, 8.94 mmol) in methanol (40 mL). After 30 minutes at 60° C., a white solid precipitated. The solution was allowed to stir for 20 hours at 60° C. After cooling to room temperature, the solids were recovered by filtration and washed with methanol to afford 2.3 g of the quinine salt of (R)-(5-cyano-7-hydroxy-8-methyl-1-propyl-1,3,4,9-tetrahydro-pyrano[3,4-b]indol-1-yl)-acetic acid. The solids were partitioned between 1M HCl (50 mL) and EtOAc (50 mL). The organic layer washed with brine, dried (MgSO₄), and concentrated to afford (R)-(5-cyano-7-hydroxy-8-methyl-1-propyl-1,3,4,9-tetrahydro-pyrano[3,4-b]indol-1-yl)-acetic acid as a white solid. The solids were taken up in toluene (30 mL) and trimethylorthoacetate (60 mL), and the solution was heated at 100° C. for 2 hours. The reaction was concentrated in vacuo, and the resulting residue was chromatographed on SiO₂ eluting with 30% EtOAc in hexanes to afford 1.25 g (46%) of the methyl ester whose chiral purity was determined to be 98.5% (97% ee, Chiral HPLC HP 100 with CHIRALPAK AS, 250×4.6 mm, 2-propanol/heptane (20/80), 1.0 mL/min, 235 nm UV detection; tR=6.35 min (R enantiomer), 8.53 min (S enantiomer)). ¹H NMR (CDCl₃) δ 9.40 (s,1H), 6.94 (s,1H), 4.69 (s,1H), 4.02 (m,1H), 3.92 (m,1H), 3.75 (s,3H), 2.94-3.06 (m, 2H), 2.41(s, 3H), 1.91-2.04 (m, 2H), 1.26-1.53 (m, 2H), 0.88 (t, J=7.0 Hz, 3H).

### [5-Cyano-7-(1,5-dimethyl-1H-pyrazol-3-ylmethoxy)-8-methyl-1-propyl-1,3,4,9-tetrahydro pyrano[3,4-b]indol-1-yl]-acetic acid

To a solution of 5-cyano-7-hydroxy-8-methyl-1-propyl-1,3,4,9-tetrahydro-pyrano[3,4-b]indol-1-yl)-acetic acid methyl ester (50 mg, 0.15 mmoles) in DCM (1 mL) was added (1,5-dimethyl-1H-pyrazol-3-yl)-methanol (20 mg, 0.17 mmoles) and triphenylphosphine (95 mg, 0.37 mmol). To this was added 1,1′-(azodicarbonyl)dipiperidine (ADDP, 92 mg, 0.37 mmoles). The reaction was stirred for 1.5 hours, diluted with EtOAc and washed twice with H₂O and brine. The EtOAc layer was dried (MgSO₄) and concentrated. Flash chromatography on SiO₂ eluting with 1:1 hexane/EtOAc yielded 53 mg (80%) of a yellow oil that crystallized upon standing.

To a solution of the methyl ester (53 mg, 0.12 mmol) in EtOH (5 mL) was added 2N NaOH (1 mL). The reaction was stirred at 40° C. for 1 hour, cooled, and concentrated in vacuo. The residue was taken up in H₂O, acidified, and extracted with EtOAc. The combined organic layers were washed with brine, dried (MgSO₄), and concentrated. Trituration of the solids with DCM/hexane afforded 36 mg (71%) of the acid as an off-white solid. ESI-ms m/z 437 (MH)+. ¹H NMR (d₆-DMSO) δ 11.98 (s, 1H), 10.87 (s, 1H), 7.32 (s, 1H), 6.10 (s, 1H), 4.98 (s, 2H), 3.95 (m, 2H), 3.69 (s, 3H), 2.93 (d, J=13.5 Hz, 1H), 2.82 (m, 2H), 2.70 (d, J=13.5 Hz, 1H), 2.35 (s, 3H), 2.23 (s, 3H), 1.96 (m, 2H), 1.16 (m, 1H), 0.82 (m, 4H).

### EXAMPLE 26

### (R)-[5-Cyano-7-(2-isopropoxy-ethoxy)-8-methyl-1-propyl-1,3,4,9-tetrahydro-pyrano[3,4-b]indol-1-yl]-acetic acid

### 2-(2-Bromoethoxy)propane

To a 2 neck flask equipped with a reflux condenser, magnetic stir bar and a septum was added 2-isopropoxyethanol (2 mL, 17.34 mmol). Phosphorous tribromide (0.52 mL, 5.45 mmol) was added dropwise via syringe. The reaction temperature increased and a slight reflux occurred. The reaction was stirred at room temperature under argon for 24 hours, quenched with water and extracted with hexane. The organic layer was dried over magnesium sulfate and concentrated to provide 1 g (34%) of the product as a colorless oil. ¹H NMR (CDCl₃) δ 3.78 (t, 2H); 3.61 (m,1H); 3.42 (t, 2H); 1.18 (d, 6H).

### (R)-[5-Cyano-7-(2-isopropoxy-ethoxy)-8-methyl-1-propyl-1,3,4,9-tetrahydro-pyrano[3,4-b]indol-1-yl]-acetic acid

To a solution of (R)-(5-cyano-7-hydroxy-8-methyl-1-propyl-1,3,4,9-tetrahydro-pyrano[3,4-b]indol-1-yl)-acetic acid methyl ester (125 mg, 0.365 mmol) and 2-(2-bromoethoxy)propane (0.07 mL, 0.539 mmol) in anhydrous N,N-dimethylformamide (2.5 mL) was added cesium carbonate (250 mg, 0.767 mmol) and a catalytic amount of sodium iodide. The reaction was heated at 60° C. for 1 hour, cooled to room temperature, quenched with water and extracted with ethyl acetate. The organic layer washed with brine, dried over magnesium sulfate and concentrated. Purification by column chromatography on silica gel using 3/1 hexane/ethyl acetate as eluent provided 52 mg (33%) of the product as a white solid. ESI-MS m/z 429 (M+H)⁺. ¹H NMR (CDCl₃) δ 9.36 (s, 1H); 7.07 (s, 1H); 4.14 (t, 2H); 4.05 (m, 1H); 3.90 (m, 1H); 3.78 (t, 2H); 3.74 (s, 3H); 3.68 (m 1H); 3.03 (m, 3H); 2.91 (d, 1H), 2.42 (s,3H); 2.03 (m 1H); 1.96 (m, 1H); 1.39 (m 1H), 1.22 (d, 6H); 1.17 (m,1H); 0.94 (t, 3H).

To a solution of the methyl ester (48 mg, 0.112 mmol) in ethanol (2 mL) was added 10% aqueous sodium hydroxide solution (0.2 mL, excess). After stirring at room temperature for 24 hours, the solvent was removed by rotary evaporation. The residue was dissolved in water and acidified with 1N HCl. The solid precipitate was collected by suction filtration, washed with water and dried to provide 43 mg (93%) of the product as a white solid. ESI-MS m/z 413.2 (M−H)⁻. ¹H NMR (d₆-DMSO) δ 11.98 (s, 1H); 10.90 (s, 1H); 7.20 (s, 1H); 4.11 (t, 2H); 3.89 (m, 2H); 3.69-3.58 (m, 3H); 2.90 (d, 1H), 2.81 (m, 2H); 2.68 (d, 1H); 2.37 (s, 3H); 1.96 (m, 2H); 1.28 (m, 1H); 1.10 (d, 6H); 0.76 (m, 4H).

### EXAMPLE 27

### (R)-[5-Cyano-7-(3-methoxy-propoxy)-8-methyl-1-propyl-1,3,4,9-tetrahydro-pyrano[3,4-b]indol-1-yl]-acetic acid

To a solution of (R)-(5-cyano-7-hydroxy-8-methyl-1-propyl-1,3,4,9-tetrahydro-pyrano[3,4-b]indol-1-yl)-acetic acid methyl ester (100 mg, 0.292 mmol) and 3-methoxypropyl bromide (67 mg, 0.438 mmol) in anhydrous N,N-dimethylformamide (2 mL) was added cesium carbonate (143 mg, 0.438 mmol) and a catalytic amount of sodium iodide. The reaction was heated at 60° C. for 24 hours, cooled to room temperature, quenched with water and extracted with ethyl acetate. The organic layer washed with brine, dried over magnesium sulfate and concentrated. Purification by column chromatography on silica gel using 2/1 hexane/ethyl acetate as eluent provided the product as a white solid. ESI-MS m/z 415 (M+H)⁺

To a solution of the methyl ester in ethanol (2 mL) was added 10% aqueous sodium hydroxide solution (0.2 mL, excess). After stirring at room temperature for 24 hours, the solvent was removed by rotary evaporation. The residue was dissolved in water and acidified with 1N HCl. The solid precipitate was collected by suction filtration, washed with water and dried to provide 33 mg (28% over two steps) of the product as a white solid. ESI-MS m/z 399 (M−H)⁻. ¹H NMR (DMSO) δ 11.99 (s, 1H); 10.87 (s, 1H); 7.17 (s, 1H); 4.05 (t, 2H); 3.91 (m, 2H); 3.49 (t, 2H); 3.24 (s, 3H); 2.89 (d, 1H); 2.81 (m, 2H); 2.72 (d, 1H); 2.36 (s, 3H); 1.94 (m, 4H); 0.77 (m, 4H).

### EXAMPLE 28

### (1R,2′R)-[5-Cyano-7-(2-methoxy-propoxy)-8-methyl-1-propyl-1,3,4,9-tetrahydro-pyrano[3,4-b]indol-1-yl]-acetic acid

### (R)-2-methoxypropan-1-ol

To a 2 neck flask fitted with a reflux condenser, an argon inlet and a septum was added lithium aluminum hydride (442 mg, 11.64 mmol) and diethyl ether (5 mL). The suspension was placed under argon, and a solution of ethyl(R)-(−)-2-methoxypropionate (2 g, 15.13 mmol) in diethyl ether (5 mL) was added dropwise via syringe. The mixture was heated at reflux for 1 hour, cooled to room temperature, and a solution of potassium hydroxide (1.7 g, 30.30 mmol) in water (4 mL) was added. The ether layer was decanted, and the aqueous mixture washed twice more with ether followed by decanting. The ether washes were combined, dried over magnesium sulfate and evaporated in a cold water bath under reduced pressure. Distillation of the mixture gave 818 mg (60%) of the product as a colorless oil, b.p=130° C. ¹H NMR (CDCl₃) δ 3.59 (m, 1H), 3.40 (s, 3H); 2.19 (br s, 1H); 1.39 (d, 2H); 1.11 (d, 3H).

### (1R,2′R)-[5-Cyano-7-(2-methoxy-propoxy)-8-methyl-1-propyl-1,3,4,9-tetrahydro-pyrano[3,4-b]indol-1-yl]-acetic acid

To a solution of (R)-(5-cyano-7-hydroxy-8-methyl-1-propyl-1,3,4,9-tetrahydro-pyrano[3,4-b]indol-1-yl)-acetic acid methyl ester (100 mg, 0.292 mmol), triphenylphosphine (115 mg, 0.438 mmol) and 1,1′-(azocarbonyl)dipiperidine (110 mg, 0.438 mmol in anhydrous dichloromethane (2 mL) was added a solution of 2-methoxypropan-1-ol (39 mg, 0.438 mmol) The reaction was stirred at room temperature under argon for 48 hours, and the solvent removed by rotary evaporation. Purification by column chromatography on silica gel using 2/1 hexane/ethyl acetate as eluent provided the product as a white solid. ESI-MS m/z 415 (M+H)⁺

To a solution of the methyl ester in ethanol (2 mL) was added 10% aqueous sodium hydroxide solution (0.2 mL, excess). After stirring at room temperature for 24 hours, the solvent was removed by rotary evaporation. The residue was dissolved in water and acidified with 1N HCl. The solid precipitate was collected by suction filtration, washed with water and dried to provide 27 mg (23% over two steps) of the product as a white solid. ESI-MS m/z 399 (M−H)⁻. ¹H NMR (d₆-DMSO) δ 11.99 (s, 1H), 10.88 (s, 1H); 7.21 (s, 1H); 3.99-3.88 (m, 4H); 3.67 (m,1H); 3.34 (s, 3H); 2.94 (d, 1H); 2.83 (m, 2H); 2.71 (d, 1H); 2.39 (s, 3H), 1.97 (m, 2H); 1.29 (m, 1H); 1.20 (d, 3H); 0.76 (m, 4H).

### EXAMPLE 29

### [5-Cyano-8-methyl-7-(5-methyl-[1,3,4]thiadiazol-2-ylmethoxy)-1-propyl-1,3,4,9-tetrahydro-pyrano[3,4-b]indol-1-yl]-acetic acid

### Chloro-acetic acid N¹-acetyl-hydrazide

In a 50-mL round bottom flask, 1.07 g (13.0 mmol) of acetic hydrazide was dissolved in water (5 mL) and combined with Na₂CO₃ (0.814 g, 7.69 mmol). The resulting mixture was first cooled in an ice-bath and then chloroacetyl chloride (1.1 mL, 13.8 mmol) was added dropwise over 10 minutes with stirring. After all of the chloroacetyl chloride had been added, the reaction was allowed to warm to room temperature and to continue to stir for 1.5 hours. The product was filtered out and dried in vacuo to afford 0.202 g (10%) of clean chloro-acetic acid N¹-acetyl-hydrazide: ¹H NMR (CDCl₃) δ 10.17 (s, 1H), 9.93 (s, 1H), 4.11 (s, 2H), 1.86 (s, 3H).

### 2-Chloromethyl-5-methyl-[1,2,4]thiadiazole

In a 250-mL round bottom flask, chloro-acetic acid N¹-acetyl-hydrazide (0.400 g, 2.66 mmol) was combined with THF (40 mL) and phosphorus pentasulfide (P₂S₅) (1.83 g, 4.12 mmol). The resulting mixture was heated at reflux for 2 hours and then cooled to room temperature. Once at room temperature, 5% Na₂CO₃ (16 mL) and excess diethyl ether were added; the resulting suspension was filtered. The organic layer was separated, dried over MgSO₄, filtered, and concentrated in vacuo to afford 0.160 g (41%) of 2-chloromethyl-5-methyl-[1,2,4]thiadiazole: ¹H NMR (CDCl₃) δ 4.91 (s, 2H), 2.80 (s, 3H).

### (R)-[5-Cyano-8-methyl-7-(5-methyl-[1,3,4]thiadiazol-2-ylmethoxy)-1-propyl-1,3,4,9-tetrahydro-pyrano{3,4-b]indol-1-yl]-acetic acid methyl ester

In a 50-mL round bottom flask and under argon, (R)-(5-cyano-7-hydroxy-8-methyl-1,3,4,9-tetrahydro-pyrano[3,4-b]indole-1-yl)-acetic acid methyl ester (0.100 g, 0.292 mmol) was combined with 2-chloromethyl-5-methyl-[1,2,4]thiadiazole (0.108 g, 0.730 mmol) and anhydrous NMP (4 mL). With stirring, K₂CO₃ (0.101 g, 0.730 mmol) and KI (0.012 g, 0.073 mmol) were added. The resulting mixture was allowed to continue stirring at room temperature for 24 hours. The reaction mixture was then poured onto water, and the product was extracted with EtOAc. The organic layer washed twice with ice-water, once with brine, dried over MgSO₄, filtered, and concentrated in vacuo to yield an oil that was then purified by flash chromatography to afford 0.084 g (63%) of (R)-[5-cyano-8-methyl-7-(5-methyl-[1,3,4]thiadiazol-2-ylmethoxy)-1-propyl-1,3,4,9-tetrahydro-pyrano[3,4-b]indol-1-yl]-acetic acid methyl ester: ¹H NMR (CDCl₃) δ 9.52 (s, 1H), 7.11 (s, 1H), 5.46 (s, 2H), 4.07 (m, 1H), 3.94 (m, 1H), 3.75 (s, 3H), 3.06 (m, 4H), 2.81 (s, 3H), 2.34 (s, 3H), 2.11 (m, 2H), 1.43 (m, 1H), 1.28 (m, 1H), 0.90 (t, J=7.3 Hz, 3H).

### (R)-[5-Cyano-8-methyl-7-(5-methyl-[1,3,4]thiadiazol-2-ylmethoxy)-1-propyl-1,3,4,9-tetrahydro-pyrano[3,4-b]indol-1-yl]-acetic acid

In a 25-mL round bottom flask, (R)-[5-cyano-8-methyl-7-(5-methyl-[1,3,4]thiadiazol-2-ylmethoxy)-1-propyl-1,3,4,9-tetrahydro-pyrano{3,4-b]indol-1-yl]-acetic acid methyl ester (0.084 g, 0.185 mmol) was combined with EtOH (8 mL) and 10% NaOH (0.38 mL). The resulting solution was allowed to stir for 24 hours at room temperature. The reaction solution was concentrated in vacuo to yield a residue that was then taken up in water and washed once with diethyl ether. The aqueous layer was then made acidic with 4M HCl, and the precipitated product was extracted with EtOAc, dried over MgSO₄, and concentrated in vacuo to afford 0.069 g (85%) of (R)-[5-cyano-8-methyl-7-(5-methyl-[1,3,4]thiadiazol-2-ylmethoxy)-1-propyl-1,3,4,9-tetrahydro-pyrano[3,4-b]indol-1-yl]-acetic acid: Purity 98% (HPLC); MS (M−H)⁻439.1 m/z; ¹H NMR (CDCl₃) δ 9.53 (s, 1H), 7.03 (s, 1H), 5.46 (d, J=2.9 Hz, 2H), 4.17 (m, 2H), 3.12 (m, 4H), 2.83 (s, 3H), 2.15 (s, 3H), 2.13 (m, 2H), 1.49 (m, 1H), 1.29 (m, 1H), 0.918 9 (t, J=7.3 Hz, 3H).

### EXAMPLE 30

### (R)-[5-Cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid

### 5-(N,N-Dimethylamino)-3-(chloromethyl)-1,2,4-thiadiazole

5-Chloro-3-(chloromethyl)-1,2,4-thiadiazole (0.2 g, 1.2 mmol) was dissolved in dry THF (2 mL) followed by the addition of a 2M solution of dimethylamine in THF (1.18 mL, 2.4 mmol). The reaction was stirred at room temperature overnight. The precipitated solid was filtered off, and the solvent removed under reduced pressure. The residue was taken up in ether, and washed with water and sat. NaCl. The organic layer was dried over MgSO₄, filtered, and the solvent was removed under reduced pressure to give an oil (0.210 g) in quantitative yield. ¹H NMR (CDCl₃) δ 4.51 (s, 2H), 3.16 (s, 6H).

### (R)-[5-Cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid methyl ester

(R)-(5-Cyano-7-hydroxy-8-methyl-1-propyl-1,3,4,9-tetrahydro-pyrano[3,4-b]indol-1-yl)-acetic acid methyl ester (0.1 g, 0.29 mmol) was dissolved in dry NMP (3 mL), followed by the addition of 5-(N,N-dimethylamino)-3-(chloromethyl)-1,2,4-thiadiazole (0.124 g, 0.70 mmol), milled potassium carbonate (0.096 g, 0.70 mmol) and potassium iodide (0.011 g, 0.070 mmol). The reaction was stirred at room temperature overnight. EtOAc (50 mL) was added to the reaction, and it washed several times with ice water and finally with sat. NaCl. The organic layer was dried over MgSO₄, filtered, and the solvent was removed under reduced pressure to give a residue that was purified by silica gel flash chromatography to give 0.104 g (74% yield) product as a white solid. Purity 99.3% (HPLC); MS (M−H)-482.2 m/z; ¹H NMR (CDCl₃) δ 9.38 (s, 1H), 7.18 (s, 1H), 5.11 (s, 2H), 4.05 (m, 1H), 3.90 (m, 1H), 3.75 (s, 3H), 3.18 (s, 6H), 3.03 (m, 3H), 2.92 (d, J=16.4 Hz, 1H), 2.48 (s, 3H), 2.06 (m, 1H), 1.92 (m, 1H), 1.38 (m, 1H), 1.19 (m, 1H), 0.89 (t, J=7.3 Hz, 3H).

### (R)-[5-Cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid

(R)-[5-Cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid methyl ester (0.100 g, 0.207 mmol) was dissolved in EtOH (4 mL), followed by the addition of a 10% sodium hydroxide solution (0.41 mL). The reaction was stirred overnight at room temperature, then the solvent was removed under reduced pressure. The residue was dissolved in water (50 mL) and washed with ether (2×25 mL). The aqueous layer was acidified with 1M HCl to about pH 4, then extracted with EtOAc. The organic layer was dried over MgSO₄, filtered, and solvent removed under reduced pressure to give the product (0.09 g, 93% yield) as an off white solid. Purity 97.5% (HPLC); MS (M+H)⁺ 470.1 m/z; ¹H NMR (CDCl₃) δ 11.98 (s, 1H), 10.91 (s, 1H), 7.31 (s, 1H), 5.09 (s, 2H), 3.92 (m, 2H), 3.10 (s, 6H), 2.93 (d, J=13.5 Hz, 1H), 2.82 (m, 2H), 2.71 (d, J=13.5 Hz, 1H), 2.41 (s, 3H), 1.97 (m, 2H), 1.30 (m, 1H), 0.78 (m, 4H).

### EXAMPLE 31

### 5-Cyano-7-(2-methoxy-ethoxy)-8-methyl-1-propyl-1,3,4,9-tetrahydro-pyrano[3,4,-b]indole-1-carboxylic acid

### 5-Bromo-2-methyl-3-nitrobenzoic acid

A 5000 mL three neck RBF equipped with an overhead stirrer was charged with 2-methyl-3-nitro benzoic acid (150.0 g, 0.82 mol) and concentrated H₂SO₄ (600 mL). To this solution was added 1,3-dibromo-5,5-dimethylhydantoin (130.7 g, 0.455 mol) over ten minutes with vigorous stirring. The reaction was vigorously stirred at ambient temperature for 5 hours. The reaction mixture was then added to H₂O (4000 mL) chilled in an ice bath over 30 minutes. This mixture was then filtered, the solids washed twice with H₂O and further dried under vacuum to yield 217.7 g (99.8%) of an off-white solid. ¹H NMR (CDCl₃) δ 8.18 (s, 1H), 7.95 (s, 1H), 2.59 (s, 3H).

### 5-Bromo-2-methyl-3-nitrobenzoic acid benzyl ester

A 5000 mL three neck RBF equipped with an overhead stirrer and thermometer was charged with 5-bromo-2-methyl-3-nitro benzoic acid (116.2 g, 0.45 mol), THF (1 L), and benzyl bromide (84.90 mL, 0.715 mol). To the stirring solution was added diisopropyl ethylamine (78 mL, 0.450 mol). The reaction was then brought to reflux. After 5.5 hours at reflux, the reaction was cooled to 40° C. and pyrrolidine (83 mL, 1.00 mol) was added. The reaction was stirred 10 minutes at 40° C., and then allowed to cool to ambient temperature over approximately 20 minutes. The reaction was diluted with EtOAc and washed 2×3% HCl, 1×H₂0, 1× saturated NaCl, dried (MgSO₄), and concentrated. The brown oil was taken up in DCM and slurried with approximately 300 g of silica gel. The slurry was filtered, solids washed with EtOAc, and the filtrate was concentrated to afford a yellow oil. Upon trituration in hexanes, 244.9 g (83.8%) of off white powder was obtained. ¹H NMR (CDCl₃) δ 8.10 (s, 1H), 7.96 (s, 1H), 7.41 (m, 5H), 5.37 (s, 2H), 2.55 (s, 3H).

### 3-Amino-5-bromo-2-methylbenzoic acid benzyl ester

A 1000 mL RBF equipped with an overhead stirrer was charged with of 5-bromo-2-methyl-3-nitro benzoic acid benzyl ester (75.0 g, 0.214 mol) and H₂O (200 mL). While stirring vigorously, Fe powder (˜325 mesh, 47.81 g, 0.857 mol) and NH₄Cl (13.88 g, 0.257 mol) were added. The mixture was heated to reflux for 4.5 hours. The reaction was then allowed to cool to room temperature, diluted with EtOAc, and filtered through a pad of Celite. The filtrate was washed with H₂O (2×) and saturated NaCl (1×), dried (MgSO₄), and concentrated. Upon trituration in hexanes, 63.2 g (92.1%) of off-white powder was obtained. ¹H NMR (CDCl₃) δ 7.38 (m, 6H), 6.90 (s, 1H), 5.31 (s, 2H), 3.76 (br s, 2H), 2.25 (s, 3H).

### 5-Bromo-3-hydrazino-2-methyl benzoic acid benzyl ester hydrochloride

A 1000 mL three neck RBF equipped with an overhead stirrer was charged with 3-amino-5-bromo-2-methyl benzoic acid benzyl ester (25.0 g, 0.078 mol), H₂O (150 mL), and concentrated HCl (150 mL). This mixture was stirred vigorously while cooling to −10° C. in a MeOH/ice bath. A solution of NaNO₂ (8.08 g, 0.120 mol) in H₂0 (150 mL) was cooled to −10° C. and then added to the reaction mixture dropwise over 15 minutes. The reaction was vigorously stirred at −10° C. for 1.5 hours, then a −10° C. solution of SnCl₂.2H₂0 (73.99 g, 0.330 mol) in concentrated HCl (150 mL) was added to the reaction mixture dropwise over 20 minutes. This was allowed to react for 1.5 hours at −10° C. with very vigorous stirring. The reaction mixture was then added to 6N NaOH (600 mL) and extracted with EtOAc. The organic layer was separated and washed with saturated NaCl, dried over MgSO₄, and concentrated. The yellow solid was taken up in THF (100 mL), diluted with 4N HCl dioxane (30 mL), and then the solvents were removed under vacuum. Trituration with DCM/hexanes yielded 27.1 g (93.5%) of white powder. ¹H NMR (d₆-DMSO) δ 10.47(br s, 2H), 8.39 (br s, 1H), 7.40 (m, 7H), 5.33 (s, 2H), 2.26 (s, 3H).

### 4-Bromo-3-(2-hydroxy-ethyl)-7-methyl-1H-indole-6-carboxylic acid benzyl ester

A 1000 mL RBF was charged with 5-bromo-3-hydrazino-2-methyl benzoic acid benzyl ester hydrochloride (30.0 g, 0.081 mol), ethylene glycol (350 mL), and H₂O (60 mL). This mixture was then heated to 40° C., and dihydrofuran (7.63 mL, 0.101 mol) was added. The reaction mixture was then heated to 100-105° C. with stirring for 2.25 hours. The reaction was cooled to ambient temperature, diluted with EtOAc, washed with saturated NaCl (2×), dried (MgSO4), and concentrated. The resulting red oil was then chromatographed on SiO₂ eluting with a 10% EtOAc/DCM to 50% EtOAc/DCM gradient. Isolation of the product as an impure solid followed by trituration with DCM and hexanes afforded 8.45 g (27.0%) of pale yellow solid. ¹H NMR (CDCl₃) δ 8.28 (br s, 1H), 7.94 (s, 1H), 7.40 (m, 6H), 5.36 (s, 1H), 3.96 (q, J=6.4 Hz, 2H), 3.28 (t, J=6.4 Hz, 2H), 2.72 (s, 3H),

### 5-Bromo-8-methyl-1-propyl-1,3,4,9-tetrahydro-pyrano[3,4,-b]indole-1,7-dicarboxylic acid 7-benzyl ester 1-ethyl ester

To a solution of 4-bromo-3-(2-hydroxy-ethyl)-7-methyl-1H-indole-6-carboxylic acid benzyl ester (0.96 g, 2.5 mmol) in dichloroethane (60 mL) was added 2-oxo-pentanoic acid ethyl ester (0.57 g, 3.9 mmol) in dichloroethane (5 mL). The solution was heated at 70° C. and BF₃—OEt₂ (0.80 mL, 6.3 mmol) was then added. The solution was cooled to room temperature and allowed to stir for 2 hours. The solution was diluted with brine, and extracted with EtOAc (3×), dried over Na₂SO₄, filtered, and concentrated. Purification of the residue by flash chromatography (SiO₂, 2/1 hexanes/EtOAc) gave pyranoindole (781 mg, 61%) as an orange oil. APCI (M+H) 461 m/z. ¹H NMR (CDCl₃) δ 8.60 (s, 1H), 7.89 (s, 1H), 7.46-7.32 (m, 5H), 5.35 (s, 2H), 4.34-4.21 (m, 4H), 4.08-3.91 (m, 1H), 3.18-3.12 (m, 2H), 2.70 (s, 3H), 2.10-2.00 (m, 2H), 1.37-1.32 (m, 4H), 0.95 (t, J=7.8 Hz, 3H).

### 5-Cyano-8-methyl-1-propyl-1,3,4,9-tetrahydro-pyrano[3,4,-b]indole-1,7-dicarboxylic acid 7-benzyl ester 1-ethyl ester

A solution of 5-bromo-8-methyl-1-propyl-1,3,4,9-tetrahydro-pyrano[3,4,-b]indole-1,7-dicarboxylic acid 7-benzyl ester 1-ethyl ester (861 mg, 1.7 mmol) in NMP (10 mL) was treated with CuCN (1.5 g, 17 mmol) at room temperature. The solution was then heated at 180° C. for 2 hours. The reaction was cooled to room temperature, diluted with H₂O and EtOAc, and filtered through Celite. The filter pad was thoroughly rinsed with EtOAc. The filtrate was extracted with brine (2×), dried over Na₂SO₄, filtered, and concentrated. Purification of the residual oil by flash chromatography (SiO₂, 3/1 hexanes/EtOAc) gave benzonitrile as a light yellow solid (680 mg, 87%). APCI (M+H) 461 m/z. ¹H NMR (CDCl₃) δ 8.69 (s, 1H), 8.12 (s, 1H), 7.48-7.36 (m, 5H), 5.37 (s, 2H), 4.33-4.26 (m, 3H), 3.99-3.91 (m, 1H), 3.13-3.02 (m, 2H), 2.83 (s, 3H), 2.20-2.14 (m, 1H), 2.12-2.02 (m, 1H), 1.43-1.33 (m, 1H), 1.34 (t, J=7.2 Hz, 3H), 1.31-1.22 (m, 1H), 0.90 (t, J=6.9 Hz, 3H).

### 5-Cyano-8-methyl-1-propyl-1,3,4,9-tetrahydro-pyrano[3,4,-b]indole-1,7-dicarboxylic acid 1-ethyl ester

A solution of 5-cyano-8-methyl-1-propyl-1,3,4,9-tetrahydro-pyrano[3,4,-b]indole-1,7-dicarboxylic acid 7-benzyl ester 1-ethyl ester (718 mg, 1.6 mmol) in a mixture of EtOAc/MeOH (30 mL/2/1) was treated with Pd(OH)₂ (280 mg) and subjected to H₂ using Parr shaker. After 16 hours, the solution was filtered through Celite. The filter cake was thoroughly rinsed with EtOAc and MeOH. The filtrate was concentrated to give the acid as a light yellow solid (551 mg, 93%). LCMS (M−H) 369 m/z. ¹H NMR (DMSO) δ 11.58 (s, 1H), 7.93 (s, 1H), 4.19-4.01 (m, 3H), 3.87-3.78 (m, 1H), 2.97-2.93 (m, 2H), 2.85 (s, 3H), 2.49-2.16 (m, 2H), 1.25-1.07 (m, 5H), 0.83 (t, J=7.5 Hz, 3H).

### 5-Cyano-7-hydroxymethyl-8-methyl-1-propyl-1,3,4,9-tetrahydro-pyrano[3,4,-b]indole-1-carboxylic acid ethyl ester

A solution of 5-cyano-8-methyl-1-propyl-1,3,4,9-tetrahydro-pyrano[3,4,-b]indole-1,7-dicarboxylic acid 1-ethyl ester (551 mg, 1.5 mmol) in THF (20 mL) was treated with BH₃-THF (1M, 4.5 mL) at 0° C. The solution was slowly warmed to room temperature. After 2 hours, the reaction mixture was diluted with 1M HCl and extracted with EtOAc (3×). The combined organics were washed with saturated NaHCO₃, dried over Na₂SO₄, filtered and concentrated. Purification of the residue by flash chromatography (SiO₂, 1/1 hexanes/EtOAc) provided 350 mg (66%) of the alcohol as a light yellow foam. GCMS M⁺ 356 m/z. ¹H NMR (CDCl₃) δ 8.77 (s, 1H), 7.77 (s, 1H), 4.73 (s, 2H), 4.34-4.24 (m, 3H), 3.99-3.90 (m, 1H), 3.17-3.03 (m, 2H), 2.38 (s, 3H), 2.35 (bs, 1H), 2.21-1.99 (m, 2H), 1.44-1.23 (m, 5H), 0.89 (t, J=7.8 Hz, 3H).

### 5-Cyano-7-formyl-8-methyl-1-propyl-1,3,4,9-tetrahydro-pyrano[3,4,-b]indole-1-carboxylic acid ethyl ester

A solution of 5-cyano-7-hydroxymethyl-8-methyl-1-propyl-1,3,4,9-tetrahydro-pyrano[3,4,-b]indole-1-carboxylic acid ethyl ester (350 mg, 0.98 mmol) in dichloroethane (20 mL) was treated with activated MnO₂ (0.58 g, 6.7 mmol) at room temperature. The solution was then heated at reflux for 1 hour. The mixture was filtered through a Celite/silica gel plug which was thoroughly rinsed with EtOAc. The filtrate was concentrated to give 341 mg (98%) of the aldehyde as a light yellow solid. GCMS M⁺ 354 m/z. ¹H NMR (CDCl₃) δ 10.28 (s, 1H), 8.92 (s, 1H), 7.92 (s, 1H), 4.35-4.24 (m, 3H), 3.98-3.92 (m, 1H), 3.16-3.09 (m, 2H), 2.87 (s, 3H), 2.39-2.00 (m, 2H), 1.45-1.37 (m, 4H), 0.91 (t, J=7.5 Hz, 3H).

### 5-Cyano-7-hydroxy-8-methyl-1-propyl-1,3,4,9-tetrahydro-pyrano[3,4,-b]indole-1-carboxylic acid ethyl ester

A solution of 5-cyano-7-formyl-8-methyl-1-propyl-1,3,4,9-tetrahydro-pyrano[3,4,-b]indole-1-carboxylic acid ethyl ester (146 mg, 0.36 mmol) in t-butanol (15 mL) was treated with SeO₂ (13 mg, 0.11 mmol) followed by H₂O₂ (30%, 0.2 mL) and TFA (2 drops). After 1.5 hours, additional SeO₂ (12 mg) and H₂O₂ (0.2 mL) were added. After 4 hours, the solution was diluted with buffer (pH 6.9) and extracted with EtOAc. The combined organics were dried over Na₂SO₄, filtered and concentrated. The crude material was used without further purification. LCMS (M−H) 423 m/z. The residue was diluted with MeOH (10 mL) and treated with 10% K₂CO₃ at room temperature. After 1 hour, the solution was concentrated. The material was extracted with EtOAc and 1M HCl (2×), dried over Na₂SO₄, filtered and concentrated to provide an off-white solid (47 mg, 33%-2 steps). LCMS (M−H) 341 m/z. ¹H NMR (CDCl₃) δ 8.20 (s, 1H), 6.97 (s, 1H), 5.10 (s, 1H), 4.30-4.23 (m, 3H), 3.99-3.91 (m, 1H), 3.13-2.99 (m, 2H), 2.40 (s, 3H), 2.17-1.93 (m, 2H), 1.42-1.29 (m, 5H), 0.89 (t, J=7.8 Hz, 3H).

### 5-Cyano-7-(2-methoxy-ethoxy)-8-methyl-1-propyl-1,3,4,9-tetrahydro-pyrano[3,4,-b]indole-1-carboxylic acid

A solution of 5-cyano-7-hydroxy-8-methyl-1-propyl-1,3,4,9-tetrahydro-pyrano[3,4,-b]indole-1-carboxylic acid ethyl ester (50 mg, 0.15 mmol) in DMF (5 mL) was added to a solution of bromoethyl-methylether (0.1 mL, 1.0 mmol) in DMF (2 mL) containing Cs₂CO₃ (20 mg, 61 mmol) at room temperature. The reaction was heated at 60° C. for 1 hour, cooled to room temperature, diluted with brine, and extracted with EtOAc (3×). The combined organics were dried over Na₂SO₄, filtered, and concentrated. Purification of the residue by flash chromatography (SiO₂, 2/1-1/1 hexanes/EtOAc) provided the ether as a yellow oil that was used without further purification. LCMS (M−H) 399.

A solution of the ethyl ester (30 mg, 0.07 mmol) in EtOH (5 mL) was treated with 1M NaOH (1 mL) at room temperature. After 16 hours, the solution was concentrated, diluted with H₂O, and extracted with EtOAc (2×). The combined aqueous layers were made acidic with 1M HCl, extracted with EtOAc, dried over Na₂SO₄, filtered and concentrated. The residue was purified by flash chromatography (SiO₂, 10% MeOH/DCM-3% TFA/10% MeOH/CHCl₃) followed by trituration with Et₂O/hexanes to give 7 mg (61%) of the formic acid as an off-white solid. LCMS (M−H) 371 m/z. ¹H NMR (CDCl₃+CD₃OD) δ 8.99 (s, 1H), 7.06 (s, 1H), 4.17-4.14 (m, 1H), 3.80-3.76 (m, 2H), 3.47 (s, 3H), 3.05 (m, 2H), 2.94 (m, 7H), 2.43 (s, 3H), 2.14-2.09 (m, 1H), 1.37-1.35 (m, 2H), 0.89 (t, J=7.2 Hz, 3H).

### EXAMPLE 32

### 5-Cyano-8-methyl-7-(5-methyl-isoxazol-3-ylmethoxy)-1-propyl-1,3,4,9-tetrahydro-pyrano[3,4,-b]indole-1-carboxylic acid

A solution of 5-cyano-7-hydroxy-8-methyl-1-propyl-1,3,4,9-tetrahydro-pyrano[3,4,-b]indole-1-carboxylic acid ethyl ester (25 mg, 0.07 mmol) in DMF (5 mL) was treated with Cs₂CO₃ (52 mg, 0.16 mmol) at room temperature. The solution was treated with a mixture of bromomethyl isoxazole (20 mg, 0.11 mmol) in DMF (2 mL) and then heated at 60° C. for 1.5 hours. The solution was cooled to room temperature and diluted with brine, extracted with EtOAc (3×), dried over Na₂SO₄, filtered and concentrated. Purification of the residue by flash chromatography (SiO₂, 2/1 hexanes/EtOAc) provided the ether as a yellow oil that was used without further purification. LCMS (M−H) 436 m/z.

A solution of the ethyl ester (36 mg, 0.08 mmol) in EtOH (10 mL) was treated with 1M NaOH (1 mL) at room temperature. The solution was heated at 60° C. for 0.5 hours and then concentrated. The residue was diluted with H₂O, acidified with 1M HCl, and extracted with EtOAc (3×). The combined organics were dried over Na₂SO₄, filtered, and concentrated. The material was purified by preparative LCMS which provided the formic acid as an off-white fluffy solid (12 mg, 36%) following lyophilization. LCMS (M−H) 408 m/z. ¹H NMR (CDCl₃) δ 8.38 (bs, 1H), 7.13 (s, 1H), 6.10 (s, 1H), 5.14 (s, 2H), 4.22-4.11 (m, 2H), 3.16-3.12 (m, 2H), 2.45 (s, 3H), 2.40 (s, 3H), 2.25-2.15 (m, 1H), 2.11-2.00 (m, 1H), 1.42-1.38 (m, 2H), 0.94 (t, J=7.8 Hz, 3H).

### EXAMPLE 33

### (1R*,10S)-[1-sec-Butyl-5-cyano-7-(2-ethoxy-ethoxy)-8-methyl-1,3,4,9-tetrahydro-pyrano[3,4-b]indol-1-yl]-acetic acid

### (1R*,10S)-5-Bromo-1-sec-butyl-1-ethoxycarbonylmethyl-8-methyl-1,3,4,9-tetrahydro-pyrano[3,4-b]indole-7-carboxylic acid benzyl ester

To a solution of 4-bromo-3-(2-hydroxy-ethyl)-7-methyl-1H-indole-6-carboxylic acid benzyl ester (2.00 g, 5.15 mmol) and (S)-ethyl 4-methyl-3-oxohexanate (0.89 g, 5.15 mmol) in CH₂Cl₂ (50 mL) was added BF₃.OEt₂ (0.80 g, 5.66 mmol) dropwise at room temperature. The solution was stirred for 4 hours then quenched by the addition of saturated aqueous Na₂CO₃ (10 mL). The reaction mixture was diluted with water and extracted three times with diethyl ether (250 mL). The combined organic layers were washed with brine, dried over anhydrous Na₂SO₄, filtered and concentrated to dryness. The crude product was purified by silica gel chromatography (5% to 20% EtOAc/hexane) to afford 0.77 g (28%) of the esters as an ˜1:1 mixture of 2 diastereomers. ¹H NMR (CDCl₃): ˜1:1 mixture of 2 diastereomers, δ 9.78 (br s, 0.5H, indole NH of diastereomer 1), 9.76 (br s, 0.5H, indole NH of diastereomer 2), 7.88 (s, 1H), 7.31-7.47 (m, 5H), 5.35 (s, 2H), 4.27-4.05 (m, 3H), 3.73 (m, 1H), 3.21-2.96 (m, 4.5H), 2.72 (s, 3H), 2.16 (m, 1H), 1.66 (m, 0.5H), 1.35 (m, 0.5H), 1.28 (t, J=7 Hz, 3H), 1.10 (m, 0.5H), 1.03 (d, J=7 Hz, 1.5H), 0.93 (t, J=7 Hz, 1.5H), 0.73 (t, J=7 Hz, 1.5H), 0.63 (d, J=7 Hz, 1.5H).

### (1R*,10S)-1-sec-Butyl-5-cyano-1-ethoxycarbonylmethyl-8-methyl-1,3,4,9-tetrahydro-pyrano[3,4-b]indole-7-carboxylic acid benzyl ester

A mixture of (1R*,10S)-5-bromo-1-sec-butyl-1-ethoxycarbonylmethyl-8-methyl-1,3,4,9-tetrahydro-pyrano[3,4-b]indole-7-carboxylic acid benzyl ester (0.77 g, 1.41 mmol) and CuCN (0.63 g, 7.09 mmol) in N-methyl-2-pyrrolidinone (30 mL) was immersed in a pre-heated oil bath (190° C.). After 2 hours, the reaction was complete by TLC analysis and cooled to ambient temperature. The reaction mixture was diluted with water (30 mL) and diethyl ether (30 mL) and filtered through a pad of Celite. The filtrate was extracted three times with diethyl ether (150 mL). The combined organic layers were washed with water (5×) and brine (1×), dried over anhydrous Na₂SO₄, filtered and concentrated to afford the crude ester as an ˜1:1 mixture of 2 diastereomers which was used directly in the next reaction. ¹H NMR (CDCl₃): ˜1:1 mixture of 2 diastereomers, δ 10.05 (br s, 0.5H, indole NH of diastereomer 1), 10.03 (br s, 0.5H, indole NH of diastereomer 2), 8.11 (s, 1H), 7.48-7.35 (m, 5H), 5.36 (s, 2H), 4.28-4.09 (m, 3H), 3.37 (m, 0.5H), 3.17-2.99 (m, 4H), 2.83 (s, 3H), 2.36 (m, 0.5H), 2.16 (m, 1H), 2.01 (m, 0.5H), 1.66 (m, 0.5H), 1.36 (m, 0.5H), 1.26 (t, J=7 Hz, 3H), 1.14 (m, 0.5H), 1.05 (d, J=6 Hz, 1.5H), 0.93 (t, J=8 Hz, 1.5H), 0.76 (t, J=7 Hz, 1.5H), 0.65 (d, J=6 Hz, 1.5H).

### (1R*,10S)-1-sec-Butyl-5-cyano-1-ethoxycarbonylmethyl-8-methyl-1,3,4,9-tetrahydro-pyrano[3,4-b]indole-7-carboxylic acid

A solution containing crude (1R*,10S)-1-sec-butyl-5-cyano-1-ethoxycarbonylmethyl-8-methyl-1,3,4,9-tetrahydro-pyrano[3,4-b]indole-7-carboxylic acid benzyl ester (0.81 g) and 10% Pd-on Carbon (0.2 g) in EtOAc (20 mL) was stirred under a balloon of hydrogen gas for 3 hours at ambient temperature. The reaction mixture was filtered through a pad of Celite®, washed with methanol and concentrated to afford the crude acid as an ˜1:1 mixture of 2 diastereomers which was used directly in the next reaction. ¹H NMR (CDCl₃): 1:1 mixture of 2 diastereomers, δ 10.12 (br s, 0.5H, indole NH of diastereomer 1), 10.10 (br s, 0.5H, indole NH of diastereomer 2), 8.20 (s, 1H), 4.30-4.08 (m, 2.5H), 3.78 (m, 1H), 3.40 (t, J=7 Hz, 1H), 3.19-2.98 (m, 3.5H), 2.88 (s, 3H), 2.43 (app t, J=7 Hz, 0.5H), 2.20 (m, 0.5H), 2.03 (m, 0.5H), 1.68 (m, 0.5H), 1.36 (m, 0.5H), 1.29 (t, J=7 Hz, 3H), 1.14 (m, 0.5H), 1.06 (d, J=6 Hz, 1.5H), 0.94 (t, J=8 Hz, 1.5H), 0.75 (t, J=7 Hz, 1.5H), 0.65 (d, J=6 Hz, 1.5H).

### (1R*,10S)-(1-sec-Butyl-5-cyano-7-hydroxymethyl-8-methyl-1,3,4,9-tetrahydro-pyrano[3,4-b]indol-1-yl)-acetic acid ethyl ester

To a solution of crude (1R*,10S)-1-sec-butyl-5-cyano-1-ethoxycarbonylmethyl-8-methyl-1,3,4,9-tetrahydro-pyrano[3,4-b]indole-7-carboxylic acid (0.6 g) in THF (20 mL) at 0° C. was added BH₃.THF (1.0M in THF, 3 mL, 3.00 mmol). The cooling bath was removed, and the solution was stirred for 1 hour. The reaction mixture was re-cooled to 0° C. and a second portion of BH₃.THF (1.0M in THF, 3 mL, 3.00 mmol) was added. The reaction mixture was stirred an additional 1 hour at ambient temperature then carefully quenched with water. The reaction mixture was diluted with 1.0 N HCl and EtOAc, and the layers were separated. The organic layer washed with water and brine, dried over anhydrous Na₂SO₄, filtered and concentrated to afford the crude alcohol as an ˜1:1 mixture of 2 diastereomers which was used directly in the next reaction. ¹H NMR (CDCl₃): ˜1:1 mixture of 2 diastereomers, δ 9.66 (br s, 1H, indole NH), 7.43 (s, 1H), 4.80 (s, 2H), 4.27-4.06 (m, 3H), 3.77 (m, 1H), 3.16-2.98 (m, 3H), 2.57 (s, 3H), 2.19 (m, 0.5H), 1.63 (m, 0.5H), 1.34 (m, 0.5H), 1.28 (t, J=7 Hz, 3H), 1.18 (m, 0.5H), 1.04 (d, J=6 Hz, 1.5H), 0.93 (t, J=8 Hz, 1.5H), 0.74 (t, J=7 Hz, 1.5H), 0.63 (d, J=6 Hz, 1.5H).

### (1R*,10S)-(1-sec-Butyl-5-cyano-7-formyl-8-methyl-1,3,4,9-tetrahydro-pyrano[3,4-b]indol-1-yl)-acetic acid ethyl ester

To a solution of crude (1R*,10S)-(1-sec-butyl-5-cyano-7-hydroxymethyl-8-methyl-1,3,4,9-tetrahydro-pyrano[3,4-b]indol-1-yl)-acetic acid ethyl ester (0.57 g) in anhydrous benzene (20 mL) was added a homogeneous mixture of activated 85% MnO₂ (<5μ particle size, 0.3 g, 2.96 mmol) and Celite® (0.5 g). The mixture was immersed in a pre-heated oil bath (60° C.) and agitated vigorously. After 1 hour, a second portion of MnO₂ (1.0 g) and Celite® (1.0 g) was added and heating was continued for 1 hour. The reaction mixture was cooled to ambient temperature then filtered through Celite®. The solids were washed with EtOAc, and the yellow filtrate was concentrated to afford the crude aldehyde as an ˜1:1 mixture of 2 diastereomers which was used directly in the next reaction. ¹H NMR (CDCl₃): ˜1:1 mixture of 2 diastereomers, δ 10.27 (s, 1H), 7.90 (s, 1H), 4.30-4.11 (m, 3H), 3.79 (m, 1H), 3.18-3.02 (m, 3H), 2.88 (s, 3H), 2.17 (m, 0.5H), 1.67 (m, 0.5H), 1.36 (m, 0.5H), 1.30 (t, J=7 Hz, 3H), 1.23 (m, 0.5H), 1.06 (d, J=6 Hz, 1.5H), 0.94 (t, J=8 Hz, 1.5H), 0.75 (t, J=7 Hz, 1.5H), 0.64 (d, J=6 Hz, 1.5H).

### (1R*,10S)-(1-see-Butyl-5-cyano-7-hydroxy-8-methyl-1,3,4,9-tetrahydro-pyrano[3,4-b]indol-1-yl)-acetic acid ethyl ester

To a solution of (1R*,10S)-(1-sec-butyl-5-cyano-7-formyl-8-methyl-1,3,4,9-tetrahydro-pyrano[3,4-b]indol-1-yl)-acetic acid ethyl ester (0.53 g) in tert-butanol (10 mL) was added SeO₂ (46 mg, 0.41 mmol) followed by 30% H₂O₂ (5 mL) and trifluoroacetic acid (20 μL). The reaction mixture was stirred at ambient temperature for 1 hour, and then diluted with EtOAc, washed with saturated NaHCO₃ (2×), water and brine; dried over anhydrous Na₂SO₄; filtered and concentrated to afford the crude formate ester as an ˜1:1 mixture of 2 diastereomers which was used directly in the next reaction.

The crude residue was dissolved in MeOH (15 mL) and to this was added 10% aqueous K₂CO₃ (10 mL). After 1 hour, the reaction mixture was diluted with EtOAc, washed with water (2×) and brine, dried over anhydrous Na₂SO₄, filtered and concentrated. The crude phenol was purified by silica gel chromatography (20% to 30% EtOAc/hexane) to afford 0.19 g of (1R*,10S)-(1-sec-butyl-5-cyano-7-hydroxy-8-methyl-1,3,4,9-tetrahydro-pyrano[3,4-b]indol-1-yl)-acetic acid ethyl ester (36% over 6 steps) as an ˜1:1 mixture of diastereomers. ¹H NMR (CDCl₃): ˜1:1 mixture of 2 diastereomers, δ 9.38 (br s, 0.5H, indole NH of diastereomer 1), 9.36 (br s, 0.5H, indole NH of diastereomer 2), 6.96 (s, 1H), 5.26 (br s, 1H), 4.26-4.06 (m, 3H), 3.76 (m, 1H), 3.15-2.96 (m, 3H), 2.41 (s, 3H), 2.16 (m, 0.5H), 1.68 (m, 0.5H), 1.35 (m, 0.5H), 1.27 (t, J=7 Hz, 3H), 1.14 (m, 0.5H), 1.03 (d, J=6 Hz, 1.5H), 0.93 (t, J=8 Hz, 1.5H), 0.75 (t, J=7 Hz, 1.5H), 0.65 (d, J=6 Hz, 1.5H).

### (1R*,10S)-[1-sec-Butyl-5-cyano-7-(2-ethoxy-ethoxy)-8-methyl-1,3,4,9-tetrahydro-pyrano[3,4-b]indol-1-yl]-acetic acid ethyl ester

To a solution containing (1R*,10S)-(1-sec-butyl-5-cyano-7-hydroxy-8-methyl-1,3,4,9-tetrahydro-pyrano[3,4-b]indol-1-yl)-acetic acid ethyl ester (50 mg, 0.13 mmol) in anhydrous DMF (5 mL) was added Cs₂CO₃ (66 mg, 0.20 mmol) and 1-bromo-2-ethoxyethane (23 μL, 0.20 mmol). The reaction mixture was immersed in a pre-heated oil bath (60° C.). After 2 hours, additional 1-bromo-2-ethoxyethane (23 μL, 0.20 mmol) was added, and the reaction mixture was maintained at 60° C. for 3 hours. The reaction mixture was cooled to ambient temperature and diluted with diethyl ether and water. The layers were separated, and the organic phase was washed with water (5×) and brine (1×), dried over anhydrous Na₂SO₄, filtered and concentrated to afford the crude product as an ˜1:1 mixture of 2 diastereomers which was used directly in the next reaction. ¹H NMR (CDCl₃): ˜1:1 mixture of 2 diastereomers, δ 9.40 (br s, 0.5H, indole NH of diastereomer 1), 9.38 (br s, 0.5H, indole NH of diastereomer 2), 7.06 (s, 1H), 4.26-4.01 (m, 5H), 3.82-3.72 (m, 3H), 3.62 (t, J=7 Hz, 2H), 3.15-2.97 (m, 3H), 2.42 (s, 3H), 2.17 (m, 0.5H), 1.66 (m, 0.5H), 1.35 (m, 0.5H), 1.32-1.18 (m, 6H), 1.14 (m, 0.5H), 1.03 (d, J=6 Hz, 1.5H), 0.93 (t, J=8 Hz, 1.5H), 0.75 (t, J=7 Hz, 1.5H), 0.65 (d, J=6 Hz, 1.5H) ppm.

### (1R*,10S)-[1-sec-Butyl-5-cyano-7-(2-ethoxy-ethoxy)-8-methyl-1,3,4,9-tetrahydro-pyrano[3,4-b]indol-1-yl]-acetic acid

To a solution containing crude (1R*,10S)-[1-sec-butyl-5-cyano-7-(2-ethoxy-ethoxy)-8-methyl-1,3,4,9-tetrahydro-pyrano[3,4-b]indol-1-yl]-acetic acid ethyl ester (˜50 mg) in ethanol (4 mL) and THF (1 mL) was added 3 N NaOH (1 mL) at ambient temperature. After 5 hours, the reaction mixture was diluted with water and washed with diethyl ether. The aqueous phase was acidified with 1 N HCl, and the product was extracted with diethyl ether. The ether extract was washed with 1 N HCl and brine, dried over anhydrous Na₂SO₄, filtered and concentrated. The crude product was purified by silica gel chromatography (1% to 20% MeOH/DCM) and the purified acid was triturated with diethyl ether/hexane. The product was collected on a Hirsch funnel, washed with hexane then dried in vacuo to afford 34 mg of (1R*,10S)-[1-sec-butyl-5-cyano-7-(2-ethoxy-ethoxy)-8-methyl-1,3,4,9-tetrahydro-pyrano[3,4-b]indol-1-yl]-acetic acid (62% over 2 steps) as an ˜1:1 mixture of 2 diastereomers. ESI MS m/z 413.2 (M−H)⁻. ¹H NMR (CDCl₃): ˜1:1 mixture of 2 diastereomers, δ 9.13 (br s, 0.5H, indole NH of diastereomer 1), 9.09 (br s, 0.5H, indole NH of diastereomer 2), 7.05 (s, 1H), 4.18-4.00 (m, 2H), 3.97-3.77 (m, 3H), 3.63 (t, J=7 Hz, 2H), 3.50 (t, J=7 Hz, 2H), 3.13-3.03 (m, 4H), 2.27 (s, 3H), 2.17-1.61 (m, 2H), 1.36-1.06 (m, 5H), 1.04 (d, J=6 Hz, 1.5H), 0.93 (t, J=8 Hz, 1.5H), 0.75 (t, J=7 Hz, 1.5H), 0.65 (d, J=6 Hz, 1.5H).

### EXAMPLE 34

### (1R*,10S) [1-sec-Butyl-5-cyano-7-(2-isopropoxy-ethoxy)-8-methyl-1,3,4,9-tetrahydro-pyrano[3,4-b]indol-1-yl]-acetic acid

### (1R*,10S)-[1-see-Butyl-5-cyano-7-(2-isopropoxy-ethoxy)-8-methyl-1,3,4,9-tetrahydro-pyrano[3,4-b]indol-1-yl]-acetic acid ethyl ester

To a solution containing (1R*,10S)-(1-sec-butyl-5-cyano-7-hydroxy-8-methyl-1,3,4,9-tetrahydro-pyrano[3,4-b]indol-1-yl)-acetic acid ethyl ester (50 mg, 0.13 mmol) in anhydrous DMF (5 mL) was added Cs₂CO₃ (66 mg, 0.20 mmol) and 1-bromo-2-isopropoxyethane (˜40 μL, >0.20 mmol). The reaction mixture was immersed in a pre-heated oil bath (60° C.). After 4 hours, additional 1-bromo-2-isopropoxyethane (˜40 μL, >0.20 mmol) was added, and the reaction mixture was maintained at 60° C. for 3 hours. The reaction mixture was cooled to ambient temperature and diluted with diethyl ether and water. The layers were separated, and the organic phase washed with water (5×) and brine (1×), dried over anhydrous Na₂SO₄, filtered and concentrated. The crude ester was purified by silica gel chromatography (25% EtOAC/hexane) to afford the ester as an ˜1:1 mixture of 2 diastereomers. ¹H NMR (CDCl₃): ˜1:1 mixture of 2 diastereomers, δ 9.40 (br s, 0.5H, indole NH of diastereomer 1), 9.38 (br s, 0.5H, indole NH of diastereomer 2), 7.06 (s, 1H), 4.35-3.92 (m, 5H), 3.79-3.57 (m, 3H), 3.44 (m, 2H), 3.19-2.80 (m, 3H), 2.40 (s, 3H), 2.17 (m, 0.5H), 1.66 (m, 0.5H), 1.39-1.09 (m, 12H), 1.03 (d, J=6 Hz, 1.5H), 0.93 (t, J=8 Hz, 1.5H), 0.75 (m, 3H).

### (1R*,10S)-[1-sec-Butyl-5-cyano-7-(2-isopropoxy-ethoxy)-8-methyl-1,3,4,9-tetrahydro-pyrano[3,4-b]indol-1-yl]-acetic acid

To a solution containing crude (1R*,10S)-[1-sec-butyl-5-cyano-7-(2-isopropoxy-ethoxy)-8-methyl-1,3,4,9-tetrahydro-pyrano[3,4-b]indol-1-yl]-acetic acid ethyl ester (˜50 mg) in ethanol (4 mL) and THF (1 mL) was added 3 N NaOH (1 mL) at ambient temperature. After 5 hours, the reaction mixture was diluted with water and washed with diethyl ether. The aqueous phase was acidified with 1 N HCl, and the product was extracted with diethyl ether. The ether extract washed with 1 N HCl and brine, dried over anhydrous Na₂SO₄, filtered and concentrated. The crude product was purified by silica gel chromatography (1% to 20% MeOH/DCM), and the purified acid was triturated with diethyl ether/hexane. The product was collected on a Hirsch funnel, washed with hexane, and dried in vacuo to afford 16 mg of (1R*,10S)-[1-sec-butyl-5-cyano-7-(2-isopropoxy-ethoxy)-8-methyl-1,3,4,9-tetrahydro-pyrano[3,4-b]indol-1-yl]-acetic acid (29% over 2 steps) as an ˜1:1 mixture of 2 diastereomers. ESI MS m/z 429.2 (M+H)⁺. ¹H NMR (CDCl₃): ˜1:1 mixture of 2 diastereomers, δ 8.88 (br s, 0.5H, indole NH of diastereomer 1), 8.83 (br s, 0.5H, indole NH of diastereomer 2), 7.06 (s, 1H), 4.19-4.11 (m, 3H), 3.89 (m, 1H), 3.78 (app t, J=5 Hz, 2H), 3.69 (m, 1H), 3.12-3.04 (m, 4H), 2.32 (s, 3H), 2.17 (m, 1H), 1.65 (m, 1H), 1.27 (m, 1H), 1.21 (d, J=5 Hz, 6H), 1.05 (d, J=7 Hz, 1.5H), 0.93 (t, J=8 Hz, 1.5H), 0.77 (m, 3H).

### EXAMPLE 35

### (1R*,10S)-[1-sec-Butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid

### (1R*,10S)-[1-sec-Butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid ethyl ester

To a solution containing (1R*,10S)-(1-sec-butyl-5-cyano-7-hydroxy-8-methyl-1,3,4,9-tetrahydro-pyrano[3,4-b]indol-1-yl)-acetic acid ethyl ester (43 mg, 0.11 mmol) in anhydrous NMP (2 mL) was added Cs₂CO₃ (57 mg, 0.17 mmol), KI (19 mg, cat.), and 3-chloromethyl-5-dimethylamino-1,2,4-thiadiazole (27 mg, 0.17 mmol). The reaction mixture was maintained at ambient temperature. After 16 hours, the reaction mixture was diluted with diethyl ether and 10% aqueous HCl. The layers were separated, and the organic phase washed with water (5×) and brine (1×), dried over anhydrous Na₂SO₄, filtered and concentrated. The crude ester was purified by silica gel chromatography (30% to 60% EtOAC/hexane) to afford 46 mg of the ester as an ˜1:1 mixture of 2 diastereomers. ¹H NMR (CDCl₃): ˜1:1 mixture of 2 diastereomers, δ 9.42 (br s, 0.5H, indole NH of diastereomer 1), 9.39 (br s, 0.5H, indole NH of diastereomer 2), 7.18 (s, 1H), 5.11 (s, 2H), 4.30-4.04 (m, 3H), 3.75 (m, 1H), 3.39 (t, J=7 Hz, 1H), 3.18 (s, 6H), 3.13-2.95 (m, 3H), 2.44 (s, 3H), 2.39 (m, 1H), 2.18 (m, 1H), 2.01 (m, 1H), 1.66 (m, 0.5H), 1.30 (t, J=7 Hz, 3H), 1.35 (m, 0.5H), 1.16 (m, 0.5H), 1.03 (d, J=6 Hz, 1.5H), 0.93 (t, J=8 Hz, 1.5H), 0.75 (t, J=7 Hz, 1.5H), 0.63 (d, J=7 Hz, 1.5H).

### (1R*,10S)-[1-sec-Butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid

To a solution containing (1R*,10S)-[1-sec-butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid ethyl ester (46 mg) in ethanol (2 mL) and THF (1 mL) was added 3 N NaOH (1 mL) at ambient temperature. After 16 hours, the reaction mixture was diluted with water and acidified with 1 N HCl. The product was extracted with diethyl ether. The ether extract washed with 1 N HCl and brine, dried over anhydrous Na₂SO₄, filtered and concentrated. The crude product was triturated with diethyl ether/hexane. The product was collected on a Hirsch funnel, washed with hexane, and dried in vacuo to afford 29 mg of (1R*,10S)-[1-sec-butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid (54% over 2 steps) as an ˜1:1 mixture of 2 diastereomers. ESI MS 71/z 484.1 (M+H)⁺. ¹H NMR (CDCl₃): ˜1:1 mixture of 2 diastereomers, δ 9.04 (br s, 0.5H, indole NH of diastereomer 1), 8.98 (br s, 0.5H, indole NH of diastereomer 2), 7.15 (s, 1H), 5.09 (s, 2H), 4.12 (m, 1H), 3.88 (m, 1H), 3.19 (s, 6H), 3.13-2.98 (m, 3H), 2.38 (s, 3H), 2.20 (m, 1H), 1.68 (m, 1H), 1.28 (m, 1H), 1.12 (m, 1H), 1.09 (d, J=6 Hz, 1.5H), 0.93 (t, J=8 Hz, 1.5H), 0.75 (t, J=7 Hz, 1.5H), 0.63 (d, J=7 Hz, 1.5H).

### EXAMPLE 36

### (1R*,10S)-[1-sec-Butyl-5-cyano-7-(1,5-dimethyl-1H-pyrazol-3-ylmethoxy)-8-methyl-1,3,4,9-tetrahydro-pyrano[3,4-b]indol-1-yl]-acetic acid

### (1R*,10S)-[1-sec-Butyl-5-cyano-7-(1,5-dimethyl-1H-pyrazol-3-ylmethoxy)-8-methyl-1,3,4,9-tetrahydro-pyrano[3,4-b]indol-1-yl]-acetic acid ethyl ester

To a solution containing (1R*,10S)-(1-sec-butyl-5-cyano-7-hydroxy-8-methyl-1,3,4,9-tetrahydro-pyrano[3,4-b]indol-1-yl)-acetic acid ethyl ester (47 mg, 0.12 mmol) in anhydrous NMP (2 mL) was added Cs₂CO₃ (83 mg, 0.25 mmol) and 3-bromomethyl-1,5-dimethylpyrrazole (48 mg, 0.25 mmol). The reaction mixture was maintained at ambient temperature. After 16 hours, the reaction mixture was diluted with diethyl ether and 10% aqueous HCl. The layers were separated, and the organic phase washed with water (5×) and brine (1×), dried over anhydrous Na₂SO₄, filtered and concentrated. The crude ester was purified by silica gel chromatography (50% to 60% EtOAC/hexane) to afford the ester as an ˜1:1 mixture of 2 diastereomers. ¹H NMR (CDCl₃): ˜1:1 mixture of 2 diastereomers, δ 9.37 (br s, 0.5H, indole NH of diastereomer 1), 9.35 (br s, 0.5H, indole NH of diastereomer 2), 7.16 (s, 1H), 6.10 (s, 1H), 5.02 (s, 2H), 4.26-4.06 (m, 3H), 3.77 (s, 3H), 3.74 (m, 2H), 3.15-2.96 (m, 3H), 2.41 (s, 3H), 2.27 (s, 3H), 2.17 (m, 1H), 1.66 (m, 0.5H), 1.35 (m, 0.5H), 1.26 (t, J=7 Hz, 3H), 1.16 (m, 0.5H), 1.03 (d, J=6 Hz, 1.5H), 0.94 (t, J=8 Hz, 1.5H), 0.76 (t, J=7 Hz, 1.5H), 0.65 (d, J=7 Hz, 1.5H).

### (1R*,10S)-[1-sec-Butyl-5-cyano-7-(1,5-dimethyl-1H-pyrazol-3-ylmethoxy)-8-methyl-1,3,4,9-tetrahydro-pyrano[3,4-b]indol-1-yl]-acetic acid

To a solution containing (1R*,10S)-[1-sec-butyl-5-cyano-7-(1,5-dimethyl-1H-pyrazol-3-ylmethoxy)-8-methyl-1,3,4,9-tetrahydro-pyrano[3,4-b]indol-1-yl]-acetic acid ethyl ester (˜50 mg) in ethanol (4 mL) and THF (1 mL) was added 3 N NaOH (2 mL) at ambient temperature. After 16 hours, the reaction mixture was diluted with water and acidified with 1 N HCl. The product was extracted with diethyl ether. The ether extract washed with 1 N HCl and brine, dried over anhydrous Na₂SO₄, filtered and concentrated. The crude product was triturated with diethyl ether/hexane. The product was collected on a Hirsch funnel, washed with hexane then dried in vacuo to afford 27 mg of (1R*,10S)-[1-sec-butyl-5-cyano-7-(1,5-dimethyl-1H-pyrazol-3-ylmethoxy)-8-methyl-1,3,4,9-tetrahydro-pyrano[3,4-b]indol-1-yl]-acetic acid (50% over 2 steps) as an ˜1:1 mixture of 2 diastereomers. ESI MS m/z 449.2 (M−H)⁻. ¹H NMR (CDCl₃): ˜1:1 mixture of 2 diastereomers, δ 9.16 (br s, 0.5H, indole NH of diastereomer 1), 9.13 (br s, 0.5H, indole NH of diastereomer 2), 7.06 (s, 1H), 6.12 (s, 1H), 4.99 (m, 2H), 4.13-4.08 (m, 1H), 3.85 (m, 1H), 3.78 (s, 3H), 3.12 (m, 2H), 3.00 (m, 3H), 2.28 (s, 3H), 2.23 (s, 3H), 1.70 (m, 0.5H), 1.35 (m, 0.5H), 1.16 (m, 0.5H), 1.06 (d, J=6 Hz, 1.5H), 0.94 (t, J=8 Hz, 1.5H), 0.75 (t, J=7 Hz, 1.5H), 0.68 (d, J=7 Hz, 1.5H).

By appropriate selection of suitable starting materials, other compounds of the invention may be prepared according to the procedures described in the foregoing examples. Representative examples of further pyranoindole derivatives and analogues thus prepared are set forth in Table 2 below.

TABLE 2
Pyranoindole derivatives

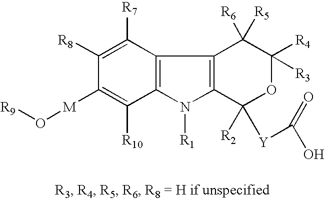

| | | | | | | | | Mass Spec* |
|---|---|---|---|---|---|---|---|---|
| Example | R₁ | R₂ | R₇ | R₉ | R₁₀ | Y | M | (m/z) |
| 1 | H | n-Propyl | CN | H | CH₃ | CH₂ | CH₂ | 341 (M − H)⁻ |
| 2 | H | n-Propyl | CN | CH₃ | CH₃ | CH₂ | CH₂ | 355 (M − H)⁻ |
| 3 | H | n-Propyl | CN | CH₂CH₃ | CH₃ | CH₂ | CH₂ | 369 (M − H)⁻ |
| 4 | H | n-Propyl | CN | 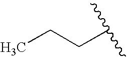 | CH₃ | CH₂ | CH₂ | 383 (M − H)⁻ |
| 5 | H | n-Propyl | CN | 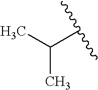 | CH₃ | CH₂ | CH₂ | — |
| 6 | H | n-Propyl | CN | 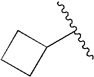 | CH₃ | CH₂ | CH₂ | 395 (M − H)⁻ |
| 7 | H | n-Propyl | CN | 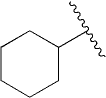 | CH₃ | CH₂ | CH₂ | 423 (M − H)⁻ |
| 8 | H | n-Propyl | CN | 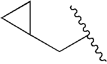 | CH₃ | CH₂ | CH₂ | 395 (M − H)⁻ |
| 9 | H | n-Propyl | CN | 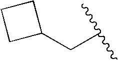 | CH₃ | CH₂ | CH₂ | 409 (M − H)⁻ |
| 10 | H | n-Propyl | CN | 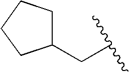 | CH₃ | CH₂ | CH₂ | 423 (M − H)⁻ |
| 11 | H | n-Propyl | CN | 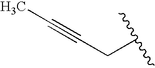 | CH₃ | CH₂ | CH₂ | 393 (M − H)⁻ |
| 12 | H | n-Propyl | CN | 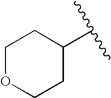 | CH₃ | CH₂ | CH₂ | 425 (M − H)⁻ |
| 13 | H | n-Propyl | CN | 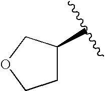 | CH₃ | CH₂ | CH₂ | 411 (M − H)⁻ |
| 14 | H | n-Propyl | CN | 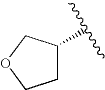 | CH₃ | CH₂ | CH₂ | 411 (M − H)⁻ |
| 15 | H | n-Propyl | CN | 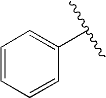 | CH₃ | CH₂ | CH₂ | 417 (M − H)⁻ |
| 16 | H | n-Propyl | CN | 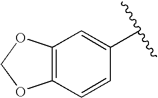 | CH₃ | CH₂ | CH₂ | 475 (M − H)⁻ |
| 17 | H | n-Propyl | CN | 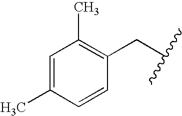 | CH₃ | CH₂ | CH₂ | 459 (M − H)⁻ |
| 18 | H | n-Propyl | CN | 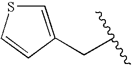 | CH₃ | CH₂ | CH₂ | 437 (M − H)⁻ |
| 19 | H | n-Propyl | CN | 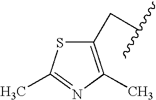 | CH₃ | CH₂ | CH₂ | 466 (M − H)⁻ |
| 20 | H | n-Propyl | CN | 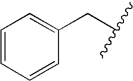 | CH₃ | CH₂ | CH₂ | 431 (M − H)⁻ |
| 21 | H | n-Propyl | CN | 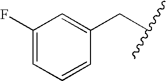 | CH₃ | CH₂ | CH₂ | 435 (M − H)⁻ |
| 22 | H | n-Propyl | CN | 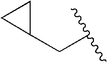 | CH₃ | CH₂ | bond | 383 (M + H)⁺ |
| 23 | H | n-Propyl (1R) | CN | 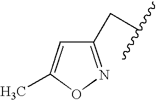 | CH₃ | CH₂ | bond | 424 (M + H)⁺ |
| 24 | H | n-Propyl (1R) | CN | 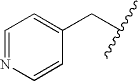 | CH₃ | CH₂ | bond | — |
| 25 | H | n-Propyl (1R) | CN | 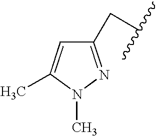 | CH₃ | CH₂ | bond | 435 (M − H)⁻ |
| 26 | H | n-Propyl (1R) | CN | 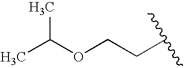 | CH₃ | CH₂ | bond | 413 (M − H)⁻ |
| 27 | H | n-Propyl (1R) | CN | 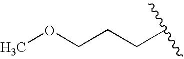 | CH₃ | CH₂ | bond | 399 (M − H)⁻ |
| 28 | H | n-Propyl (1R) | CN | 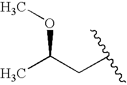 | CH₃ | CH₂ | bond | 399 (M − H)⁻ |
| 29 | H | n-Propyl (1R) | CN | 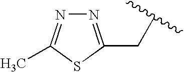 | CH₃ | CH₂ | bond | 441 (M + H)⁺ |
| 30 | H | n-Propyl (1R) | CN | 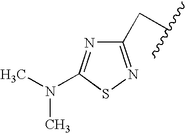 | CH₃ | CH₂ | bond | 470 (M + H)⁺ |
| 31 | H | n-Propyl | CN | 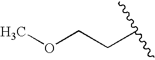 | CH₃ | bond | bond | 371 (M − H)⁻ |
| 32 | H | n-Propyl | CN | 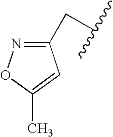 | CH₃ | bond | bond | 408 (M − H)⁻ |
| 33 | H | SecButyl | CN | 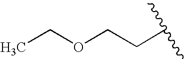 | CH₃ | CH₂ | bond | 413 (M − H)⁻ |
| 34 | H | SecButyl | CN | 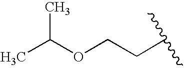 | CH₃ | CH₂ | bond | 429 (M + H)⁺ |
| 35 | H | SecButyl | CN | 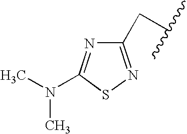 | CH₃ | CH₂ | bond | 484 (M − H)⁻ |
| 36 | H | SecButyl | CN | 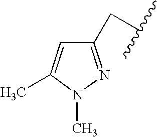 | CH₃ | CH₂ | bond | 449 (M − H)⁻ |
| 37 | H | n-Propyl | CN | 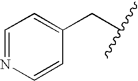 | CH₃ | bond | bond | 404 (M − H)⁻ |
| 38 | H | n-Propyl | CN | 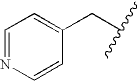 | CH₃ | CH₂ | bond | 420 (M + H)⁺ |
| 39 | H | n-Propyl | CN | 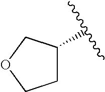 | CH₃ | CH₂ | bond | 399 (M + H)⁺ |
| 40 | H | n-Propyl | CN | 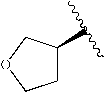 | CH₃ | CH₂ | bond | — |
| 41 | H | n-Propyl | CN | 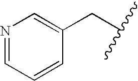 | CH₃ | CH₂ | bond | 420 (MH)⁺ |
| 42 | H | n-Propyl | CN | 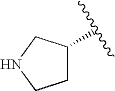 | CH₃ | CH₂ | bond | 398 (M + H)⁺ |
| 43 | H | n-Propyl | CN | 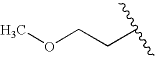 | CH₃ | CH₂ | bond | 387 (MH)⁺ |
| 44 | H | n-Propyl (1R) | CN | 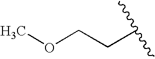 | CH₃ | CH₂ | bond | — |
| 45 | H | n-Propyl | CN | H | CH₃ | CH₂ | bond | 329 (M + H)⁺ |
| 46 | H | n-Propyl | CN | H | CH₃ | CH₂ | bond | — |
| | | (1R) | | | | | | |
| 47 | H | n-Propyl | CN | H | CH₃ | CH₂ | bond | — |
| | | (1S) | | | | | | |
| 48 | H | n-Propyl | CN | 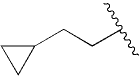 | CH₃ | CH₂ | bond | 395 (M − H)⁻ |
| 49 | H | n-Propyl | CN | 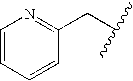 | CHs₃ | CH₂ | bond | 420 (M + H)⁺ |
| 50 | H | n-Propyl | CN | 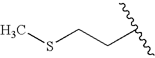 | CH₃ | CH₂ | bond | 401 (M − H)⁻ |
| 51 | H | n-Propyl | CN | 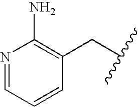 | CH₃ | CH₂ | bond | 435 (M + H)⁺ |
| 52 | H | n-Propyl | CN | 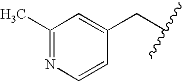 | CH₃ | CH₂ | bond | 434 (M + H)⁺ |
| 53 | H | n-Propyl (1R) | CN | 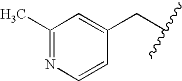 | CH₃ | CH₂ | bond | — |
| 54 | H | n-Propyl | CN | 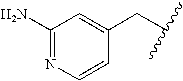 | CH₃ | CH₂ | bond | 435 (M + H)⁺ |
| 55 | H | n-Propyl | CN | 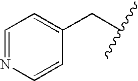 | CH₃ | CH₂ | bond | 434 (M + H)⁺ |
| 56 | H | n-Propyl | CN | 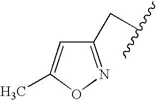 | CH₃ | CH₂ | bond | 422 (M − H)⁻ |
| 57 | H | n-Propyl (1S) | CN | 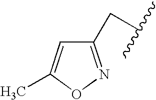 | CH₃ | CH₂ | bond | 424 (M + H)⁺ |
| 58 | H | n-Propyl | CN | 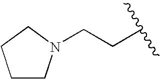 | CH₃ | CH₂ | bond | 424 (M − H)⁻ |
| 59 | H | n-Propyl | CN | 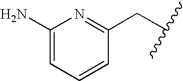 | CH₃ | CH₂ | bond | 435 (M + H)⁺ |
| 60 | H | n-Propyl | CN | 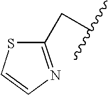 | CH₃ | CH₂ | bond | 426 (M + H)⁺ |
| 61 | H | n-Propyl | CN | 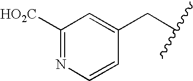 | CH₃ | CH₂ | bond | 464 (M + H)⁺ |
| 62 | H | n-Propyl | CN | 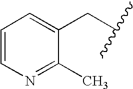 | CH₃ | CH₂ | bond | 432 (M + H)⁺ |
| 63 | H | n-Propyl | CN | 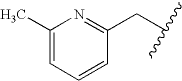 | CH₃ | CH₂ | bond | 432 (M + H)⁺ |
| 64 | H | n-Propyl | CN | 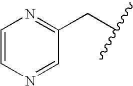 | CH₃ | CH₂ | bond | 421 (M + H)⁺ |
| 65 | H | n-Propyl | CN | 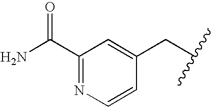 | CH₃ | CH₂ | bond | 463 (M + H)⁺ |
| 66 | H | n-Propyl | CN | 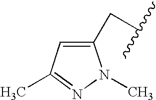 | CH₃ | CH₂ | bond | 437 (M + H)⁺ |
| 67 | H | n-Propyl | CN | 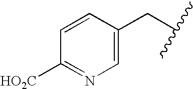 | CH₃ | CH₂ | bond | 448 (M + H)⁺ |
| 68 | H | n-Propyl | CN | 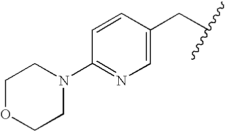 | CH₃ | CH₂ | bond | 503 (M − H)⁻ |
| 69 | H | n-Propyl | CN | 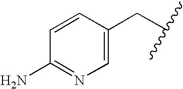 | CH₃ | CH₂ | bond | 433 (M − H)⁻ |
| 70 | H | n-Propyl | CN | 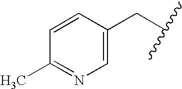 | CH₃ | CH₂ | bond | 434 (M + H)⁺ |
| 71 | H | n-Propyl | CN | 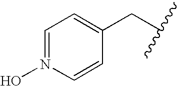 | CH₃ | CH₂ | bond | 436 (M + H)⁺ |
| 72 | H | n-Propyl | CN | 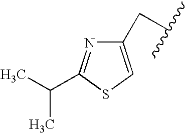 | CH₃ | CH₂ | bond | 466 (M − H)⁻ |
| 73 | H | n-Propyl | CN | 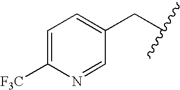 | CH₃ | CH₂ | bond | 488 (M + H)⁻ |
| 74 | H | n-Propyl | CN | 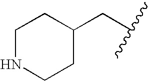 | CH₃ | CH₂ | bond | 424 (M − H)⁻ |
| 75 | H | n-Propyl | CN | 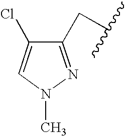 | CH₃ | CH₂ | bond | 457 (M + H)⁺ |
| 76 | H | n-Propyl | CN | 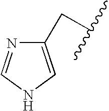 | CH₃ | CH₂ | bond | 409 (M + H)⁺ |
| 77 | H | n-Propyl | CN | 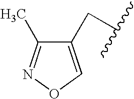 | CH₃ | CH₂ | bond | 424 (M + H)⁺ |
| 78 | H | n-Propyl | CN | 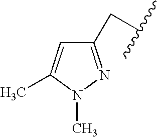 | CH₃ | CH₂ | bond | 437 (M + H)⁺ |
| 79 | H | n-Propyl | CN | 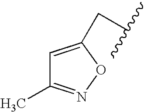 | CH₃ | CH₂ | bond | 422 (M − H)⁻ |
| 80 | H | n-Propyl | CN | 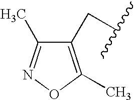 | CH₃ | CH₂ | bond | 436 (M − H)⁻ |
| 81 | H | n-Propyl | CN | 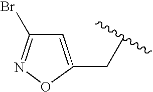 | CH₃ | CH₂ | bond | 488 (M − H)⁻ |
| 82 | H | n-Propyl | CN | 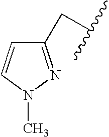 | CH₃ | CH₂ | bond | — |
| 83 | H | n-Propyl | CN | 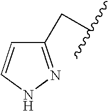 | CH₃ | CH₂ | bond | 407 (M − H)⁻ |
| 84 | H | n-Propyl | CN | 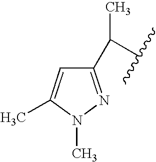 | CH₃ | CH₂ | bond | 451 (M + H)⁺ |
| 85 | H | n-Propyl | CN | 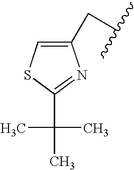 | CH₃ | CH₂ | bond | 482 (M + H)⁺ |
| 86 | H | n-Propyl | CN | 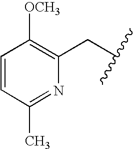 | CH₃ | CH₂ | bond | 464 (M + H)⁺ |
| 87 | H | n-Propyl | CN | 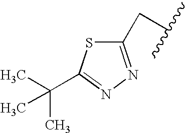 | CH₃ | CH₂ | bond | 483 (M + H)⁺ |
| 88 | H | n-Propyl | CN | 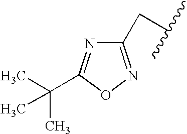 | CH₃ | CH₂ | bond | 467 (M + H)⁺ |
| 89 | H | n-Propyl | CN | 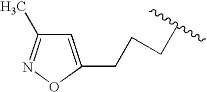 | CH₃ | CH₂ | bond | 450 (M − H)⁻ |
| 90 | H | n-Propyl | CN | 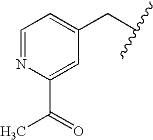 | CH₃ | CH₂ | bond | 460 (M − H)⁻ |
| 91 | H | n-Propyl | CN | 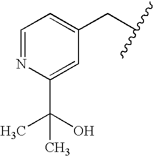 | CH₃ | CH₂ | bond | 476 (M − H)⁻ |
| 92 | H | n-Propyl | CN | 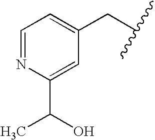 | CH₃ | CH₂ | bond | 462 (M − H)⁻ |
| 93 | H | n-Propyl | CN | 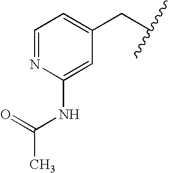 | CH₃ | CH₂ | bond | 475 (M − H)⁻ |
| 94 | H | n-Propyl | CN | 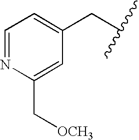 | CH₃ | CH₂ | bond | 462 (M − H)⁻ |
| 95 | H | n-Propyl | CN | 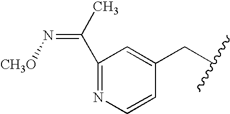 | CH₃ | CH₂ | bond | 489 (M − H)⁻ |
| 96 | H | n-Propyl | CN | 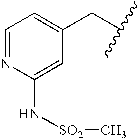 | CH₃ | CH₂ | bond | 511 (M − H)⁻ |
| 97 | H | n-Propyl | CN | 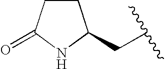 | CH₃ | CH₂ | bond | 424 (M − H)⁻ |
| 98 | H | n-Propyl | CN | 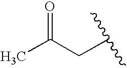 | CH₃ | CH₂ | bond | 383 (M − H)⁻ |
| 99 | H | n-Propyl | CN | 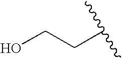 | CH₃ | CH₂ | bond | 371 (M − H)⁻ |
| 100 | H | n-Propyl | CN | 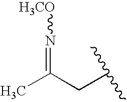 | CH₃ | CH₂ | bond | 412 (M − H)⁻ |
| 101 | H | n-Propyl | CN | 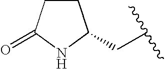 | CH₃ | CH₂ | bond | 424 (M − H)⁻ |
| 102 | H | n-Propyl | CN | 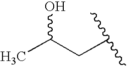 | CH₃ | CH₂ | bond | 385 (M − H)⁻ |
| 103 | H | n-Propyl | CN | 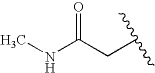 | CH₃ | CH₂ | bond | 398 (M − H)⁻ |
| 104 | H | n-Propyl | CN | 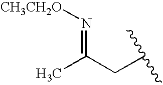 | CH₃ | CH₂ | bond | 426 (M − H)⁻ |
| 105 | H | n-Propyl | CN | 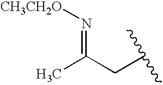 | CH₃ | CH₂ | bond | 426 (M − H)⁻ |
| 106 | H | n-Propyl | CN | 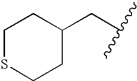 | CH₃ | CH₂ | bond | 441 (M − H)⁻ |
| 107 | H | n-Propyl | CN | 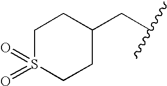 | CH₃ | CH₂ | bond | 473 (M − H)⁻ |
| 108 | H | n-Propyl | CN | 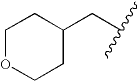 | CH₃ | CH₂ | bond | 425 (M − H)⁻ |
| 109 | H | n-Propyl | CN | 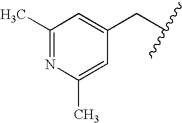 | CH₃ | CH₂ | bond | 446 (M + H)⁺ |
| 110 | H | n-Propyl | CN | 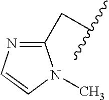 | CH₃ | CH₂ | bond | 421 (M − H)⁻ |
| 111 | H | n-Propyl | CN | 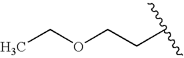 | CH₃ | CH₂ | bond | 401 (M + H)⁺ |
| 112 | H | n-Propyl | CN | 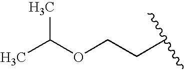 | CH₃ | CH₂ | bond | 415 (M + H)⁺ |
| 113 | H | n-Propyl | CN | 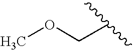 | CH₃ | CH₂ | bond | 373 (M + H)⁺ |
| 114 | H | n-Propyl | CN | 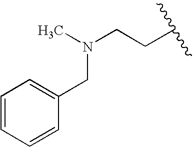 | CH₃ | CH₂ | bond | 476 (M + H)⁺ |
| 115 | H | n-Propyl | CN | 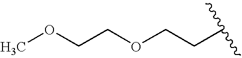 | CH₃ | CH₂ | bond | 431 (M + H)⁺ |
| 116 | H | n-Propyl | CN | 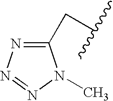 | CH₃ | CH₂ | bond | 423 (M − H)⁻ |
| 117 | H | n-Propyl | CN | 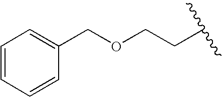 | CH₃ | CH₂ | bond | 463 (M + H)⁺ |
| 118 | H | n-Propyl (1R) | CN | 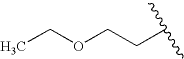 | CH₃ | CH₂ | bond | 399 (M − H)⁻ |
| 119 | H | n-Propyl | CN | 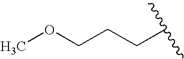 | CH₃ | CH₂ | bond | 399 (M − H)⁻ |
| 120 | H | n-Propyl | CN | 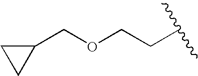 | CH₃ | CH₂ | bond | 425 (M − H)⁻ |
| 121 | H | n-Propyl | CN | 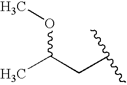 | CH₃ | CH₂ | bond | 399 (M − H)⁻ |
| 122 | H | n-Propyl | CN | 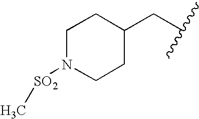 | CH₃ | CH₂ | bond | 502 (M − H)⁻ |
| 123 | H | n-Propyl | CN | 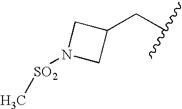 | CH₃ | CH₂ | bond | 474 (M − H)⁻ |
| 124 | H | n-Propyl | CN | 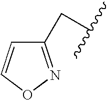 | CH₃ | CH₂ | bond | 408 (M − H)⁻ |
| 125 | H | n-Propyl (1R) | CN | 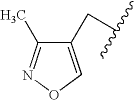 | CH₃ | CH₂ | bond | 422 (M − H)⁻ |
| 126 | H | n-Propyl | CN | 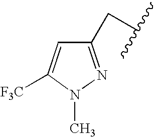 | CH₃ | CH₂ | bond | 489 (M − H)⁻ |
| 127 | H | n-Propyl | CN | 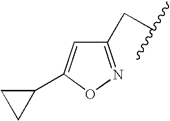 | CH₃ | CH₂ | bond | 448 (M − H)⁻ |
| 128 | H | n-Propyl | CN | 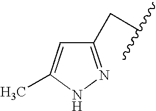 | CH₃ | CH₂ | bond | 421 (M − H)⁻ |
| 129 | H | n-Propyl | CN | 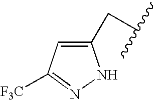 | CH₃ | CH₂ | bond | 475 (M − H)⁻ |
| 130 | H | n-Propyl | CN | 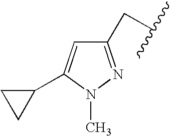 | CH₃ | CH₂ | bond | 461 (M − H)⁻ |
| 131 | H | n-Propyl | CN | 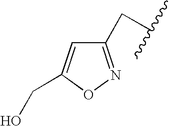 | CH₃ | CH₂ | bond | 438 (M − H)⁻ |
| 132 | H | n-Propyl | CN | 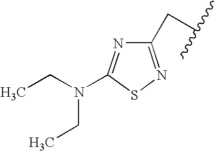 | CH₃ | CH₂ | bond | 496 (M − H)⁻ |
| 133 | H | n-Propyl | CN | 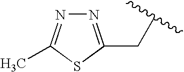 | CH₃ | CH₂ | bond | 441 (M + H)⁺ |
| 134 | H | n-Propyl | CN | 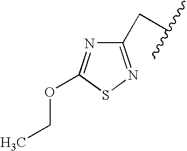 | CH₃ | CH₂ | bond | 469 (M + H)⁺ |
| 135 | H | n-Propyl | CN | 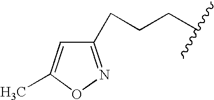 | CH₃ | CH₂ | bond | 452 (M + H)⁺ |
| 136 | H | n-Propyl | CN | 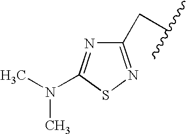 | CH₃ | CH₂ | bond | 470 (M + H)⁺ |
| 137 | H | n-Propyl | CN | 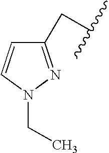 | CH₃ | CH₂ | bond | 437 (M + H)⁺ |
| 138 | H | n-Propyl | CN | 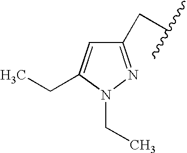 | CH₃ | CH₂ | bond | 463 (M − H)⁻ |
| 139 | H | n-Propyl | CN | 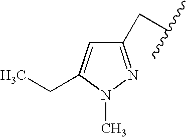 | CH₃ | CH₂ | bond | 449 (M − H)⁻ |
| 140 | H | n-Propyl | CN | 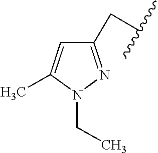 | CH₃ | CH₂ | bond | 449 (M − H)⁻ |
| 141 | H | n-Propyl | CN | 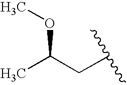 | CH₃ | CH₂ | bond | 401 (M + H)⁺ |
| 142 | H | n-Propyl | CN | 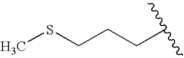 | CH₃ | CH₂ | bond | 415 (M − H)⁻ |
| 143 | H | n-Propyl | CN | 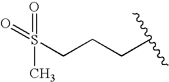 | CH₃ | CH₂ | bond | 447 (M − H)⁻ |

*Mass Spectroscopy data is expressed as a mass to charge ratio (m/z) for either (M + 1) or (M − 1) molecular ion.
**Compounds of Example No.s 45-47 are useful for making compounds of Formula I.

### EXAMPLE 1

(5-Cyano-7-hydroxymethyl-8-methyl-1-propyl-1,3,4,9-tetrahydro-pyrano[3,4-b]indol-1-yl)-acetic acid

### EXAMPLE 2

(5-Cyano-7-methoxymethyl-8-methyl-1-propyl-1,3,4,9-tetrahydro-pyrano[3,4-b]indol-1-yl)-acetic acid

### EXAMPLE 3

(5-Cyano-7-ethoxymethyl-8-methyl-1-propyl-1,3,4,9-tetrahydro-pyrano[3,4-b]indo-1-yl)-acetic acid

### EXAMPLE 4

(5-Cyano-8-methyl-7-propoxymethyl-1-propyl-1,3,4,9-tetrahydro-pyrano[3,4-b]indol-1-yl)-acetic acid

### EXAMPLE 5

(5-Cyano-7-isopropoxymethyl-8-methyl-1-propyl-1,3,4,9-tetrahydro-pyrano[3,4-b]indol-1-yl)-acetic acid

### EXAMPLE 6

(5-Cyano-7-cyclobutoxymethyl-8-methyl-1-propyl-1,3,4,9-tetrahydro-pyrano[3,4-b]indol-1-yl)-acetic acid

### EXAMPLE 7

(5-Cyano-7-cyclohexyloxymethyl-8-methyl-1-propyl-1,3,4,9-tetrahydro-pyrano[3,4-b]indol-1-yl)-acetic acid

### EXAMPLE 8

(5-Cyano-7-cyclopropylmethoxymethyl-8-methyl-1-propyl-1,3,4,9-tetrahydro-pyrano[3,4-b]indol-1-yl)-acetic acid

### EXAMPLE 9

(5-Cyano-7-cyclobutylmethoxymethyl-8-methyl-1-propyl-1,3,4,9-tetrahydro-pyrano[3,4-b]indol-1-yl)-acetic acid

### EXAMPLE 10

(5-Cyano-7-cyclopentylmethoxymethyl-8-methyl-1-propyl-1,3,4,9-tetrahydro-pyrano[3,4-b]indol-1-yl)-acetic acid

### EXAMPLE 11

(7-But-2-ynyloxymethyl-5-cyano-8-methyl-1-propyl-1,3,4,9-tetrahydro-pyrano[3,4-b]indol-1-yl)-acetic acid

### EXAMPLE 12

[5-Cyano-8-methyl-1-propyl-7-(tetrahydro-pyran-4-ylmethoxymethyl)-1,3,4,9-tetrahydro-pyrano[3,4-b]indol-1-yl]-acetic acid

### EXAMPLE 13

(3′S,1S*)[5-Cyano-8-methyl-1-propyl-7-(tetrahydro-furan-3-yloxymethyl)-1,3,4,9-tetrahydro-pyrano[3,4-b]indol-1-yl]-acetic acid

### EXAMPLE 14

(3′R,1S*)[5-Cyano-8-methyl-1-propyl-7-(tetrahydro-furan-3-yloxymethyl)-1,3,4,9-tetrahydro-pyrano[3,4-b]indol-1-yl]-acetic acid

### EXAMPLE 15

(7-Benzyloxymethyl-5-cyano-8-methyl-1-propyl-1,3,4,9-tetrahydro-pyrano[3,4-b]indol-1-yl)-acetic acid

### EXAMPLE 16

[7-(Benzo[1,3]dioxol-5-ylmethoxymethyl)-5-cyano-8-methyl-1-propyl-1,3,4,9-tetrahydro-pyrano[3,4-b]indol-1-yl]-acetic acid

### EXAMPLE 17

[5-Cyano-7-(2,4-dimethyl-benzyloxymethyl)-8-methyl-1-propyl-1,3,4,9-tetrahydro-pyrano[3,4-b]indol-1-yl]-acetic acid

### EXAMPLE 18

[5-Cyano-8-methyl-1-propyl-7-(thiophen-3-ylmethoxymethyl)-1,3,4,9-tetrahydro-pyrano[3,4-b]indol-1-yl]-acetic acid

### EXAMPLE 19

[5-Cyano-7-(2,4-dimethyl-thiazol-5-ylmethoxymethyl)-8-methyl-1-propyl-1,3,4,9-tetrahydro-pyrano[3,4-b]indol-1-yl]-acetic acid

### EXAMPLE 20

(5-Cyano-8-methyl-7-phenoxymethyl-1-propyl-1,3,4,9-tetrahydro-pyrano[3,4-b]indol-1-yl)-acetic acid

### EXAMPLE 21

[5-Cyano-7-(3-fluoro-phenoxymethyl)-8-methyl-1-propyl-1,3,4,9-tetrahydro-pyrano[3,4-b]indol-1-yl]-acetic acid

### EXAMPLE 22

(5-Cyano-7-cyclopropylmethoxy-8-methyl-1-propyl-1,3,4,9-tetrahydro-pyrano[3,4-b]indol-1-yl)-acetic acid

### EXAMPLE 23

(R)-[5-Cyano-8-methyl-7-(5-methyl-isoxazol-3-ylmethoxy)-1-propyl-1,3,4,9-tetrahydro-pyrano[3,4-b]indol-1-yl]-acetic acid

### EXAMPLE 24

[5-Cyano-8-methyl-1-propyl-7-(pyridin-4-ylmethoxy)-1,3,4,9-tetrahydro-pyrano[3,4-b]indol-1-yl]-acetic acid

### EXAMPLE 25

[5-Cyano-7-(1,5-dimethyl-1H-pyrazol-3-ylmethoxy)-8-methyl-1-propyl-1,3,4,9-tetrahydro-pyrano[3,4-b]indol-1-yl]-acetic acid

### EXAMPLE 26

(R)-[5-Cyano-7-(2-isopropoxy-ethoxy)-8-methyl-1-propyl-1,3,4,9-tetrahydro-pyrano[3,4-b]indol-1-yl]-acetic acid

### EXAMPLE 27

(R)-[5-Cyano-7-(3-methoxy-propoxy)-8-methyl-1-propyl-1,3,4,9-tetrahydro-pyrano[3,4-b]indol-1-yl]-acetic acid

### EXAMPLE 28

(1R,2′R)-[5-Cyano-7-(2-methoxy-propoxy)-8-methyl-1-propyl-1,3,4,9-tetrahydro-pyrano[3,4-b]indol-1-yl]-acetic acid

### EXAMPLE 29

[5-Cyano-8-methyl-7-(5-methyl-[1,3,4]thiadiazol-2-ylmethoxy)-1-propyl-1,3,4,9-tetrahydro-pyrano[3,4-b]indol 1-yl]-acetic acid

### EXAMPLE 30

(R)-[5-Cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid

### EXAMPLE 31

5-Cyano-7-(2-methoxy-ethoxy)-8-methyl-1-propyl-1,3,4,9-tetrahydro-pyrano[3,4,-b]indole-1-carboxylic acid

### EXAMPLE 32

5-Cyano-8-methyl-7-(5-methyl-isoxazol-3-ylmethoxy)-1-propyl-1,3,4,9-tetrahydro-pyrano[3,4,-b]indole-1-carboxylic acid

### EXAMPLE 33

(1R*,10S)-[1-sec-Butyl-5-cyano-7-(2-ethoxy-ethoxy)-8-methyl-1,3,4,9-tetrahydro-pyrano[3,4-b]indol-1-yl]-acetic acid

### EXAMPLE 34

(1R*,10S)-[1-sec-Butyl-5-cyano-7-(2-isopropoxy-ethoxy)-8-methyl-1,3,4,9-tetrahydro-pyrano[3,4-b]indol-1-yl]-acetic acid

### EXAMPLE 35

(1R*,10S)-[1-sec-Butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid

### EXAMPLE 36

(1R*,10S)-[1-sec-Butyl-5-cyano-7-(1,5-dimethyl-1H-pyrazol-3-ylmethoxy)-8-methyl-1,3,4,9-tetrahydro-pyrano[3,4-b]indol-1-yl]-acetic acid

What is claimed:

1. A compound having the formula: 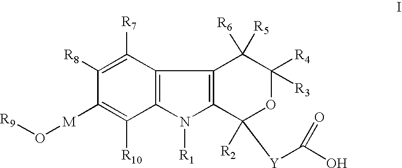
wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond, CH₂, or CH₂CH₂, with the proviso that when M is a bond, then R₉ is other than a hydroxyl, a straight chain alkyl of 1 to 8 carbon atoms, a branched alkyl of 3 to 12 carbons atoms, or an arylalkyl;
Y is a bond, CH₂, CH₂CH₂, aryl, or R₂ and Y together with the ring carbon atom to which they are attached may additionally form a spirocyclic cycloalkyl ring of 3 to 8 carbon atoms; or
a crystalline form or a pharmaceutically acceptable salt thereof.

2. The compound according to claim 1 wherein
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is CH₂ or CH₂CH₂;
Y is a bond, CH₂, CH₂CH₂, aryl, or R₂ and Y together with the ring carbon atom to which they are attached may additionally form a spirocyclic cycloalkyl ring of 3 to 8 carbon atoms; or
a crystalline form or a pharmaceutically acceptable salt thereof.

3. The compound according to claim 1 wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond;
Y is a bond, CH₂, CH₂CH₂, aryl, or R₂ and Y together with the ring carbon atom to which they are attached may additionally form a spirocyclic cycloalkyl ring of 3 to 8 carbon atoms; or
a crystalline form or a pharmaceutically acceptable salt thereof.

4. The compound according to claim 1 selected from the group consisting of:
(5-cyano-7-hydroxymethyl-8-methyl-1-propyl-1,3,4,9-tetrahydro-pyrano[3,4-b]indol-1-yl)-acetic acid;
(5-cyano-7-methoxymethyl-8-methyl-1-propyl-1,3,4,9-tetrahydro-pyrano[3,4-b]indol-1-yl)-acetic acid;
(5-cyano-7-ethoxymethyl-8-methyl-1-propyl-1,3,4,9-tetrahydro-pyrano[3,4-b]indol-1-yl)-acetic acid;
(5-cyano-8-methyl-7-propoxymethyl-1-propyl-1,3,4,9-tetrahydro-pyrano[3,4-b]indol-1-yl)-acetic acid;
(5-cyano-7-isopropoxymethyl-8-methyl-1-propyl-1,3,4,9-tetrahydro-pyrano[3,4-b]indol-1-yl)-acetic acid;
(5-cyano-7-cyclobutoxymethyl-8-methyl-1-propyl-1,3,4,9-tetrahydro-pyrano[3,4-b]indol-1-yl)-acetic acid;
(5-cyano-7-cyclohexyloxymethyl-8-methyl-1-propyl-1,3,4,9-tetrahydro-pyrano[3,4-b]indol-1-yl)-acetic acid;
(5-cyano-7-cyclopropylmethoxymethyl-8-methyl-1-propyl-1,3,4,9-tetrahydro-pyrano[3,4-b]indol-1-yl)-acetic acid;
(5-cyano-7-cyclobutylmethoxymethyl-8-methyl-1-propyl-1,3,4,9-tetrahydro-pyrano[3,4-b]indol-1-yl)-acetic acid;
(5-cyano-7-cyclopentylmethoxymethyl-8-methyl-1-propyl-1,3,4,9-tetrahydro-pyrano[3,4-b]indol-1-yl)-acetic acid;
(7-but-2-ynyloxymethyl-5-cyano-8-methyl-1-propyl-1,3,4,9-tetrahydro-pyrano[3,4-b]indol-1-yl)-acetic acid;
[5-cyano-8-methyl-1-propyl-7-(tetrahydro-pyran-4-ylmethoxymethyl)-1,3,4,9-tetrahydro-pyrano[3,4-b]indol-1-yl]-acetic acid;
(3′S,1S*)[5-cyano-8-methyl-1-propyl-7-(tetrahydro-furan-3-yloxymethyl)-1,3,4,9-tetrahydro-pyrano[3,4-b]indol-1-yl]-acetic acid;
(3′R,1S*)[5-cyano-8-methyl-1-propyl-7-(tetrahydro-furan-3-yloxymethyl)-1,3,4,9-tetrahydro-pyrano[3,4-b]indol-1-yl]-acetic acid;
(7-benzyloxymethyl-5-cyano-8-methyl-1-propyl-1,3,4,9-tetrahydro-pyrano[3,4-b]indol-1-yl)-acetic acid;
[7-(benzo[1,3]dioxol-5-ylmethoxymethyl)-5-cyano-1-methyl-1-propyl-1,3,4,9-tetrahydro-pyrano[3,4-b]indol-1-yl]-acetic acid;
[5-cyano-7-(2,4-dimethyl-benzyloxymethyl)-8-methyl-1-propyl-1,3,4,9-tetrahydro-pyrano[3,4-b]indol-1-yl]-acetic acid;
[5-cyano-8-methyl-1-propyl-7-(thiophen-3-ylmethoxymethyl)-1,3,4,9-tetrahydro-pyrano[3,4-b]indol-1-yl]-acetic acid;
[5-cyano-7-(2,4-dimethyl-thiazol-5-ylmethoxymethyl)-8-methyl-1-propyl-1,3,4,9-tetrahydro-pyrano[3,4-b]indol-1-yl]-acetic acid;
(5-cyano-8-methyl-7-phenoxymethyl-1-propyl-1,3,4,9-tetrahydro-pyrano[3,4-b]indol-1-yl)-acetic acid;
[5-cyano-7-(3-fluoro-phenoxymethyl)-8-methyl-1-propyl-1,3,4,9-tetrahydro-pyrano[3,4-b]indol-1-yl]-acetic acid;
(5-cyano-7-cyclopropylmethoxy-8-methyl-1-propyl-1,3,4,9-tetrahydro-pyrano[3,4-b]indol-1-yl)-acetic acid;
(R)-[5-cyano-8-methyl-7-(5-methyl-isoxazol-3-ylmethoxy)-1-propyl-1,3,4,9-tetrahydro-pyrano[3,4-b]indol-1-yl]-acetic acid;
[5-cyano-8-methyl-1-propyl-7-(pyridin-4-ylmethoxy)-1,3,4,9-tetrahydro-pyrano[3,4-b]indol-1-yl]-acetic acid;
[5-cyano-7-(1,5-dimethyl-1H-pyrazol-3-ylmethoxy)-8-methyl-1-propyl-1,3,4,9-tetrahydro-pyrano[3,4-b]indol-1-yl]-acetic acid;
(R)-[5-cyano-7-(2-isopropoxy-ethoxy)-8-methyl-1-propyl-1,3,4,9-tetrahydro-pyrano[3,4-b]indol-1-yl]-acetic acid;
(R)-[5-cyano-7-(3-methoxy-propoxy)-8-methyl-1-propyl-1,3,4,9-tetrahydro-pyrano[3,4-b]indol-1-yl]-acetic acid;
(1R,2′R)-[5-cyano-7-(2-methoxy-propoxy)-8-methyl-1-propyl-1,3,4,9-tetrahydro-pyrano[3,4-b]indol-1-yl]-acetic acid;
[5-cyano-8-methyl-7-(5-methyl-[1,3,4]thiadiazol-2-ylmethoxy)-1-propyl-1,3,4,9-tetrahydro-pyrano[3,4-b]indol-1-yl]-acetic acid;
(R)-[5-cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid;
5-cyano-7-(2-methoxy-ethoxy)-8-methyl-1-propyl-1,3,4,9-tetrahydro-pyrano[3,4,-b]indole-1-carboxylic acid;
5-cyano-8-methyl-7-(5-methyl-isoxazol-3-ylmethoxy)-1-propyl-1,3,4,9-tetrahydro-pyrano[3,4,-b]indole-1-carboxylic acid;
(1R*,10S)-[1-sec-butyl-5-cyano-7-(2-ethoxy-ethoxy)-8-methyl-1,3,4,9-tetrahydro-pyrano[3,4-b]indol-1-yl]-acetic acid;
(1R*,10S)-[1-sec-butyl-5-cyano-7-(2-isopropoxy-ethoxy)-8-methyl-1,3,4,9-tetrahydro-pyrano[3,4-b]indol-1-yl]-acetic acid;
(1R*,10S)-[1-sec-butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid; and
(1R*,10S)-[1-sec-butyl-5-cyano-7-(1,5-dimethyl-1H-pyrazol-3-ylmethoxy)-8-methyl-1,3,4,9-tetrahydro-pyrano[3,4-b]indol-1-yl]-acetic acid.

5. The compound according to claim 1 having the formula:

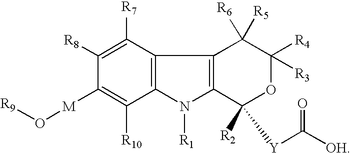

6. A pharmaceutical composition comprising a compound of a formula: 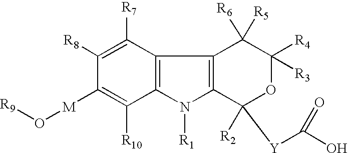
wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇—R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond, CH₂, or CH₂CH₂, with the proviso that when M is a bond, then R₉ is other than a hydroxyl, a straight chain alkyl of 1 to 8 carbon atoms, a branched alkyl of 3 to 12 carbons atoms, or an arylalkyl;
Y is a bond, CH₂, CH₂CH₂, aryl, or R₂ and Y together with the ring carbon atom to which they are attached may additionally form a spirocyclic cycloalkyl ring of 3 to 8 carbon atoms;
or a crystalline form or a pharmaceutically acceptable salt thereof; and a pharmaceutically acceptable carrier.

7. A method of treating or preventing a Hepatitis C viral infection in a mammal comprising providing the mammal with an effective amount of a compound of a formula: 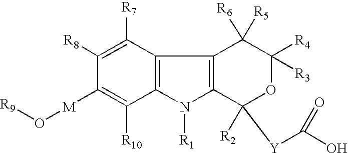
wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond, CH₂, or CH₂CH₂, with the proviso that when M is a bond, then R₉ is other than a hydroxyl, a straight chain alkyl of 1 to 8 carbon atoms, a branched alkyl of 3 to 12 carbons atoms, or an arylalkyl;
Y is a bond, CH₂, CH₂CH₂, aryl, or R₂ and Y together with the ring carbon atom to which they are attached may additionally form a spirocyclic cycloalkyl ring of 3 to 8 carbon atoms; or
a crystalline form or a pharmaceutically acceptable salt thereof.

8. The method of claim 7 wherein the compound is selected from the group consisting of
(5-cyano-7-hydroxymethyl-8-methyl-1-propyl-1,3,4,9-tetrahydro-pyrano[3,4-b]indol-1-yl)-acetic acid;
(5-cyano-7-methoxymethyl-8-methyl-1-propyl-1,3,4,9-tetrahydro-pyrano[3,4-b]indol-1-yl)-acetic acid;
(5-cyano-7-ethoxymethyl-8-methyl-1-propyl-1,3,4,9-tetrahydro-pyrano[3,4-b]indol-1-yl)-acetic acid;
(5-cyano-8-methyl-7-propoxymethyl-1-propyl-1,3,4,9-tetrahydro-pyrano[3,4-b]indol-1-yl)-acetic acid;
(5-cyano-7-isopropoxymethyl-8-methyl-1-propyl-1,3,4,9-tetrahydro-pyrano[3,4-b]indol-1-yl)-acetic acid;
(5-cyano-7-cyclobutoxymethyl-8-methyl-1-propyl-1,3,4,9-tetrahydro-pyrano[3,4-b]indol-1-yl)-acetic acid;
(5-cyano-7-cyclohexyloxymethyl-8-methyl-1-propyl-1,3,4,9-tetrahydro-pyrano[3,4-b]indol-1-yl)-acetic acid;
(5-cyano-7-cyclopropylmethoxymethyl-8-methyl-1-propyl-1,3,4,9-tetrahydro-pyrano[3,4-b]indol-1-yl)-acetic acid;
(5-cyano-7-cyclobutylmethoxymethyl-8-methyl-1-propyl-1,3,4,9-tetrahydro-pyrano[3,4-b]indol-1-yl)-acetic acid;
(5-cyano-7-cyclopentylmethoxymethyl-8-methyl-1-propyl-1,3,4,9-tetrahydro-pyrano[3,4-b]indol-1-yl)-acetic acid;
(7-but-2-ynyloxymethyl-5-cyano-8-methyl-1-propyl-1,3,4,9-tetrahydro-pyrano[3,4-b]indol-1-yl)-acetic acid;
[5-cyano-8-methyl-1-propyl-7-(tetrahydro-pyran-4-ylmethoxymethyl)-1,3,4,9-tetrahydro-pyrano[3,4-b]indol-1-yl]-acetic acid;
(3′S,1S*)[5-cyano-8-methyl-1-propyl-7-(tetrahydro-furan-3-yloxymethyl)-1,3,4,9-tetrahydro-pyrano[3,4-b]indol-1-yl]-acetic acid;
(3′R,1S*)[5-cyano-8-methyl-1-propyl-7-(tetrahydro-furan-3-yloxymethyl)-1,3,4,9-tetrahydro-pyrano[3,4-b]indol-1-yl]-acetic acid;
(7-benzyloxymethyl-5-cyano-8-methyl-1-propyl-1,3,4,9-tetrahydro-pyrano[3,4-b]indol 1-yl)-acetic acid;
[7-(benzo[1,3]dioxol-5-ylmethoxymethyl)-5-cyano-8-methyl-1-propyl-1,3,4,9-tetrahydro-pyrano[3,4-b]indol-1-yl]-acetic acid;
[5-cyano-7-(2,4-dimethyl-benzyloxymethyl)-8-methyl-1-propyl-1,3,4,9-tetrahydro-pyrano[3,4-b]indol-1-yl]-acetic acid;
[5-cyano-8-methyl-1-propyl-7-(thiophen-3-ylmethoxymethyl)-1,3,4,9-tetrahydro-pyrano[3,4-b]indol-1-yl]-acetic acid;
[5-cyano-7-(2,4-dimethyl-thiazol-5-ylmethoxymethyl)-8-methyl-1-propyl-1,3,4,9-tetrahydro-pyrano[3,4-b]indol-1-yl]-acetic acid;
(5-cyano-8-methyl-7-phenoxymethyl-1-propyl-1,3,4,9-tetrahydro-pyrano[3,4-b]indol-1-yl)-acetic acid;
[5-cyano-7-(3-fluoro-phenoxymethyl)-8-methyl-1-propyl-1,3,4,9-tetrahydro-pyrano[3,4-b]indol-1-yl]-acetic acid;
(5-cyano-7-cyclopropylmethoxy-8-methyl-1-propyl-1,3,4,9-tetrahydro-pyrano[3,4-b]indol-1-yl)-acetic acid;
(R)-[5-cyano-8-methyl-7-(5-methyl-isoxazol-3-ylmethoxy)-1-propyl-1,3,4,9-tetrahydro-pyrano[3,4-b]indol-1-yl]-acetic acid;
[5-cyano-8-methyl-1-propyl-7-(pyridin-4-ylmethoxy)-1,3,4,9-tetrahydro-pyrano[3,4-b]indol-1-yl]-acetic acid;
[5-cyano-7-(1,5-dimethyl-1H-pyrazol-3-ylmethoxy)-8-methyl-1-propyl-1,3,4,9-tetrahydro-pyrano[3,4-b]indol-1-yl]-acetic acid;
(R)-[5-cyano-7-(2-isopropoxy-ethoxy)-8-methyl-1-propyl-1,3,4,9-tetrahydro-pyrano[3,4-b]indol-1-yl]-acetic acid;
(R)-[5-cyano-7-(3-methoxy-propoxy)-8-methyl-1-propyl-1,3,4,9-tetrahydro-pyrano[3,4-b]indol-1-yl]-acetic acid;
(1R,2′R)-[5-cyano-7-(2-methoxy-propoxy)-8-methyl-1-propyl-1,3,4,9-tetrahydro-pyrano[3,4-b]indol-1-yl]-acetic acid;
[5-cyano-8-methyl-7-(5-methyl-[1,3,4]thiadiazol-2-ylmethoxy)-1-propyl-1,3,4,9-tetrahydro-pyrano[3,4-b]indol-1-yl]-acetic acid;
(R)-[5-cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid;
5-cyano-7-(2-methoxy-ethoxy)-8-methyl-1-propyl-1,3,4,9-tetrahydro-pyrano[3,4,-b]indole-1-carboxylic acid;
5-cyano-8-methyl-7-(5-methyl-isoxazol-3-ylmethoxy)-1-propyl-1,3,4,9-tetrahydro-pyrano[3,4,-b]indole-1-carboxylic acid;
(1R*,10S)-[1-sec-butyl-5-cyano-7-(2-ethoxy-ethoxy)-8-methyl-1,3,4,9-tetrahydro-pyrano[3,4-b]indol-1-yl]-acetic acid;
(1R*,10S)-[1-sec-butyl-5-cyano-7-(2-isopropoxy-ethoxy)-8-methyl-1,3,4,9-tetrahydro-pyrano[3,4-b]indol-1-yl]-acetic acid;
(1R*,10S)-[1-sec-butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid; and
(1R*,10S)-[1-sec-butyl-5-cyano-7-(1,5-dimethyl-1H-pyrazol-3-ylmethoxy)-8-methyl-1,3,4,9-tetrahydro-pyrano[3,4-b]indol-1-yl]-acetic acid.

9. The method of claim 7 wherein the compound of the formula has a ratio of Isomer A to Isomer B of greater than 1:1, wherein Isomer A and Isomer B have the respective formulas:

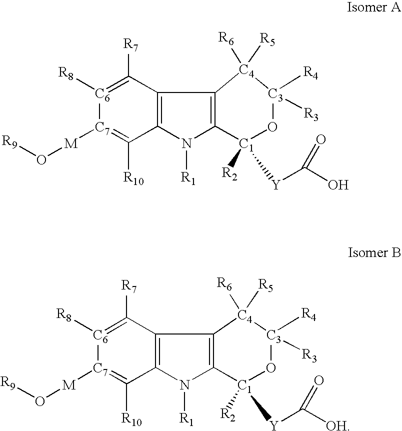

10. The method of claim 7 herein the compound of the formula is 100% Isomer A.

11. The method of claim 7 wherein the compound of, the formula has a ratio of Isomer A to Isomer B of at least 9:1.

12. The method of claim 7 wherein the compound of the formula has a ratio of Isomer A to Isomer B of at least 8:1.

13. The method of claim 7 wherein the compound of the formula has a ratio of Isomer A to Isomer B of at least 7:1.

14. A method of inhibiting replication of a Hepatitis C virus comprising contacting the Hepatitis C virus with a compound of a formula: 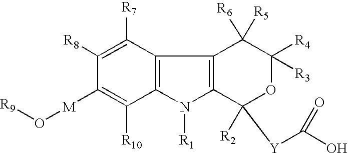
wherein:
R₁ is hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is hydrogen, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently hydrogen, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₈ and R₁₀ are independently hydrogen, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 6 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₉ is hydrogen, a straight chain alkyl of 1 to 8 carbon atoms, a branched alkyl of 3 to 12 carbons atoms, a cycloalkyl of 3 to 12 carbon atoms, a cycloalkyl-alkyl of 4 to 24 carbon atoms, an alkenyl of 2 to 7 carbon atoms, an alkynyl of 2 to 7 carbon atoms, an alkoxyalkyl of 2 to 12 carbon atoms, an alkoxyalkoxyalkyl of 3 to 18 carbon atoms, an arylalkoxyalkyl of 3 to 18 carbon atoms, a cycloalkylalkoxyalkyl of 3 to 18 carbon atoms, an aryloxyalkyl of 3 to 18 carbon atoms, a heteroaryloxyalkyl of 3 to 18 carbon atoms, an arylthioalkyl of 3 to 18 carbon atoms, a heteroarylthioalkyl of 3 to 18 carbon atoms, a hydroxyalkyl of 1 to 12 carbon atoms, an alkoxyiminoalkyl of 2 to 16 carbon atoms, an alkylthioalkyl of 2 to 16 carbon atoms, an alkylsulfonylalkyl group of 2 to 16 carbon atoms, a monoalkylaminoalkyl of 2 to 16 carbon atoms, a dialkylaminoalkyl of 3 to 16 carbon atoms, a substituted dialkylaminoalkyl of 3 to 16 carbon atoms, a substituted or unsubstituted aryl, arylalkyl of 7 to 12 carbon atoms, a substituted or unsubstituted heteroaryl of 7 to 12 carbon atoms, a substituted or unsubstituted heteroarylalkyl, a substituted or unsubstituted heterocyclic group, and a heterocycle-alkyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
M is a bond, CH₂, or CH₂CH₂, with the proviso that when M is a bond, then R₉ is other than a hydroxyl, a straight chain alkyl of 1 to 8 carbon atoms, a branched alkyl of 3 to 12 carbons atoms, or an arylalkyl;
Y is a bond, CH₂, CH₂CH₂, aryl, or R₂ and Y together with the ring carbon atom to which they are attached may additionally form a spirocyclic cycloalkyl ring of 3 to 8 carbon atoms; or
a crystalline form or a pharmaceutically acceptable salt thereof.

15. The method of claim 14 wherein the compound of the formula has a ratio of Isomer A to Isomer B of greater than 1:1, wherein Isomer A and Isomer B have the respective formulas:

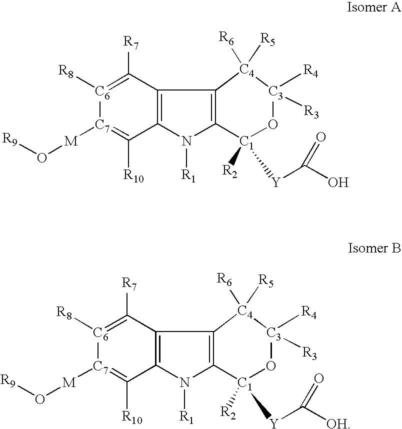

16. The method of claim 14 wherein the compound of the formula has a ratio of R-enantiomer to S-enantiomer of greater than 1:1.

17. The method of claim 14 wherein the compound of the formula is 100% Isomer A.

18. The method of claim 14 wherein the compound of the formula has a ratio of Isomer A to Isomer B of at least 9:1.

19. The method of claim 14 wherein the compound of the formula has a ratio of Isomer A to Isomer B of at least 8:1.

20. The method of claim 14 wherein the compound of the formula has a ratio of Isomer A to Isomer B of at least 7:1.

21. The method of claim 14 wherein the compound is selected from:
(5-cyano-7-hydroxymethyl-8-methyl-1-propyl-1,3,4,9-tetrahydro-pyrano[3,4-b]indol-1-yl)-acetic acid;
(5-cyano-7-methoxymethyl-8-methyl-1-propyl-1,3,4,9-tetrahydro-pyrano[3,4-b]indol-1-yl)-acetic acid;
(5-cyano-7-ethoxymethyl-8-methyl-1-propyl-1,3,4,9-tetrahydro-pyrano[3,4-b]indol-1-yl)-acetic acid;
(5-cyano-8-methyl-7-propoxymethyl-1-propyl-1,3,4,9-tetrahydro-pyrano[3,4-b]indol-1-yl)-acetic acid;
(5-cyano-7-isopropoxymethyl-8-methyl-1-propyl-1,3,4,9-tetrahydro-pyrano[3,4-b]indol-1-yl)-acetic acid;
(5-cyano-7-cyclobutoxymethyl-8-methyl-1-propyl-1,3,4,9-tetrahydro-pyrano[3,4-b]indol-1-yl)-acetic acid;
(5-cyano-7-cyclohexyloxymethyl-8-methyl-1-propyl-1,3,4,9-tetrahydro-pyrano[3,4-b]indol-1-yl)-acetic acid;
(5-cyano-7-cyclopropylmethoxymethyl-8-methyl-1-propyl-1,3,4,9-tetrahydro-pyrano[3,4-b]indol-1-yl)-acetic acid;
(5-cyano-7-cyclobutylmethoxymethyl-8-methyl-1-propyl-1,3,4,9-tetrahydro-pyrano[3,4-b]indol-1-yl)-acetic acid;
(5-cyano-7-cyclopentylmethoxymethyl-8-methyl-1-propyl-1,3,4,9-tetrahydro-pyrano[3,4-b]indol-1-yl)-acetic acid;
(7-but-2-ynyloxymethyl-5-cyano-8-methyl-1-propyl-1,3,4,9-tetrahydro-pyrano[3,4-b]indol-1-yl)-acetic acid;
[5-cyano-8-methyl-1-propyl-7-(tetrahydro-pyran-4-ylmethoxymethyl)-1,3,4,9-tetrahydro-pyrano[3,4-b]indol-1-yl]-acetic acid;
(3′S,1S*)[5-cyano-8-methyl-1-propyl-7-(tetrahydro-furan-3-yloxymethyl)-1,3,4,9-tetrahydro-pyrano[3,4-b]indol-1-yl]-acetic acid;
(3′R,1S*)[5-cyano-8-methyl-1-propyl-7-(tetrahydro-furan-3-yloxymethyl)-1,3,4,9-tetrahydro-pyrano[3,4-b]indol-1-yl]-acetic acid;
(7-benzyloxymethyl-5-cyano-8-methyl-1-propyl-1,3,4,9-tetrahydro-pyrano[3,4-b]indol-1-yl)-acetic acid;
[7-(benzo[1,3]dioxol-5-ylmethoxymethyl)-5-cyano-8-methyl-1-propyl-1,3,4,9-tetrahydro-pyrano[3,4-b]indol-1-yl]-acetic acid;
[5-cyano-7-(2,4-dimethyl-benzyloxymethyl)-8-methyl-1-propyl-1,3,4,9-tetrahydro-pyrano[3,4-b]indol-1-yl]-acetic acid;
[5-cyano-8-methyl-1-propyl-7-(thiophen-3-ylmethoxymethyl)-1,3,4,9-tetrahydro-pyrano[3,4-b]indol-1-yl]-acetic acid;
[5-cyano-7-(2,4-dimethyl-thiazol-5-ylmethoxymethyl)-8-methyl-1-propyl-1,3,4,9-tetrahydro-pyrano[3,4-b]indol-1-yl]-acetic acid;
(5-cyano-8-methyl-7-phenoxymethyl-1-propyl-1,3,4,9-tetrahydro-pyrano[3,4-b]indol-1-yl)-acetic acid;
[5-cyano-7-(3-fluoro-phenoxymethyl)-8-methyl-1-propyl-1,3,4,9-tetrahydro-pyrano[3,4-b]indol-1-yl]-acetic acid;
(5-cyano-7-cyclopropylmethoxy-8-methyl-1-propyl-1,3,4,9-tetrahydro-pyrano[3,4-b]indol-1-yl)-acetic acid;
(R)-[5-cyano-8-methyl-7-(5-methyl-isoxazol-3-ylmethoxy)-1-propyl-1,3,4,9-tetrahydro-pyrano[3,4-b]indol-1-yl]-acetic acid;
[5-cyano-8-methyl-1-propyl-7-(pyridin-4-ylmethoxy)-1,3,4,9-tetrahydro-pyrano[3,4-b]indol-1-yl]-acetic acid;
[5-cyano-7-(1,5-dimethyl-1H-pyrazol-3-ylmethoxy)-8-methyl-1-propyl-1,3,4,9-tetrahydro-pyrano[3,4-b]indol-1-yl]-acetic acid;
(R)-[5-cyano-7-(2-isopropoxy-ethoxy)-8-methyl-1-propyl-1,3,4,9-tetrahydro-pyrano[3,4-b]indol-1-yl]-acetic acid;
(R)-[5-cyano-7-(3-methoxy-propoxy)-8-methyl-1-propyl-1,3,4,9-tetrahydro-pyrano[3,4-b]indol-1-yl]-acetic acid;
(1R,2′R)-[5-cyano-7-(2-methoxy-propoxy)-8-methyl-1-propyl-1,3,4,9-tetrahydro-pyrano[3,4-b]indol-1-yl]-acetic acid;
[5-cyano-8-methyl-7-(5-methyl-[1,3,4]thiadiazol-2-ylmethoxy)-1-propyl-1,3,4,9-tetrahydro-pyrano[3,4-b]indol-1-yl]-acetic acid;
(R)-[5-cyano-7-(5-dimethylamino-[1,2,4]thiadiazol-3-ylmethoxy)-8-methyl-1-propyl-1,3,4,9-tetrahydro-pyrano[3,4-b]indol-1-yl]-acetic acid;
5-cyano-7-(2-methoxy-ethoxy)-8-methyl-1-propyl-1,3,4,9-tetrahydro-pyrano[3,4,-b]indole-1-carboxylic acid;
5-cyano-8-methyl-7-(5-methyl-isoxazol-3-ylmethoxy)-1-propyl-1,3,4,9-tetrahydro-pyrano[3,4,-b]indole-1-carboxylic acid;
(1R*,10S)-[1-sec-butyl-5-cyano-7-(2-ethoxy-ethoxy)-8-methyl-1,3,4,9-tetrahydro-pyrano[3,4-b]indol-1-yl]-acetic acid;
(1R*,10S)-[1-sec-butyl-5-cyano-7-(2-isopropoxy-ethoxy)-8-methyl-1,3,4,9-tetrahydro-pyrano[3,4-b]indol-1-yl]-acetic acid;
(1R*,10S)-[1-sec-butyl-5-cyano-7-(5-dimethylamino-[1,2,4]thiadiazol-3-ylmethoxy)-8-methyl-1,3,4,9-tetrahydro-pyrano[3,4-b]indol-1-yl]-acetic acid; and
(1R*,10S)-[1-sec-butyl-5-cyano-7-(1,5-dimethyl-1H-pyrazol-3-ylmethoxy)-8-methyl-1,3,4,9-tetrahydro-pyrano[3,4-b]indol-1-yl]-acetic acid.
